(19) United States
(12) Patent Application Publication
(10) Pub. No.: US 2026/0337282 A1
(43) Pub. Date: Oct. 29, 2026

(54) ANTI-CCR8 ANTIBODIES AND USES THEREOF

(71) Applicant: Sichuan Kelun-Biotech Biopharmaceutical Co., Ltd., Sichuan (CN)

(72) Inventors: Meimei Yin, Beijing (CN); Huichao Liang, Beijing (CN); Yanan Guo, Beijing (CN); Aki Kawagishi, Tokyo (JP); Takaho Terada, Tokyo (JP)

(21) Appl. No.: 19/123,094

(22) PCT Filed: Oct. 26, 2023
(86) PCT No.: PCT/CN2023/126829

§ 371 Date: Apr. 22, 2025

(30) Foreign Application Priority Data
Oct. 28, 2022 (WO) PCT/CN2022/128366
Aug. 23, 2023 (WO) PCT/CN2023/114451

(51) Int. Cl. C07K 16/28; A61K 39/00; A61K 47/64; A61K 47/68; A61P 35/00

(52) U.S. Cl. CPC C07K 16/2866; A61K 47/6415; A61K 47/6849; A61P 35/00; C07K 16/2818; C07K 16/2827; A61K 2039/507; C07K 2317/14; C07K 2317/24; C07K 2317/565

(57) ABSTRACT

Provided are anti-CCR8 (chemokine (C-C motif) receptor 8) antibodies, antigen-binding fragments, and the uses thereof.

### CLAIM OF PRIORITY

This application claims priority to PCT/CN2022/128366, filed on Oct. 28, 2022, and PCT/CN2023/114451, filed on Aug. 23, 2023. The entire contents of the foregoing application are incorporated herein by reference.

### TECHNICAL FIELD

This disclosure relates to anti-CCR8 (chemokine (C-C motif) receptor 8) antibodies and uses thereof.

### BACKGROUND

Regulatory T (Treg) cells suppress abnormal/excessive immune responses to self-antigens and nonself-antigens to maintain immune homeostasis. In tumor immunity, Treg cells are involved in tumor development and progression by inhibiting antitumor immunity.

Recent clinical and commercial success of anticancer antibodies has created great interest in antibody-based therapeutics, including those that target Treg. There is a need to develop anti-cancer antibodies that target Treg for use in various antibody-based therapeutics to treat cancers.

### SUMMARY

This disclosure relates to anti-CCR8 antibodies, antigen-binding fragment thereof, and the uses thereof. In one aspect, the present disclosure provides full human anti-CCR8 antibodies or antigen binding fragments thereof (e.g., that are produced by genetically modified non-human animals with a humanized heavy chain immunoglobulin locus and a humanized light chain immunoglobulin locus).

In one aspect, the disclosure is related to an antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDRs 1, 2, 3 amino acid sequences are set forth in SEQ ID NOs: 1, 2, 3, respectively,
(2) the selected VH CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively;
(4) the selected VH CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively;
(6) the selected VH CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 72, 73, 74, respectively;
(8) the selected VH CDRs 1, 2, 3 amino acid sequences are set forth in SEQ ID NOs: 75, 76, 77, respectively;
(9) the selected VH CDRs 1, 2, 3 amino acid sequences are set forth in SEQ ID NOs: 78, 79, 80, respectively;
(10) the selected VH CDRs 1, 2, 3 amino acid sequences are set forth in SEQ ID NOs: 81, 82, 83, respectively;
(11) the selected VH CDRs 1, 2, 3 amino acid sequences are set forth in SEQ ID NOs: 84, 85, 86, respectively;
(12) the selected VH CDRs 1, 2, 3 amino acid sequences are set forth in SEQ ID NOs: 87, 88, 89, respectively;
(13) the selected VH CDRs 1, 2, 3 amino acid sequences are set forth in SEQ ID NOs: 90, 91, 92, respectively;
(14) the selected VH CDRs 1, 2, 3 amino acid sequences are set forth in SEQ ID NOs: 93, 94, 95, respectively;
(15) the selected VH CDRs 1, 2, 3 amino acid sequences are set forth in SEQ ID NOs: 96, 97, 98, respectively;
(16) the selected VH CDRs 1, 2, 3 amino acid sequences are set forth in SEQ ID NOs: 99, 100, 101, respectively;
(17) the selected VH CDRs 1, 2, 3 amino acid sequences are set forth in SEQ ID NOs: 102, 103, 104, respectively;
(18) the selected VH CDRs 1, 2, 3 amino acid sequences are set forth in SEQ ID NOs: 105, 106, 107, respectively;
(19) the selected VH CDRs 1, 2, 3 amino acid sequences are set forth in SEQ ID NOs: 144, 145, 146, respectively;
(20) the selected VH CDRs 1, 2, 3 amino acid sequences are set forth in SEQ ID NOs: 147, 148, 149, respectively;
(21) the selected VH CDRs 1, 2, 3 amino acid sequences are set forth in SEQ ID NOs: 150, 151, 152, respectively;
(22) the selected VH CDRs 1, 2, 3 amino acid sequences are set forth in SEQ ID NOs: 153, 154, 155, respectively;
(23) the selected VH CDRs 1, 2, 3 amino acid sequences are set forth in SEQ ID NOs: 156, 157, 158, respectively;
(24) the selected VH CDRs 1, 2, 3 amino acid sequences are set forth in SEQ ID NOs: 159, 160, 161, respectively;
(25) the selected VH CDRs 1, 2, 3 amino acid sequences are set forth in SEQ ID NOs: 162, 163, 164, respectively;
(26) the selected VH CDRs 1, 2, 3 amino acid sequences are set forth in SEQ ID NOs: 165, 166, 167, respectively;
(27) the selected VH CDRs 1, 2, 3 amino acid sequences are set forth in SEQ ID NOs: 168, 169, 170, respectively;
(28) the selected VH CDRs 1, 2, 3 amino acid sequences are set forth in SEQ ID NOs: 171, 172, 173, respectively;
(29) the selected VH CDRs 1, 2, 3 amino acid sequences are set forth in SEQ ID NOs: 174, 175, 176, respectively;
(30) the selected VH CDRs 1, 2, 3 amino acid sequences are set forth in SEQ ID NOs: 177, 178, 179, respectively;
(31) the selected VH CDRs 1, 2, 3 amino acid sequences are set forth in SEQ ID NOs: 219, 220, 221, respectively;
(32) the selected VH CDRs 1, 2, 3 amino acid sequences are set forth in SEQ ID NOs: 222, 223, 224, respectively;
(33) the selected VH CDRs 1, 2, 3 amino acid sequences are set forth in SEQ ID NOs: 225, 226, 227, respectively;
(34) the selected VH CDRs 1, 2, 3 amino acid sequences are set forth in SEQ ID NOs: 228, 229, 230, respectively;
(35) the selected VH CDRs 1, 2, 3 amino acid sequences are set forth in SEQ ID NOs: 231, 232, 233, respectively;
(36) the selected VH CDRs 1, 2, 3 amino acid sequences are set forth in SEQ ID NOs: 234, 235, 236, respectively;
(37) the selected VH CDRs 1, 2, 3 amino acid sequences are set forth in SEQ ID NOs: 237, 238, 239, respectively;
(38) the selected VH CDRs 1, 2, 3 amino acid sequences are set forth in SEQ ID NOs: 240, 241, 242, respectively;
(39) the selected VH CDRs 1, 2, 3 amino acid sequences are set forth in SEQ ID NOs: 243, 244, 245, respectively;
(40) the selected VH CDRs 1, 2, 3 amino acid sequences are set forth in SEQ ID NOs: 246, 247, 248, respectively;
(41) the selected VH CDRs 1, 2, 3 amino acid sequences are set forth in SEQ ID NOs: 249, 250, 251, respectively;
(42) the selected VH CDRs 1, 2, 3 amino acid sequences are set forth in SEQ ID NOs: 252, 253, 254, respectively;
(43) the selected VH CDRs 1, 2, 3 amino acid sequences are set forth in SEQ ID NOs: 255, 256, 257, respectively; and
(44) the selected VH CDRs 1, 2, 3 amino acid sequences are set forth in SEQ ID NOs: 258, 259, 260, respectively;
wherein the selected VL CDRs, 1, 2, and 3 amino acid sequences are one of the following:
(1) the selected VL CDRs 1, 2, 3 amino acid sequences are set forth in SEQ ID NOs: 19, 20, 21, respectively;
(2) the selected VL CDRs 1, 2, 3 amino acid sequences are set forth in SEQ ID NOs: 22, 23, 24, respectively;
(3) the selected VL CDRs 1, 2, 3 amino acid sequences are set forth in SEQ ID NOs: 25, 26, 27, respectively;
(4) the selected VL CDRs 1, 2, 3 amino acid sequences are set forth in SEQ ID NOs: 28, 29, 30, respectively;
(5) the selected VL CDRs 1, 2, 3 amino acid sequences are set forth in SEQ ID NOs: 31, 32, 33, respectively;
(6) the selected VL CDRs 1, 2, 3 amino acid sequences are set forth in SEQ ID NOs: 34, 35, 36, respectively;
(7) the selected VL CDRs 1, 2, 3 amino acid sequences are set forth in SEQ ID NOs: 108, 109, 110, respectively;
(8) the selected VL CDRs 1, 2, 3 amino acid sequences are set forth in SEQ ID NOs: 111, 112, 113, respectively;
(9) the selected VL CDRs 1, 2, 3 amino acid sequences are set forth in SEQ ID NOs: 114, 115, 116, respectively;
(10) the selected VL CDRs 1, 2, 3 amino acid sequences are set forth in SEQ ID NOs: 117, 118, 119, respectively;
(11) the selected VL CDRs 1, 2, 3 amino acid sequences are set forth in SEQ ID NOs: 120, 121, 122, respectively;
(12) the selected VL CDRs 1, 2, 3 amino acid sequences are set forth in SEQ ID NOs: 123, 124, 125, respectively;
(13) the selected VL CDRs 1, 2, 3 amino acid sequences are set forth in SEQ ID NOs: 126, 127, 128, respectively;
(14) the selected VL CDRs 1, 2, 3 amino acid sequences are set forth in SEQ ID NOs: 129, 130, 131, respectively;
(15) the selected VL CDRs 1, 2, 3 amino acid sequences are set forth in SEQ ID NOs: 132, 133, 134, respectively;
(16) the selected VL CDRs 1, 2, 3 amino acid sequences are set forth in SEQ ID NOs: 135, 136, 137, respectively;
(17) the selected VL CDRs 1, 2, 3 amino acid sequences are set forth in SEQ ID NOs: 138, 139, 140, respectively;
(18) the selected VL CDRs 1, 2, 3 amino acid sequences are set forth in SEQ ID NOs: 141, 142, 143, respectively;
(19) the selected VL CDRs 1, 2, 3 amino acid sequences are set forth in SEQ ID NOs: 180, 181, 182, respectively;
(20) the selected VL CDRs 1, 2, 3 amino acid sequences are set forth in SEQ ID NOs: 183, 184, 185, respectively;
(21) the selected VL CDRs 1, 2, 3 amino acid sequences are set forth in SEQ ID NOs: 186, 187, 188, respectively;
(22) the selected VL CDRs 1, 2, 3 amino acid sequences are set forth in SEQ ID NOs: 189, 190, 191, respectively;
(23) the selected VL CDRs 1, 2, 3 amino acid sequences are set forth in SEQ ID NOs: 192, 193, 194, respectively;
(24) the selected VL CDRs 1, 2, 3 amino acid sequences are set forth in SEQ ID NOs: 195, 196, 197, respectively;
(25) the selected VL CDRs 1, 2, 3 amino acid sequences are set forth in SEQ ID NOs: 198, 199, 200, respectively;
(26) the selected VL CDRs 1, 2, 3 amino acid sequences are set forth in SEQ ID NOs: 201, 202, 203, respectively;
(27) the selected VL CDRs 1, 2, 3 amino acid sequences are set forth in SEQ ID NOs: 204, 205, 206, respectively;
(28) the selected VL CDRs 1, 2, 3 amino acid sequences are set forth in SEQ ID NOs: 207, 208, 209, respectively;
(29) the selected VL CDRs 1, 2, 3 amino acid sequences are set forth in SEQ ID NOs: 210, 211, 212, respectively; and
(30) the selected VL CDRs 1, 2, 3 amino acid sequences are set forth in SEQ ID NOs: 213, 214, 215, respectively.

In one aspect, the disclosure is related to an antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDR 1 is SYVMH (SEQ ID NO: 99); the selected VH CDR2 is VISYX₁X₂SDKYYADSVKG (SEQ ID NO: 100); the selected VH CDR3 is GRGYRYGQSYYYGMDV (SEQ ID NO: 101);
(2) the selected VH CDR 1 is SYVVH (SEQ ID NO: 102); the selected VH CDR2 is VISYX₁X₂DNKFYADSVKG (SEQ ID NO: 103); the selected VH CDR3 is GRX₅YX₆X₇YYGLDV (SEQ ID NO: 104);
(3) the selected VH CDR 1 is TYVMH (SEQ ID NO: 105); the selected VH CDR2 is VISYX₁X₂NNKYYADSVKG (SEQ ID NO: 106); the selected VH CDR3 is GRSYVNYYGLDV (SEQ ID NO: 107);
(4) the selected VH CDR 1 is GFSFSSY (SEQ ID NO: 171); the selected VH CDR2 is SYX₁X₂SD (SEQ ID NO: 172); the selected VH CDR3 is GRGYRYGQSYYYGMDV (SEQ ID NO: 173);
(5) the selected VH CDR 1 is GFTFSSY (SEQ ID NO: 174); the selected VH CDR2 is SYX₁X₂DN (SEQ ID NO: 175); the selected VH CDR3 is GRX₅YX₆X₇YYGLDV (SEQ ID NO: 176); and
(6) the selected VH CDR 1 is GFTFSTY (SEQ ID NO: 177); the selected VH CDR2 is SYX₁X₂NN (SEQ ID NO: 178); the selected VH CDR3 is GRSYVNYYGLDV (SEQ ID NO: 179);
wherein the selected VL CDRs 1, 2, and 3 amino acid sequences are one of the following:
(1) the selected VL CDR 1 is KSSQSLLYSX₃X₄KTYLY (SEQ ID NO: 135); the selected VL CDR2 is EVSNRFS (SEQ ID NO: 136); the selected VL CDR3 is MQSIKLPLT (SEQ ID NO: 137);
(2) the selected VL CDR 1 is KSSQSLLYSX₃X₄KTYLY (SEQ ID NO: 138); the selected VL CDR2 is EVSNRFS (SEQ ID NO: 139); the selected VL CDR3 is MQSLKVPPT (SEQ ID NO: 140);
(3) the selected VL CDR 1 is KSSQSLLYSX₃X₄KTYLY (SEQ ID NO: 141); the selected VL CDR2 is EVSNRFS (SEQ ID NO: 142); the selected VL CDR3 is MQSVKIPLT (SEQ ID NO: 143);
(4) the selected VL CDR 1 is KSSQSLLYSX₃X₄KTYLY (SEQ ID NO: 207); the selected VL CDR2 is EVSNRFS (SEQ ID NO: 208); the selected VL CDR3 is MQSIKLPLT (SEQ ID NO: 209);
(5) the selected VL CDR 1 is KSSQSLLYSX₃X₄KTYLY (SEQ ID NO: 210); the selected VL CDR2 is EVSNRFS (SEQ ID NO: 211); the selected VL CDR3 is MQSLKVPPT (SEQ ID NO: 212); and
(6) the selected VL CDR 1 is KSSQSLLYSX₃X₄KTYLY (SEQ ID NO: 213); the selected VL CDR2 is EVSNRFS (SEQ ID NO: 214); the selected VL CDR3 is MQSVKIPLT (SEQ ID NO: 215);
wherein X₁ and X₃ in each sequence are independently selected from the group consisting of D, S, and A,
wherein X₂ and X₄ in each sequence are independently selected from the group consisting of G and A,
wherein X₅ in SEQ ID NO: 104 or 176 is independently selected from the group consisting of N and S,
wherein X₆ in SEQ ID NO: 104 or 176 is independently selected from the group consisting of Y and V,
wherein X₇ in SEQ ID NO: 104 or 176 is independently selected from the group consisting of Y and N.

In some embodiments, the selected VH CDRs 1, 2, and 3 amino acid sequences and the selected VL CDRs, 1, 2, and 3 amino acid sequences are one of the following:
(1) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 19, 20 and 21, respectively according to Kabat definition;
(2) the VH comprises CDRs 1, 2, 3 with the amino acid sequences set forth in SEQ ID NOs: 4, 5 and 6, respectively, and the VL comprises CDRs 1, 2, 3 with the amino acid sequences set forth in SEQ ID NOs: 22, 23 and 24, respectively according to Kabat definition;
(3) the VH comprises CDRs 1, 2, 3 with the amino acid sequences set forth in SEQ ID NOs: 7, 8 and 9, respectively, and the VL comprises CDRs 1, 2, 3 with the amino acid sequences set forth in SEQ ID NOs: 25, 26 and 27, respectively according to Kabat definition;
(4) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 28, 29, and 30, respectively according to Chothia definition;
(5) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 31, 32, and 33, respectively according to Chothia definition;
(6) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 34, 35, and 36, respectively according to Chothia definition;
(7) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 22, 23 and 24, respectively according to Kabat definition;
(8) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 25, 26 and 27, respectively according to Kabat definition;
(9) the VH comprises CDRs 1, 2, 3 with the amino acid sequences set forth in SEQ ID NOs: 4, 5, and 6 respectively, and the VL comprises CDRs 1, 2, 3 with the amino acid sequences set forth in SEQ ID NOs: 19, 20 and 21, respectively according to Kabat definition;
(10) the VH comprises CDRs 1, 2, 3 with the amino acid sequences set forth in SEQ ID NOs: 4, 5, and 6 respectively, and the VL comprises CDRs 1, 2, 3 with the amino acid sequences set forth in SEQ ID NOs: 25, 26, and 27, respectively according to Kabat definition;
(11) the VH comprises CDRs 1, 2, 3 with the amino acid sequences set forth in SEQ ID NOs: 7, 8 and 9 respectively, and the VL comprises CDRs 1, 2, 3 with the amino acid sequences set forth in SEQ ID NOs: 19, 20 and 21, respectively according to Kabat definition;
(12) the VH comprises CDRs 1, 2, 3 with the amino acid sequences set forth in SEQ ID NOs: 7, 8, and 9 respectively, and the VL comprises CDRs 1, 2, 3 with the amino acid sequences set forth in SEQ ID NOs: 22, 23, and 24, respectively according to Kabat definition;
(13) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 31, 32, and 33, respectively according to Chothia definition;
(14) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 34, 35, and 36, respectively according to Chothia definition;
(15) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 28, 29, and 30, respectively according to Chothia definition;
(16) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 34, 35, and 36, respectively according to Chothia definition;
(17) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 28, 29, and 30, respectively according to Chothia definition; and
(18) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 31, 32, and 33, respectively according to Chothia definition.

In some embodiments, the selected VH CDRs 1, 2, and 3 amino acid sequences and the selected VL CDRs, 1, 2, and 3 amino acid sequences are one of the following:
(1) the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 114, 115, and 116, respectively;
(2) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 114, 115, and 116, respectively;
(3) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 108, 109, and 110, respectively;
(4) the VH comprises CDRs 1, 2, 3 with the amino acid sequences set forth in SEQ ID NOs: 78, 79, and 80, respectively, and the VL comprises CDRs 1, 2, 3 with the amino acid sequences set forth in SEQ ID NOs: 114, 115, and 116, respectively;
(5) the VH comprises CDRs 1, 2, 3 with the amino acid sequences set forth in SEQ ID NOs: 75, 76, and 77, respectively, and the VL comprises CDRs 1, 2, 3 with the amino acid sequences set forth in SEQ ID NOs: 111, 112, and 113, respectively;
(6) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 186, 187, and 188, respectively;
(7) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 186, 187, and 188, respectively;
(8) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 180, 181, and 182, respectively;
(9) the VH comprises CDRs 1, 2, 3 with the amino acid sequences set forth in SEQ ID NOs: 150, 151, and 152, respectively, and the VL comprises CDRs 1, 2, 3 with the amino acid sequences set forth in SEQ ID NOs: 186, 187, and 188, respectively;
(10) the VH comprises CDRs 1, 2, 3 with the amino acid sequences set forth in SEQ ID NOs: 147, 148, and 149, respectively, and the VL comprises CDRs 1, 2, 3 with the amino acid sequences set forth in SEQ ID NOs: 183, 184, and 185, respectively;
(11) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 25, 26, and 27, respectively;
(12) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 34, 35, and 36, respectively;
(13) the VH comprises CDRs 1, 2, 3 with the amino acid sequences set forth in SEQ ID NOs: 219, 220, and 221, respectively, and the VL comprises CDRs 1, 2, 3 with the amino acid sequences set forth in SEQ ID NOs: 22, 23, and 24, respectively;
(14) the VH comprises CDRs 1, 2, 3 with the amino acid sequences set forth in SEQ ID NOs: 222, 223, and 224, respectively, and the VL comprises CDRs 1, 2, 3 with the amino acid sequences set forth in SEQ ID NOs: 22, 23, and 24, respectively;
(15) the VH comprises CDRs 1, 2, 3 with the amino acid sequences set forth in SEQ ID NOs: 225, 226, and 227, respectively, and the VL comprises CDRs 1, 2, 3 with the amino acid sequences set forth in SEQ ID NOs: 22, 23, and 24, respectively;
(16) the VH comprises CDRs 1, 2, 3 with the amino acid sequences set forth in SEQ ID NOs: 228, 229, and 230, respectively, and the VL comprises CDRs 1, 2, 3 with the amino acid sequences set forth in SEQ ID NOs: 22, 23, and 24, respectively;
(17) the VH comprises CDRs 1, 2, 3 with the amino acid sequences set forth in SEQ ID NOs: 231, 232, and 233, respectively, and the VL comprises CDRs 1, 2, 3 with the amino acid sequences set forth in SEQ ID NOs: 22, 23, and 24, respectively;
(18) the VH comprises CDRs 1, 2, 3 with the amino acid sequences set forth in SEQ ID NOs: 234, 235, and 236, respectively, and the VL comprises CDRs 1, 2, 3 with the amino acid sequences set forth in SEQ ID NOs: 22, 23, and 24, respectively;
(19) the VH comprises CDRs 1, 2, 3 with the amino acid sequences set forth in SEQ ID NOs: 237, 238, and 239, respectively, and the VL comprises CDRs 1, 2, 3 with the amino acid sequences set forth in SEQ ID NOs: 22, 23, and 24, respectively;
(20) the VH comprises CDRs 1, 2, 3 with the amino acid sequences set forth in SEQ ID NOs: 240, 241, and 242, respectively, and the VL comprises CDRs 1, 2, 3 with the amino acid sequences set forth in SEQ ID NOs: 31, 32, and 33, respectively;
(21) the VH comprises CDRs 1, 2, 3 with the amino acid sequences set forth in SEQ ID NOs: 243, 244, and 245, respectively, and the VL comprises CDRs 1, 2, 3 with the amino acid sequences set forth in SEQ ID NOs: 31, 32, and 33, respectively;
(22) the VH comprises CDRs 1, 2, 3 with the amino acid sequences set forth in SEQ ID NOs: 246, 247, and 248, respectively, and the VL comprises CDRs 1, 2, 3 with the amino acid sequences set forth in SEQ ID NOs: 31, 32, and 33, respectively;
(23) the VH comprises CDRs 1, 2, 3 with the amino acid sequences set forth in SEQ ID NOs: 249, 250, and 251, respectively, and the VL comprises CDRs 1, 2, 3 with the amino acid sequences set forth in SEQ ID NOs: 31, 32, and 33, respectively;
(24) the VH comprises CDRs 1, 2, 3 with the amino acid sequences set forth in SEQ ID NOs: 252, 253, and 254, respectively, and the VL comprises CDRs 1, 2, 3 with the amino acid sequences set forth in SEQ ID NOs: 31, 32, and 33, respectively;
(25) the VH comprises CDRs 1, 2, 3 with the amino acid sequences set forth in SEQ ID NOs: 255, 256, and 257, respectively, and the VL comprises CDRs 1, 2, 3 with the amino acid sequences set forth in SEQ ID NOs: 31, 32, and 33, respectively; and
(26) the VH comprises CDRs 1, 2, 3 with the amino acid sequences set forth in SEQ ID NOs: 258, 259, and 260, respectively, and the VL comprises CDRs 1, 2, 3 with the amino acid sequences set forth in SEQ ID NOs: 31, 32, and 33, respectively.

In some embodiments, the antibody or antigen-binding fragment specifically binds to human CCR8, mouse CCR8 or monkey CCR8.

In some embodiments, the antibody or antigen-binding fragment is a human or humanized antibody or antigen-binding fragment thereof (e.g., a human IgG1 antibody).

In some embodiments, the antibody or antigen-binding fragment is a single-chain variable fragment (scFv), a one-armed antibody, or a multi-specific antibody (e.g., a bispecific antibody).

In one aspect, the disclosure is related to a nucleic acid comprising a polynucleotide encoding a polypeptide comprising: an immunoglobulin heavy chain or a fragment thereof comprising a heavy chain variable region (VH) comprising complementarity determining regions (CDRs) 1, 2, and 3, and wherein the VH, when paired with a light chain variable region (VL) binds to CCR8,
wherein the VH CDRs 1, 2, and 3 amino acid sequences are one of the following:
(1) the selected VH CDRs 1, 2, 3 amino acid sequences are set forth in SEQ ID NOs: 1, 2, 3, respectively,
(2) the selected VH CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively;
(4) the selected VH CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively;
(6) the selected VH CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 72, 73, 74, respectively;
(8) the selected VH CDRs 1, 2, 3 amino acid sequences are set forth in SEQ ID NOs: 75, 76, 77, respectively;
(9) the selected VH CDRs 1, 2, 3 amino acid sequences are set forth in SEQ ID NOs: 78, 79, 80, respectively;
(10) the selected VH CDRs 1, 2, 3 amino acid sequences are set forth in SEQ ID NOs: 81, 82, 83, respectively;
(11) the selected VH CDRs 1, 2, 3 amino acid sequences are set forth in SEQ ID NOs: 84, 85, 86, respectively;
(12) the selected VH CDRs 1, 2, 3 amino acid sequences are set forth in SEQ ID NOs: 87, 88, 89, respectively;
(13) the selected VH CDRs 1, 2, 3 amino acid sequences are set forth in SEQ ID NOs: 90, 91, 92, respectively;
(14) the selected VH CDRs 1, 2, 3 amino acid sequences are set forth in SEQ ID NOs: 93, 94, 95, respectively;
(15) the selected VH CDRs 1, 2, 3 amino acid sequences are set forth in SEQ ID NOs: 96, 97, 98, respectively;
(16) the selected VH CDRs 1, 2, 3 amino acid sequences are set forth in SEQ ID NOs: 99, 100, 101, respectively;
(17) the selected VH CDRs 1, 2, 3 amino acid sequences are set forth in SEQ ID NOs: 102, 103, 104, respectively;
(18) the selected VH CDRs 1, 2, 3 amino acid sequences are set forth in SEQ ID NOs: 105, 106, 107, respectively;
(19) the selected VH CDRs 1, 2, 3 amino acid sequences are set forth in SEQ ID NOs: 144, 145, 146, respectively;
(20) the selected VH CDRs 1, 2, 3 amino acid sequences are set forth in SEQ ID NOs: 147, 148, 149, respectively;
(21) the selected VH CDRs 1, 2, 3 amino acid sequences are set forth in SEQ ID NOs: 150, 151, 152, respectively;
(22) the selected VH CDRs 1, 2, 3 amino acid sequences are set forth in SEQ ID NOs: 153, 154, 155, respectively;
(23) the selected VH CDRs 1, 2, 3 amino acid sequences are set forth in SEQ ID NOs: 156, 157, 158, respectively;
(24) the selected VH CDRs 1, 2, 3 amino acid sequences are set forth in SEQ ID NOs: 159, 160, 161, respectively;
(25) the selected VH CDRs 1, 2, 3 amino acid sequences are set forth in SEQ ID NOs: 162, 163, 164, respectively;
(26) the selected VH CDRs 1, 2, 3 amino acid sequences are set forth in SEQ ID NOs: 165, 166, 167, respectively;
(27) the selected VH CDRs 1, 2, 3 amino acid sequences are set forth in SEQ ID NOs: 168, 169, 170, respectively;
(28) the selected VH CDRs 1, 2, 3 amino acid sequences are set forth in SEQ ID NOs: 171, 172, 173, respectively;
(29) the selected VH CDRs 1, 2, 3 amino acid sequences are set forth in SEQ ID NOs: 174, 175, 176, respectively;
(30) the selected VH CDRs 1, 2, 3 amino acid sequences are set forth in SEQ ID NOs: 177, 178, 179, respectively;
(31) the selected VH CDRs 1, 2, 3 amino acid sequences are set forth in SEQ ID NOs: 219, 220, 221, respectively;
(32) the selected VH CDRs 1, 2, 3 amino acid sequences are set forth in SEQ ID NOs: 222, 223, 224, respectively;
(33) the selected VH CDRs 1, 2, 3 amino acid sequences are set forth in SEQ ID NOs: 225, 226, 227, respectively;
(34) the selected VH CDRs 1, 2, 3 amino acid sequences are set forth in SEQ ID NOs: 228, 229, 230, respectively;
(35) the selected VH CDRs 1, 2, 3 amino acid sequences are set forth in SEQ ID NOs: 231, 232, 233, respectively;
(36) the selected VH CDRs 1, 2, 3 amino acid sequences are set forth in SEQ ID NOs: 234, 235, 236, respectively;
(37) the selected VH CDRs 1, 2, 3 amino acid sequences are set forth in SEQ ID NOs: 237, 238, 239, respectively;
(38) the selected VH CDRs 1, 2, 3 amino acid sequences are set forth in SEQ ID NOs: 240, 241, 242, respectively;
(39) the selected VH CDRs 1, 2, 3 amino acid sequences are set forth in SEQ ID NOs: 243, 244, 245, respectively;
(40) the selected VH CDRs 1, 2, 3 amino acid sequences are set forth in SEQ ID NOs: 246, 247, 248, respectively;
(41) the selected VH CDRs 1, 2, 3 amino acid sequences are set forth in SEQ ID NOs: 249, 250, 251, respectively;
(42) the selected VH CDRs 1, 2, 3 amino acid sequences are set forth in SEQ ID NOs: 252, 253, 254, respectively;
(43) the selected VH CDRs 1, 2, 3 amino acid sequences are set forth in SEQ ID NOs: 255, 256, 257, respectively; and
(44) the selected VH CDRs 1, 2, 3 amino acid sequences are set forth in SEQ ID NOs: 258, 259, 260, respectively;
wherein the VL amino acid sequence is selected from the group consisting of: SEQ ID NOs: 40, 41, 42, 66, 67, and 68.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4, 5 and 6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8 and 9, respectively.

In one aspect, the disclosure is related to a nucleic acid comprising a polynucleotide encoding a polypeptide comprising: an immunoglobulin heavy chain or a fragment thereof comprising a light chain variable region (VL) comprising complementarity determining regions (CDRs) 1, 2, and 3, and wherein the VL, when paired with a heavy chain variable region (VH) binds to CCR8,
wherein the selected VL CDRs, 1, 2, and 3 amino acid sequences are one of the following:
(1) the selected VL CDRs 1, 2, 3 amino acid sequences are set forth in SEQ ID NOs: 19, 20, 21, respectively;
(2) the selected VL CDRs 1, 2, 3 amino acid sequences are set forth in SEQ ID NOs: 22, 23, 24, respectively;
(3) the selected VL CDRs 1, 2, 3 amino acid sequences are set forth in SEQ ID NOs: 25, 26, 27, respectively;
(4) the selected VL CDRs 1, 2, 3 amino acid sequences are set forth in SEQ ID NOs: 28, 29, 30, respectively;
(5) the selected VL CDRs 1, 2, 3 amino acid sequences are set forth in SEQ ID NOs: 31, 32, 33, respectively;
(6) the selected VL CDRs 1, 2, 3 amino acid sequences are set forth in SEQ ID NOs: 34, 35, 36, respectively;
(7) the selected VL CDRs 1, 2, 3 amino acid sequences are set forth in SEQ ID NOs: 108, 109, 110, respectively;
(8) the selected VL CDRs 1, 2, 3 amino acid sequences are set forth in SEQ ID NOs: 111, 112, 113, respectively;
(9) the selected VL CDRs 1, 2, 3 amino acid sequences are set forth in SEQ ID NOs: 114, 115, 116, respectively;
(10) the selected VL CDRs 1, 2, 3 amino acid sequences are set forth in SEQ ID NOs: 117, 118, 119, respectively;
(11) the selected VL CDRs 1, 2, 3 amino acid sequences are set forth in SEQ ID NOs: 120, 121, 122, respectively;
(12) the selected VL CDRs 1, 2, 3 amino acid sequences are set forth in SEQ ID NOs: 123, 124, 125, respectively;
(13) the selected VL CDRs 1, 2, 3 amino acid sequences are set forth in SEQ ID NOs: 126, 127, 128, respectively;
(14) the selected VL CDRs 1, 2, 3 amino acid sequences are set forth in SEQ ID NOs: 129, 130, 131, respectively;
(15) the selected VL CDRs 1, 2, 3 amino acid sequences are set forth in SEQ ID NOs: 132, 133, 134, respectively;
(16) the selected VL CDRs 1, 2, 3 amino acid sequences are set forth in SEQ ID NOs: 135, 136, 137, respectively;
(17) the selected VL CDRs 1, 2, 3 amino acid sequences are set forth in SEQ ID NOs: 138, 139, 140, respectively;
(18) the selected VL CDRs 1, 2, 3 amino acid sequences are set forth in SEQ ID NOs: 141, 142, 143, respectively;
(19) the selected VL CDRs 1, 2, 3 amino acid sequences are set forth in SEQ ID NOs: 180, 181, 182, respectively;
(20) the selected VL CDRs 1, 2, 3 amino acid sequences are set forth in SEQ ID NOs: 183, 184, 185, respectively;
(21) the selected VL CDRs 1, 2, 3 amino acid sequences are set forth in SEQ ID NOs: 186, 187, 188, respectively;
(22) the selected VL CDRs 1, 2, 3 amino acid sequences are set forth in SEQ ID NOs: 189, 190, 191, respectively;
(23) the selected VL CDRs 1, 2, 3 amino acid sequences are set forth in SEQ ID NOs: 192, 193, 194, respectively;
(24) the selected VL CDRs 1, 2, 3 amino acid sequences are set forth in SEQ ID NOs: 195, 196, 197, respectively;
(25) the selected VL CDRs 1, 2, 3 amino acid sequences are set forth in SEQ ID NOs: 198, 199, 200, respectively;
(26) the selected VL CDRs 1, 2, 3 amino acid sequences are set forth in SEQ ID NOs: 201, 202, 203, respectively;
(27) the selected VL CDRs 1, 2, 3 amino acid sequences are set forth in SEQ ID NOs: 204, 205, 206, respectively;
(28) the selected VL CDRs 1, 2, 3 amino acid sequences are set forth in SEQ ID NOs: 207, 208, 209, respectively;
(29) the selected VL CDRs 1, 2, 3 amino acid sequences are set forth in SEQ ID NOs: 210, 211, 212, respectively; and
(30) the selected VL CDRs 1, 2, 3 amino acid sequences are set forth in SEQ ID NOs: 213, 214, 215, respectively,
wherein the VH amino acid sequence is selected from the group consisting of: SEQ ID NOs: 37, 38, 39, 63, 64, 65, 69, 70, 71, 216, 261, 262, 263, 264, 265, 266, and 267.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 19, 20 and 21, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 22, 23 and 24, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 25, 26 and 27, respectively.

In some embodiments, the VH when paired with a VL specifically binds to human CCR8, mouse CCR8 or monkey CCR8, or the VL when paired with a VH specifically binds to human CCR8, mouse CCR8 or monkey CCR8.

In some embodiments, the immunoglobulin heavy chain or the fragment thereof is a human or humanized immunoglobulin heavy chain or a fragment thereof, and the immunoglobulin light chain or the fragment thereof is a human or humanized immunoglobulin light chain or a fragment thereof.

In some embodiments, the nucleic acid encodes a single-chain variable fragment (scFv), a one-armed antibody, or a multi-specific antibody (e.g., a bispecific antibody).

In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VH region and the VL region that together bind to CCR8.

In one aspect, the disclosure is related to a pair of vectors, wherein each vector comprises one of the nucleic acids described herein, wherein together the pair of vectors encodes the VH region and the VL region that together bind to CCR8.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a cell comprising two of the nucleic acids described herein.

In some embodiments, the two nucleic acids together encode the VH region and the VL region that together bind to CCR8.

In one aspect, the disclosure is related to a method of producing an antibody or an antigen-binding fragment thereof, the method comprising:
(a) culturing the cell described herein under conditions sufficient for the cell to produce the antibody or the antigen-binding fragment; and
(b) collecting the antibody or the antigen-binding fragment produced by the cell.

In one aspect, the disclosure is related to an antibody or antigen-binding fragment thereof that binds to CCR8 comprising
a heavy chain variable region (VH) comprising an amino acid sequence that is at least 80% identical to a selected VH sequence, and a light chain variable region (VL) comprising an amino acid sequence that is at least 80% identical to a selected VL sequence,
wherein the selected VH sequence is selected from the group consisting of: SEQ ID NOs: 37, 38, 39, 63, 64, 65, 69, 70, 71, 216, 261, 262, 263, 264, 265, 266, and 267,
wherein the selected VL sequence is selected from the group consisting of: SEQ ID NOs: 40, 41, 42, 66, 67, and 68.

In some embodiments, the VH sequence and the VL sequence are one of the following:
(1) the VH comprises the sequence of SEQ ID NO: 37 and the VL comprises the sequence of SEQ ID NO: 40;
(2) the VH comprises the sequence of SEQ ID NO: 38 and the VL comprises the sequence of SEQ ID NO: 41;
(3) the VH comprises the sequence of SEQ ID NO: 39 and the VL comprises the sequence of SEQ ID NO: 42;
(4) the VH comprises the sequence of SEQ ID NO: 37 and the VL comprises the sequence of SEQ ID NO: 41;
(5) the VH comprises the sequence of SEQ ID NO: 37 and the VL comprises the sequence of SEQ ID NO: 42;
(6) the VH comprises the sequence of SEQ ID NO: 38 and the VL comprises the sequence of SEQ ID NO: 40;
(7) the VH comprises the sequence of SEQ ID NO: 38 and the VL comprises the sequence of SEQ ID NO: 42;
(8) the VH comprises the sequence of SEQ ID NO: 39 and the VL comprises the sequence of SEQ ID NO: 40;
(9) the VH comprises the sequence of SEQ ID NO: 39 and the VL comprises the sequence of SEQ ID NO: 41;
(10) the VH comprises the sequence of SEQ ID NO: 37 and the VL comprises the sequence of SEQ ID NO: 68;
(11) the VH comprises the sequence of SEQ ID NO: 63 and the VL comprises the sequence of SEQ ID NO: 68;
(12) the VH comprises the sequence of SEQ ID NO: 63 and the VL comprises the sequence of SEQ ID NO: 66;
(13) the VH comprises the sequence of SEQ ID NO: 65 and the VL comprises the sequence of SEQ ID NO: 68;
(14) the VH comprises the sequence of SEQ ID NO: 64 and the VL comprises the sequence of SEQ ID NO: 67;
(15) the VH comprises the sequence of SEQ ID NO: 63 and the VL comprises the sequence of SEQ ID NO: 42;
(16) the VH comprises the sequence of SEQ ID NO: 216 and the VL comprises the sequence of SEQ ID NO: 42;
(17) the VH comprises the sequence of SEQ ID NO: 261 and the VL comprises the sequence of SEQ ID NO: 41;
(18) the VH comprises the sequence of SEQ ID NO: 262 and the VL comprises the sequence of SEQ ID NO: 41;
(19) the VH comprises the sequence of SEQ ID NO: 263 and the VL comprises the sequence of SEQ ID NO: 41;
(20) the VH comprises the sequence of SEQ ID NO: 264 and the VL comprises the sequence of SEQ ID NO: 41;
(21) the VH comprises the sequence of SEQ ID NO: 265 and the VL comprises the sequence of SEQ ID NO: 41;
(22) the VH comprises the sequence of SEQ ID NO: 266 and the VL comprises the sequence of SEQ ID NO: 41; and
(23) the VH comprises the sequence of SEQ ID NO: 267 and the VL comprises the sequence of SEQ ID NO: 41.

In some embodiments, the antibody or antigen-binding fragment specifically binds to human CCR8, mouse CCR8 or monkey CCR8.

In some embodiments, the antibody or antigen-binding fragment is a human or humanized antibody or antigen-binding fragment thereof.

In some embodiments, the antibody or antigen-binding fragment is a single-chain variable fragment (scFv), a one-armed antibody, or a multi-specific antibody (e.g., a bispecific antibody).

In one aspect, the disclosure is related to an antibody or antigen-binding fragment thereof that binds to CCR8 comprising a heavy chain variable region (VH) comprising VH CDRs 1, 2, 3, and a light chain variable region comprising VL CDRs 1, 2, 3, wherein the VH CDRs 1, 2, 3 and the VL CDRs 1, 2, 3 are identical to complementarity determining regions in the antibody or antigen-binding fragment thereof described herein.

In some embodiments, the VH CDRs 1, 2, 3 are identical to complementarity determining regions in SEQ ID NO: 37, 38, 39, 63, 64, 65, 69, 70, 71, 216, 261, 262, 263, 264, 265, 266, or 267, and the VL CDRs 1, 2, 3 are identical to complementary determining regions in SEQ ID NO: 40, 41, 42, 66, 67, or 68.

In one aspect, the disclosure is related to an antibody or antigen-binding fragment thereof that cross-competes with the antibody or antigen-binding fragment thereof described herein.

In one aspect, the disclosure is related to an antibody-drug conjugate comprising the antibody or antigen-binding fragment thereof described herein covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, or the antibody-drug conjugate described herein, to the subject.

In some embodiments, the subject has a solid tumor.

In some embodiments, the cancer is breast cancer, melanoma, non-small cell lung cancer, lung adenocarcinoma, colon cancer, or colorectal cancer (e.g., colorectal adenocarcinoma).

In some embodiments, the cancer is resistant to a PD-1 pathway inhibitor treatment.

In some embodiments, the method further comprises administering a PD-1 pathway inhibitor to the subject.

In some embodiments, the PD-1 pathway inhibitor is an anti-PD-1 antibody (e.g., Pembrolizumab, Nivolumab, or Cemiplimab).

In some embodiments, the PD-1 pathway inhibitor is an anti-PD-L1 antibody (e.g., Atezolizumab, Avelumab, or Durvalumab).

In one aspect, the disclosure is related to a method of decreasing the rate of tumor growth, the method comprising contacting a tumor cell with an effective amount of a composition comprising an antibody or antigen-binding fragment thereof described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, or the antibody-drug conjugate described herein.

In one aspect, the disclosure is related to a pharmaceutical composition comprising the antibody or antigen-binding fragment thereof of described herein, and a pharmaceutically acceptable carrier.

In one aspect, the disclosure is related to a pharmaceutical composition comprising the antibody drug conjugate described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fe region of a human antibody. The term antibody also includes derivatives, e.g., bi-specific antibodies, single-chain antibodies, heavy chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, VHH, and Fv fragments.

As used herein, the term “human antibody” refers to an antibody that is encoded by an endogenous nucleic acid (e.g., rearranged human immunoglobulin heavy or light chain locus) derived from a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different species (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., CCR8) to at least one second target molecule (e.g., PD-L1) on the surface of mammalian cell (e.g., Treg cell and cancer cell).

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., CCR8) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a CCR8 molecule may be referred to as a CCR8-specific antibody or an anti-CCR8 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the effects on tumor growth of the anti-CCR8 antibodies in vivo in a colon cancer model.

FIGS. 2A-2C show CDR sequences corresponding to anti-human CCR8 mAbs 11B12, 11F5, and 11F8 and CDR-modified anti-human CCR8 mAbs, as defined by Kabat definition.

FIGS. 3A-3C show CDR sequences corresponding to anti-human CCR8 mAbs 11B12, 1 1F5, and 11F8 and CDR-modified anti-human CCR8 mAbs, as defined by Chothia definition.

FIG. 4 shows some of the relevant amino acid sequences discussed in the present disclosure.

FIG. 5 shows some of the VH and VL combinations in the present disclosure.

FIG. 6 shows the average tumor volume in different groups of B-hPD-1/hPD-L1/hCCR8 mice that were subcutaneously injected with MC38 cells (murine colon cancer cells), and were treated with PBS (G1), Pembrolizumab analog (G2), anti-CCR8 antibody 11F5 (G3), or a combination of Pembrolizumab analog and 11F5 (G4).

FIG. 7 shows the average tumor volume in different groups of B-hPD-1/hPD-L1/hCCR8 mice that were subcutaneously injected with MC38 cells, and were treated with PBS (G1), Pembrolizumab analog (G2), anti-CCR8 antibody 1 1F5-SI (G3), a combination of Pembrolizumab analog and 11F5-SI (G4), anti-CCR8 antibody 11B12-SI (G5), or a combination of Pembrolizumab analog and 11B12-SI (G6).

FIG. 8 shows the blockade of hCCL1 binding to hCCR8 by anti-CCR8 antibodies 11 °F5 and 11F5's CDR3-modified antibodies, as tested by calcium (Ca) flux assays using human CCR8-expressing 293T cells.

### DETAILED DESCRIPTION

Chemokines and chemokine receptors play an important role in the immune defense system by controlling the migration, activation, differentiation, and survival of leukocytes. The 50 human chemokines are divided into C, CC, CXC, and CX3C classes based on the number and spacing of conserved cysteine residues in their N-terminus region. Chemokine receptors belong to the family A of G-protein coupled receptors (GPCRs), characterized by a seven transmembrane (7TM) helical domain. There are 18 human chemokine receptors that are primarily activated by different subfamilies of chemokines: C (XCR1), CC (CCR1, CCR2, CCR3, CCR4, CCR5, CCR6, CCR7, CCR8, CCR9, CCR10), CXC (CXCR1, CXCR2, CXCR3, CXCR4, CXCR5, CXCR6), or CX3C (CX3CR1), and four atypical decoy chemokine receptors (ACKRs: ACKR1, ACKR2, ACKR3/CXCR7, and ACKR4). Chemokine receptors are considered to interact with their chemokine ligands via a two-step binding mechanism in which: (i) the structured C-terminal region of the chemokine first binds the N-terminus region and extracellular loops (ECLs) of the receptor (chemokine recognition site 1, CRS1), allowing (ii) the unstructured N-terminus of the chemokine to target the 7TM helical bundle (chemokine recognition site 2, CRS2) and stabilize the receptor in an active conformation that facilitates intracellular signal transduction by, e.g., G-proteins or arrestins. Because of their crucial role in cell migration chemokine receptors are important therapeutic targets for inflammatory diseases and cancer.

Chemokine (C-C motif) receptor 8 (CCR8, or CDw198) is a seven transmembrane G protein-couple receptor (GPCR), and belongs to the C-C subfamily of chemokine receptors. CCR8 is one of the most important chemokine receptors and is mainly expressed in Tregs with small proportions of expression in T helper 2 cells and monocytes. CCR8 has four known ligands: CCL1, CCLX, CCL16 and CCL18. Human CCL1 is the dominant ligand for human CCR8 that binds simultaneously to the extracellular loop 2 and the N-terminal domain of the receptor. Specifically, CCL1 enhances Treg immunosuppressive activity by recruiting CCR8, FOXp3 and IL-10 with a positive feedback mechanism. CCR8 was also reported to be correlated with poor prognosis in non-small cell lung cancer and colorectal cancer.

CCR8 is selectively up-regulated in tumor resident Treg cells in multiple solid tumors, but rarely observed on Treg cells in peripheral blood mononuclear cells (PBMC) or normal organs. Treg cells are immunosuppressive and generally suppress or downregulate induction and proliferation of effector T cells. These cells play an important role in maintaining self-tolerance and immune homeostasis. Tumor-resident Treg cells can suppress anti-tumor immunity in the tumor microenvironment. High CCR8 expression is often associated with poor survival in cancer patients. Thus, CCR8 is a potential target for Treg cells. Treg cells inhibition and/or depletion by targeting CCR8 have several advantages. First, the expression of CCR8 is more restricted to the Treg cells, not on the CD8 T cells, there is no attenuation of T effector cell antitumor activities. Second, systemic Treg cell depletion can often lead to autoimmunity. There is less risk of autoimmunity as CCRX is rarely expressed on Treg cells in peripheral blood mononuclear cells (PBMC) or normal organs. In fact, treatment with rat anti-mouse CCR8 blocking antibodies can generate significant antitumor activity and improve long-term survival in a colorectal tumor mouse model. Therefore, CCR8 is a superior target among various known Treg targets as its expression is found to be highly selected in tumor-resident Treg cells. Anti-CCR8 antibodies can also have single agent activity in a PD-1 resistant setting and can restore PD-1 activity as a combination partner.

A detailed description of CCR8 and its function can be found, e.g., in Plitas G. et al., “Regulatory T cells exhibit distinct features in human breast cancer.” Immunity 45.5 (2016): 1122-1134; Villarreal. et al., “Targeting CCR8 induces protective antitumor immunity and enhances vaccine-induced responses in colon cancer.” Cancer research 78.18 (2018): 5340-5348; Arimont et al., “Structural analysis of chemokine receptor-ligand interactions.” Journal of Medicinal Chemistry 60.12 (2017): 4735-4779; Li et al., “Clinical and prognostic significance of CC chemokine receptor type 8 protein expression in gastrointestinal stromal tumors.” World Journal of Gastroenterology 26.31 (2020): 4656; each of which is incorporated by reference in its entirety.

The present disclosure provides anti-CCR8 antibodies, antigen-binding fragments thereof, and methods of using these anti-CCR8 antibodies and antigen-binding fragments to inhibit tumor growth and to treat cancers, particularly, the present disclosure provides full human anti-CCR8 antibodies (e.g., that are produced by mice with a humanized heavy chain immunoglobulin locus and a humanized light chain immunoglobulin locus).

### Anti-CCR8 Antibodies and Antigen-Binding Fragments

The disclosure provides antibodies and antigen-binding fragments thereof that specifically bind to CCR8. The antibodies and antigen-binding fragments described herein are capable of binding to CCR8. In some embodiments, these antibodies can block the binding of CCL1 to CCR8 thereby inhibiting CCL1-induced suppressive function of tumor resident Treg cells. In some embodiments, these antibodies can inhibit CCL1-CCR8-mediated downstream signaling. In some embodiments, these antibodies can initiate complement-dependent cytotoxicity (CMC) or antibody-dependent cellular cytotoxicity (ADCC). In some embodiments, these antibodies and antigen-binding fragments thereof can block the interaction between CCR8 and a CCR8 ligand (e.g., CCL1, CCL8, CCL16 or CCL18).

The disclosure provides e.g., mouse anti-CCR8 antibodies 11B12, 11F5, and 11F8, the chimeric antibodies thereof, and the humanized antibodies thereof.

The CDR sequences for 11B12, and 11B12 derived antibodies (e.g., humanized antibodies) include CDRs of the heavy chain variable domain, SEQ ID NOs: 1, 2, 3, and CDRs of the light chain variable domain, SEQ ID NOs: 19, 20, 21 as defined by Kabat definition. The CDRs can also be defined by Chothia definition. Under the Chothia definition, the CDR sequences of the heavy chain variable domain are set forth in SEQ ID NOs: 10, 11, 12, and CDR sequences of the light chain variable domain are set forth in SEQ ID NOs: 28, 29, 30.

Similarly, the CDR sequences for 11F5, and 11F5 derived antibodies include CDRs of the heavy chain variable domain, SEQ ID NOs: 4, 5, 6, and CDRs of the light chain variable domain, SEQ ID NOs: 22, 23, 24, as defined by Kabat definition. Under Chothia definition, the CDR sequences of the heavy chain variable domain are set forth in SEQ ID NOs: 13, 14, 15, and CDRs of the light chain variable domain are set forth in SEQ ID NOs: 31, 32, 33.

Similarly, the CDR sequences for 11F8, and 11F8 derived antibodies include CDRs of the heavy chain variable domain, SEQ ID NOs: 7, 8, 9, and CDRs of the light chain variable domain, SEQ ID NOs: 25, 26, 27, as defined by Kabat definition. Under Chothia definition, the CDR sequences of the heavy chain variable domain are set forth in SEQ ID NOs: 16, 17, 18, and CDRs of the light chain variable domain are set forth in SEQ ID NOs: 34, 35, 36.

The amino acid sequence for the heavy chain variable region of 11B12 antibody is set forth in SEQ ID NO: 37. The amino acid sequence for the light chain variable region of 11B12 antibody is set forth in SEQ ID NO: 40. The amino acid sequence for the heavy chain variable region of 11F5 antibody is set forth in SEQ ID NO: 38. The amino acid sequence for the light chain variable region of 11F5 antibody is set forth in SEQ ID NO: 41. The amino acid sequence for the heavy chain variable region of 11F8 antibody is set forth in SEQ ID NO: 39. The amino acid sequence for the light chain variable region of 11F8 antibody is set forth in SEQ ID NO: 42.

The amino acid sequences for heavy chain variable regions and light chain variable regions of the humanized antibodies are also provided. As there are different ways to humanize a mouse antibody (e.g., a sequence can be modified with different amino acid substitutions), the heavy chain and the light chain of an antibody can have more than one version of humanized sequences. In some embodiments, the humanized heavy chain variable region is at least 80%, 85%, 90%, 91%, 92%, 93%, 94%, 95%, 96%, 97%, 98%, or 99% identical to SEQ ID NO: 37, 38, or 39. In some embodiments, the humanized light chain variable region is at least 80%, 85%, 90%, 91%, 92%, 93% 94% 95% 96%, 97% 98%, or 99% identical to SEQ ID NO: 40, 41, or 42. The heavy chain variable region sequence can be paired with the corresponding light chain variable region sequence, and together they bind to CCR8.

Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the antibody is a full human antibody.

Furthermore, in some embodiments, the antibodies or antigen-binding fragments thereof described herein can also contain one, two, or three heavy chain variable region CDRs selected from the group of SEQ ID NOs: 1-3, SEQ ID NOs: 4-6, SEQ ID NOs: 7-9, SEQ ID NOs: 10-12, SEQ ID NOs: 13-15, and SEQ ID NOs: 16-18; and/or one, two, or three light chain variable region CDRs selected from the group of SEQ ID NOs: 19-21, SEQ ID NOs: 22-24, SEQ ID NOs: 25-27, SEQ ID NOs: 28-30, SEQ ID NOs: 31-33, and SEQ ID NOs: 34-36.

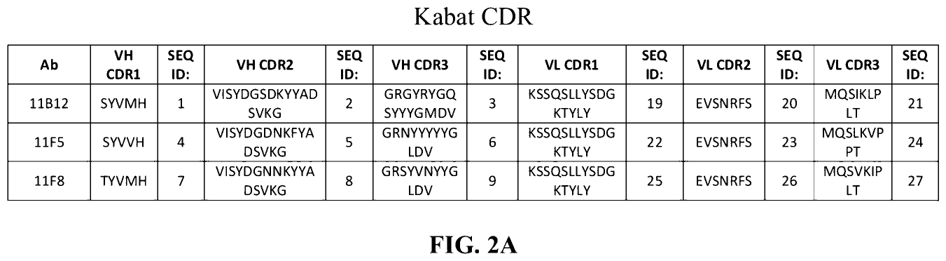

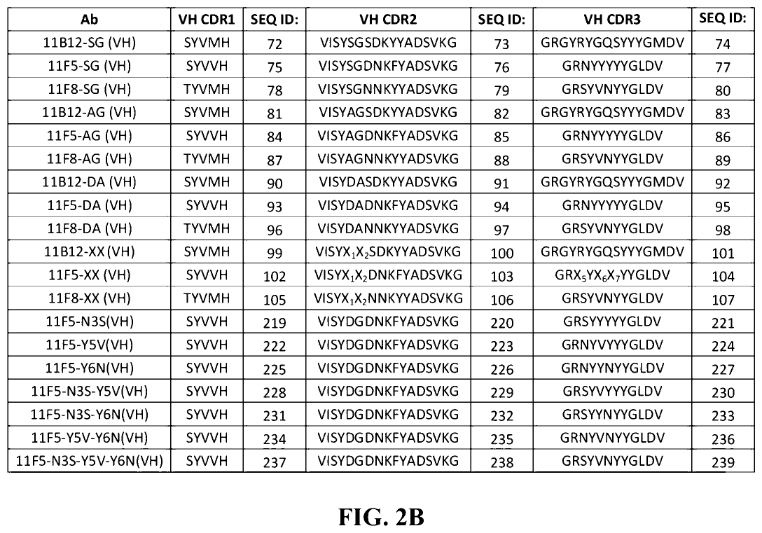

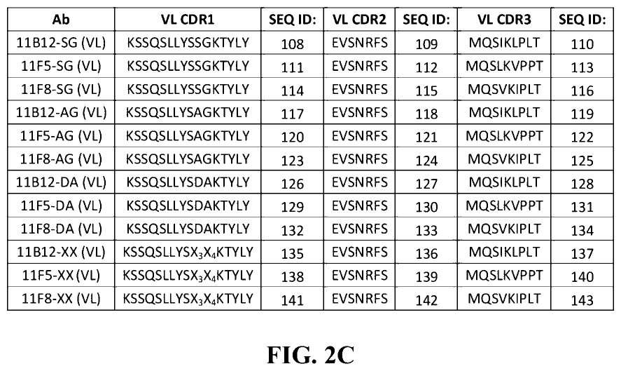

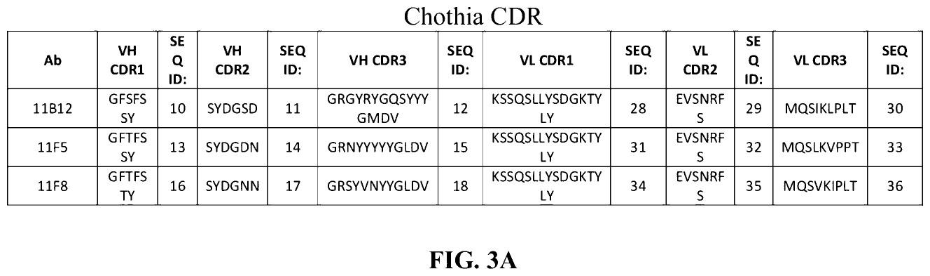

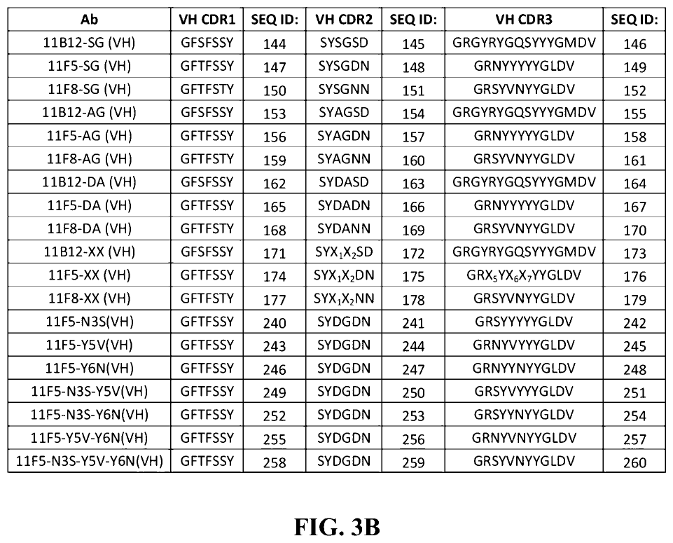

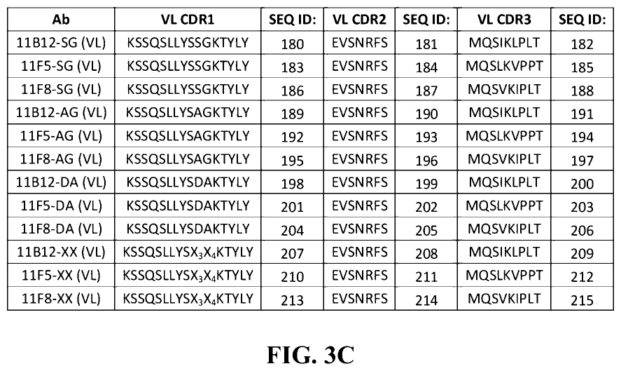

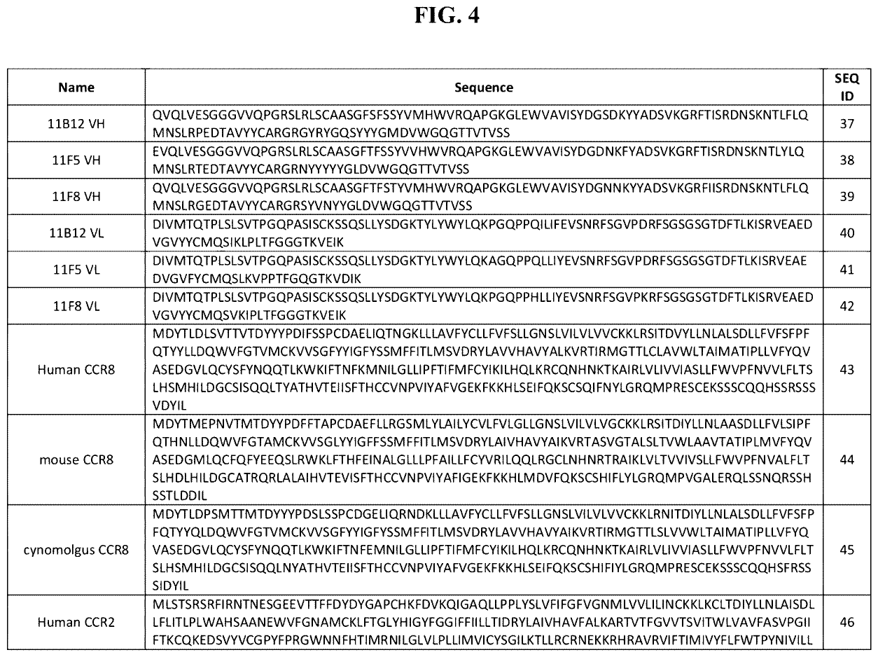

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIGS. 2A-2C (Kabat CDR) and FIGS. 3A-3C (Chothia CDR).

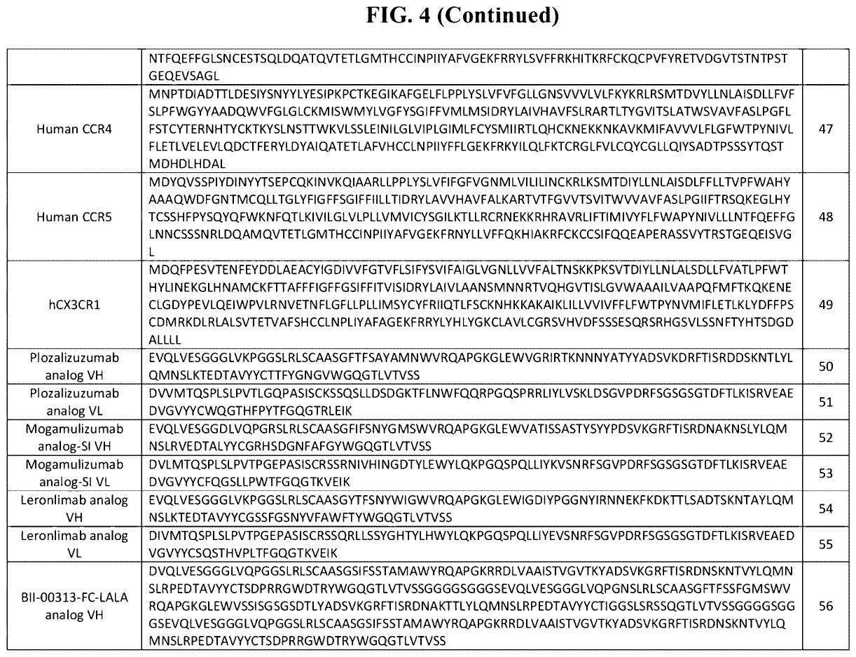

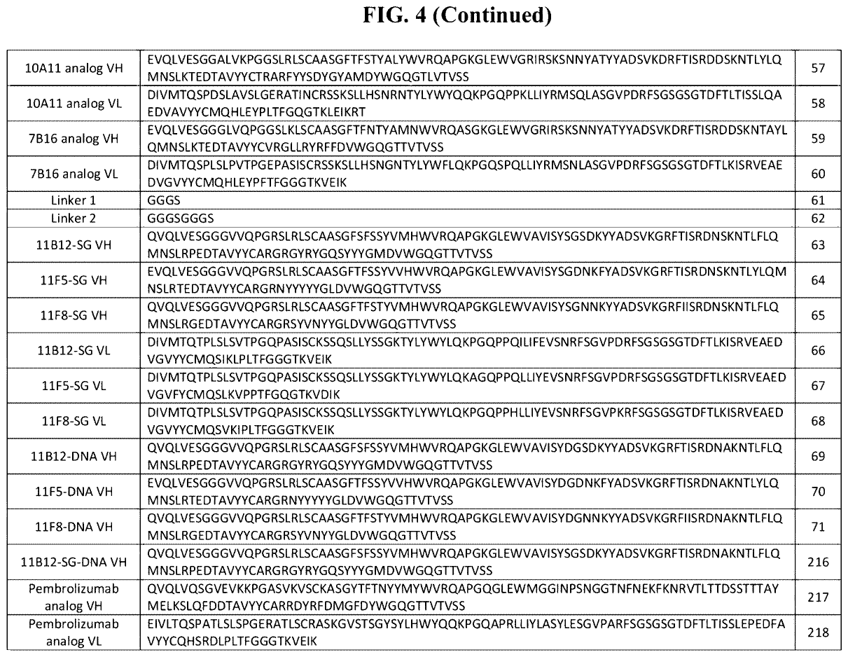

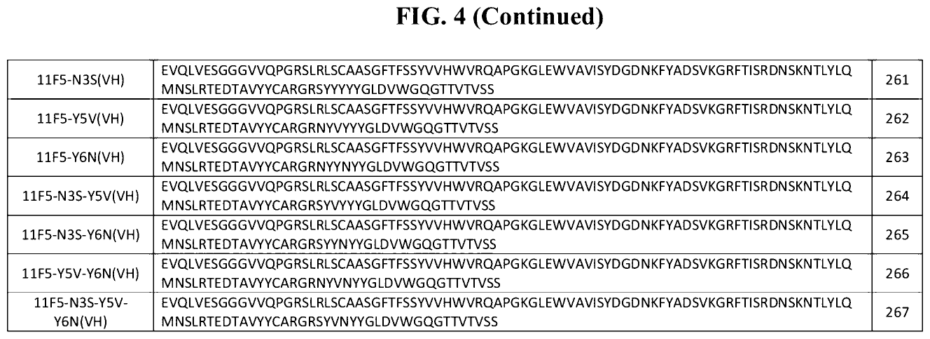

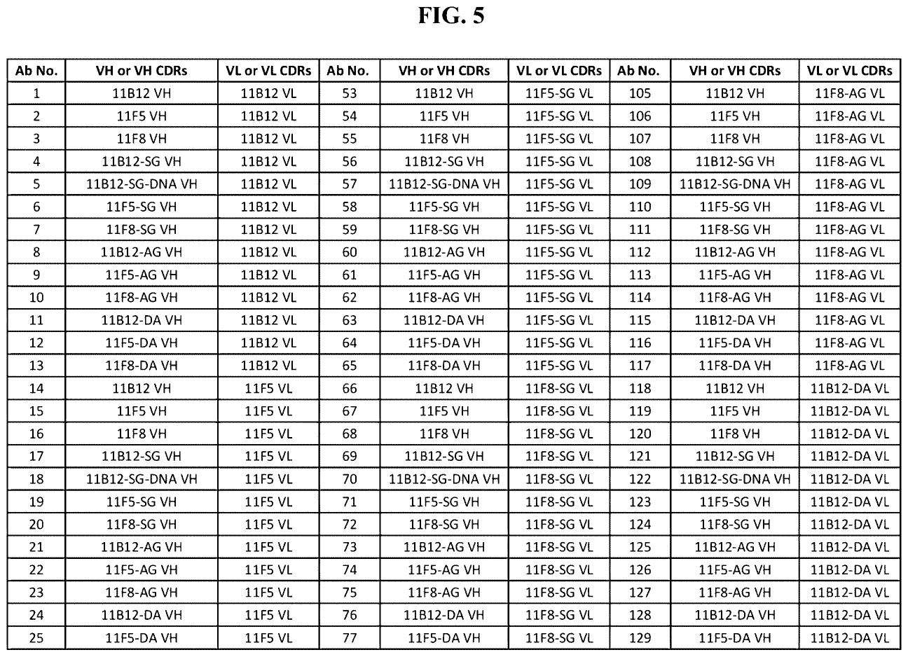

In some embodiments, the disclosure relates to CDR-modified VH or VL. In some embodiments, the “DG” in VH CDR2 of an antibody described herein is mutated to “SG,” “AG,” or “DA,” to create CDR-modified VH. For example, in one embodiment, the “DG” in VH CDR2 of 11B12 is mutated to “SG,” and the resulting CDR-modified VH is named 11B12-SG VH. The sequences of some of these CDR-modified VH can be found in FIG. 4.

In some embodiments, the “DG” in VL CDR1 of an antibody described herein is mutated to “SG,” “AQ” or “DA,” to create CDR-modified VL. For example, in one embodiment, the “DG” in VL CDR1 of 11B12 is mutated to SG, and the resulting CDR-modified VL is named 11B12-SG VL. The sequences of some of these CDR-modified VL can be found in FIG. 4.

In some embodiments, the disclosure relates to framework region modified (FR-modified) VH. In some embodiments, the “DNS” in the framework of the VH of an antibody described herein is mutated to “DNA,” to create FR-modified VH. For example, in one example, the “DNS” in the framework of the VH of 11B12 is mutated to “DNA,” and the resulting FR-modified VH is named 11B12-DNA VH. The sequences of some these FR-modified VH can be found in FIG. 4 (e.g., SEQ ID NOs: 69-71). Thus, in some embodiments, the antibody has a S74A mutation (Kabat numbering). In some embodiments, the amino acid at position 74 (Kabat numbering) is A.

In some embodiments, the VH has both a FR modification (e.g., DNA) and a CDR modification (e.g., SG). In one example, the “DNS” in the framework of the VH of 11B12 is mutated to “DNA,” and the “DG” in VH CDR2 of 11B12 is mutated to “SG,” and the resulting FR-modified and CDR-modified VH is named 11B12-SG-DNA VH.

In some embodiments, 11F5 CDR3-modified VHs can be created. In some embodiments, the amino acid “N” at position 3 in VH CDR3 of 11F5 (e.g., SEQ ID NO: 6 or 15) is mutated to “S.” In some embodiments, the amino acid “Y” at position 5 in VH CDR3 of 11F5 (e.g., SEQ ID NO: 6 or 15) is mutated to “V.” In some embodiments, the amino acid “Y” at position 6 in VH CDR3 of 11F5 (e.g., SEQ ID NO: 6 or 15) is mutated to “N.” For example, the amino acid “N” at position 3 in VH CDR3 of 11F5 can be mutated to “S” and the resulting CDR-modified VH is named 11F5-N3S VH; the amino acid “Y” at position 5 in VH CDR3 of 11F5 can be mutated to “V” and the resulting CDR-modified VH is named 11F5-Y5V VH; the amino acid “Y” at position 6 in VH CDR3 of 11F5 can be mutated to “N” and the resulting CDR-modified VH is named 11F5-Y6N VH; the amino acid “N” at position 3 and amino acid “Y” at position 5 in VH CDR3 of 11F5 can be mutated to “S” and “V,” respectively, and the resulting CDR-modified VH is named 11F5-N3S-Y5V VH; the amino acid “N” at position 3 and the amino acid “Y” at position 6 in VH CDR3 of 11F5 can be mutated to “S” and “N,” respectively, and the resulting CDR-modified VH is named 11F5-N3S-Y6N VH; the amino acid “Y” at position 5 and the amino acid “Y” at position 6 in VH CDR3 of the 11F5 can be mutated to “V” and “N,” respectively, and the resulting CDR-modified VH is named 11F5-Y5V-Y6N VH; the amino acid “N” at position 3, amino acid “Y” at position 5, and the amino acid “Y” at position 6 in VH CDR3 of the 11F5 can be mutated to “S,” “V,” and “N,” respectively, and the resulting CDR-modified VH is named 11F5-N3S-Y5V-Y6N VH. The sequences of these CDR-modified VHs can be found in FIG. 4.

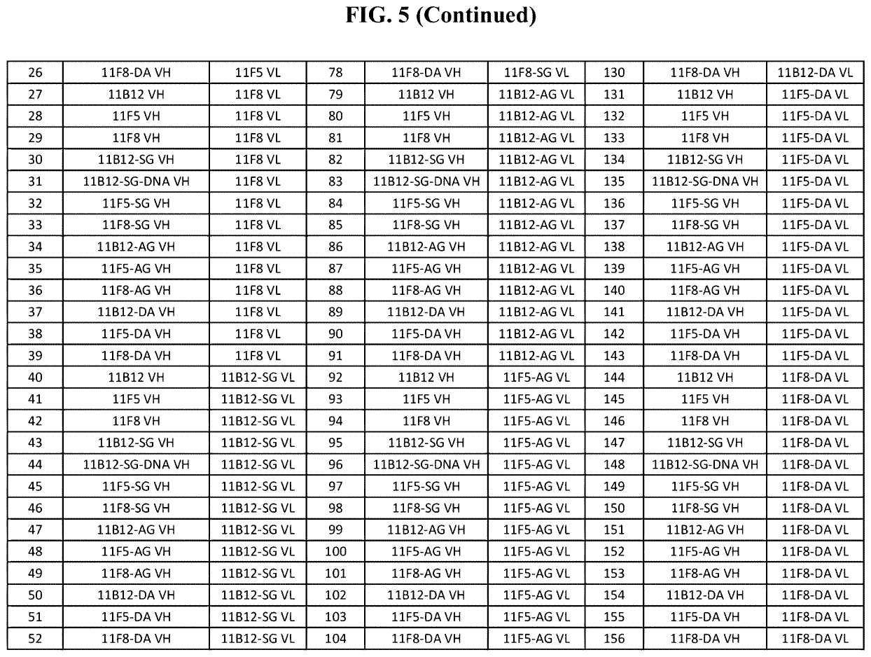

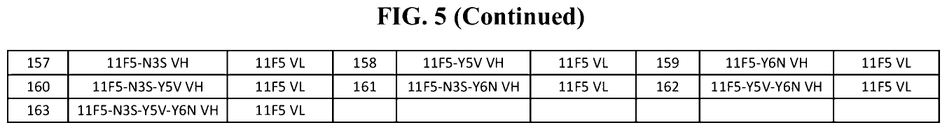

In some embodiments, the VH of any antibody described herein can be paired with the VL of any antibody described herein to form antibodies. For example, in one embodiment, the VH of 11B12 is paired with the VL of 11F8, and the resulting antibody is named 11B12-11F8. In one embodiment, the VH of 11B12-SG is paired with the VL of 11F8-SG, and the resulting antibody is named 11B12-SG-11F8-SG. In one embodiment, the VH of 11B12-SG is paired with the VL of 11B12-SG, and the resulting antibody is named 11B12-SG-11B12-SG. The binding affinities of some of these antibodies are shown in Tables 15, 16, and 19. Some of the disclosed VH and VL combinations are listed in FIG. 5. As shown in FIG. 5, at least 163 VH and VL combinations are disclosed. In addition, the VH CDRs and VL CDRs in the VH and VL can be combined as well, as shown in FIG. 5.

In one aspect, the disclosure is related to an antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a selected VH CDR1 amino acid sequence, the VH CDR2 region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a selected VL CDR1 amino acid sequence, the VL CDR2 region comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDRs 1, 2, 3 amino acid sequences are set forth in SEQ ID NOs: 1, 2, 3, respectively,
(2) the selected VH CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively;
(4) the selected VH CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively;
(6) the selected VH CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 72, 73, 74, respectively;
(8) the selected VH CDRs 1, 2, 3 amino acid sequences are set forth in SEQ ID NOs: 75, 76, 77, respectively;
(9) the selected VH CDRs 1, 2, 3 amino acid sequences are set forth in SEQ ID NOs: 78, 79, 80, respectively;
(10) the selected VH CDRs 1, 2, 3 amino acid sequences are set forth in SEQ ID NOs: 81, 82, 83, respectively;
(11) the selected VH CDRs 1, 2, 3 amino acid sequences are set forth in SEQ ID NOs: 84, 85, 86, respectively;
(12) the selected VH CDRs 1, 2, 3 amino acid sequences are set forth in SEQ ID NOs: 87, 88, 89, respectively;
(13) the selected VH CDRs 1, 2, 3 amino acid sequences are set forth in SEQ ID NOs: 90, 91, 92, respectively;
(14) the selected VH CDRs 1, 2, 3 amino acid sequences are set forth in SEQ ID NOs: 93, 94, 95, respectively;
(15) the selected VH CDRs 1, 2, 3 amino acid sequences are set forth in SEQ ID NOs: 96, 97, 98, respectively;
(16) the selected VH CDRs 1, 2, 3 amino acid sequences are set forth in SEQ ID NOs: 99, 100, 101, respectively;
(17) the selected VH CDRs 1, 2, 3 amino acid sequences are set forth in SEQ ID NOs: 102, 103, 104, respectively;
(18) the selected VH CDRs 1, 2, 3 amino acid sequences are set forth in SEQ ID NOs: 105, 106, 107, respectively;
(19) the selected VH CDRs 1, 2, 3 amino acid sequences are set forth in SEQ ID NOs: 144, 145, 146, respectively;
(20) the selected VH CDRs 1, 2, 3 amino acid sequences are set forth in SEQ ID NOs: 147, 148, 149, respectively;
(21) the selected VH CDRs 1, 2, 3 amino acid sequences are set forth in SEQ ID NOs: 150, 151, 152, respectively;
(22) the selected VH CDRs 1, 2, 3 amino acid sequences are set forth in SEQ ID NOs: 153, 154, 155, respectively;
(23) the selected VH CDRs 1, 2, 3 amino acid sequences are set forth in SEQ ID NOs: 156, 157, 158, respectively;
(24) the selected VH CDRs 1, 2, 3 amino acid sequences are set forth in SEQ ID NOs: 159, 160, 161, respectively;
(25) the selected VH CDRs 1, 2, 3 amino acid sequences are set forth in SEQ ID NOs: 162, 163, 164, respectively;
(26) the selected VH CDRs 1, 2, 3 amino acid sequences are set forth in SEQ ID NOs: 165, 166, 167, respectively;
(27) the selected VH CDRs 1, 2, 3 amino acid sequences are set forth in SEQ ID NOs: 168, 169, 170, respectively;
(28) the selected VH CDRs 1, 2, 3 amino acid sequences are set forth in SEQ ID NOs: 171, 172, 173, respectively;
(29) the selected VH CDRs 1, 2, 3 amino acid sequences are set forth in SEQ ID NOs: 174, 175, 176, respectively;
(30) the selected VH CDRs 1, 2, 3 amino acid sequences are set forth in SEQ ID NOs: 177, 178, 179, respectively;
(31) the selected VH CDRs 1, 2, 3 amino acid sequences are set forth in SEQ ID NOs: 219, 220, 221, respectively;
(32) the selected VH CDRs 1, 2, 3 amino acid sequences are set forth in SEQ ID NOs: 222, 223, 224, respectively;
(33) the selected VH CDRs 1, 2, 3 amino acid sequences are set forth in SEQ ID NOs: 225, 226, 227, respectively;
(34) the selected VH CDRs 1, 2, 3 amino acid sequences are set forth in SEQ ID NOs: 228, 229, 230, respectively;
(35) the selected VH CDRs 1, 2, 3 amino acid sequences are set forth in SEQ ID NOs: 231, 232, 233, respectively;
(36) the selected VH CDRs 1, 2, 3 amino acid sequences are set forth in SEQ ID NOs: 234, 235, 236, respectively;
(37) the selected VH CDRs 1, 2, 3 amino acid sequences are set forth in SEQ ID NOs: 237, 238, 239, respectively;
(38) the selected VH CDRs 1, 2, 3 amino acid sequences are set forth in SEQ ID NOs: 240, 241, 242, respectively;
(39) the selected VH CDRs 1, 2, 3 amino acid sequences are set forth in SEQ ID NOs: 243, 244, 245, respectively;
(40) the selected VH CDRs 1, 2, 3 amino acid sequences are set forth in SEQ ID NOs: 246, 247, 248, respectively;
(41) the selected VH CDRs 1, 2, 3 amino acid sequences are set forth in SEQ ID NOs: 249, 250, 251, respectively;
(42) the selected VH CDRs 1, 2, 3 amino acid sequences are set forth in SEQ ID NOs: 252, 253, 254, respectively;
(43) the selected VH CDRs 1, 2, 3 amino acid sequences are set forth in SEQ ID NOs: 255, 256, 257, respectively; and
(44) the selected VH CDRs 1, 2, 3 amino acid sequences are set forth in SEQ ID NOs: 258, 259, 260, respectively;
wherein the selected VL CDRs, 1, 2, and 3 amino acid sequences are one of the following:
(1) the selected VL CDRs 1, 2, 3 amino acid sequences are set forth in SEQ ID NOs: 19, 20, 21, respectively;
(2) the selected VL CDRs 1, 2, 3 amino acid sequences are set forth in SEQ ID NOs: 22, 23, 24, respectively;
(3) the selected VL CDRs 1, 2, 3 amino acid sequences are set forth in SEQ ID NOs: 25, 26, 27, respectively;
(4) the selected VL CDRs 1, 2, 3 amino acid sequences are set forth in SEQ ID NOs: 28, 29, 30, respectively;
(5) the selected VL CDRs 1, 2, 3 amino acid sequences are set forth in SEQ ID NOs: 31, 32, 33, respectively;
(6) the selected VL CDRs 1, 2, 3 amino acid sequences are set forth in SEQ ID NOs: 34, 35, 36, respectively;
(7) the selected VL CDRs 1, 2, 3 amino acid sequences are set forth in SEQ ID NOs: 108, 109, 110, respectively;
(8) the selected VL CDRs 1, 2, 3 amino acid sequences are set forth in SEQ ID NOs: 111, 112, 113, respectively;
(9) the selected VL CDRs 1, 2, 3 amino acid sequences are set forth in SEQ ID NOs: 114, 115, 116, respectively;
(10) the selected VL CDRs 1, 2, 3 amino acid sequences are set forth in SEQ ID NOs: 117, 118, 119, respectively;
(11) the selected VL CDRs 1, 2, 3 amino acid sequences are set forth in SEQ ID NOs: 120, 121, 122, respectively;
(12) the selected VL CDRs 1, 2, 3 amino acid sequences are set forth in SEQ ID NOs: 123, 124, 125, respectively;
(13) the selected VL CDRs 1, 2, 3 amino acid sequences are set forth in SEQ ID NOs: 126, 127, 128, respectively;
(14) the selected VL CDRs 1, 2, 3 amino acid sequences are set forth in SEQ ID NOs: 129, 130, 131, respectively;
(15) the selected VL CDRs 1, 2, 3 amino acid sequences are set forth in SEQ ID NOs: 132, 133, 134, respectively;
(16) the selected VL CDRs 1, 2, 3 amino acid sequences are set forth in SEQ ID NOs: 135, 136, 137, respectively;
(17) the selected VL CDRs 1, 2, 3 amino acid sequences are set forth in SEQ ID NOs: 138, 139, 140, respectively;
(18) the selected VL CDRs 1, 2, 3 amino acid sequences are set forth in SEQ ID NOs: 141, 142, 143, respectively;
(19) the selected VL CDRs 1, 2, 3 amino acid sequences are set forth in SEQ ID NOs: 180, 181, 182, respectively;
(20) the selected VL CDRs 1, 2, 3 amino acid sequences are set forth in SEQ ID NOs: 183, 184, 185, respectively;
(21) the selected VL CDRs 1, 2, 3 amino acid sequences are set forth in SEQ ID NOs: 186, 187, 188, respectively;
(22) the selected VL CDRs 1, 2, 3 amino acid sequences are set forth in SEQ ID NOs: 189, 190, 191, respectively;
(23) the selected VL CDRs 1, 2, 3 amino acid sequences are set forth in SEQ ID NOs: 192, 193, 194, respectively;
(24) the selected VL CDRs 1, 2, 3 amino acid sequences are set forth in SEQ ID NOs: 195, 196, 197, respectively;
(25) the selected VL CDRs 1, 2, 3 amino acid sequences are set forth in SEQ ID NOs: 198, 199, 200, respectively;
(26) the selected VL CDRs 1, 2, 3 amino acid sequences are set forth in SEQ ID NOs: 201, 202, 203, respectively;
(27) the selected VL CDRs 1, 2, 3 amino acid sequences are set forth in SEQ ID NOs: 204, 205, 206, respectively;
(28) the selected VL CDRs 1, 2, 3 amino acid sequences are set forth in SEQ ID NOs: 207, 208, 209, respectively;
(29) the selected VL CDRs 1, 2, 3 amino acid sequences are set forth in SEQ ID NOs: 210, 211, 212, respectively; and
(30) the selected VL CDRs 1, 2, 3 amino acid sequences are set forth in SEQ ID NOs: 213, 214, 215, respectively.

In one aspect, the disclosure is related to an antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDR 1 is SYVMH (SEQ ID NO: 99); the selected VH CDR2 is VISYX₁X₂SDKYYADSVKG (SEQ ID NO: 100); the selected VH CDR3 is GRGYRYGQSYYYGMDV (SEQ ID NO: 101);
(2) the selected VH CDR 1 is SYVVH (SEQ ID NO: 102); the selected VH CDR2 is VISYX₁X₂DNKFYADSVKG (SEQ ID NO: 103); the selected VH CDR3 is GRX₅YX₆X₇YYGLDV (SEQ ID NO: 104);
(3) the selected VH CDR 1 is TYVMH (SEQ ID NO: 105); the selected VH CDR2 is VISYX₁X₂NNKYYADSVKG (SEQ ID NO: 106); the selected VH CDR3 is GRSYVNYYGLDV (SEQ ID NO: 107);
(4) the selected VH CDR 1 is GFSFSSY (SEQ ID NO: 171); the selected VH CDR2 is SYX₁X₂SD (SEQ ID NO: 172); the selected VH CDR3 is GRGYRYGQSYYYGMDV (SEQ ID NO: 173);
(5) the selected VH CDR 1 is GFTFSSY (SEQ ID NO: 174); the selected VH CDR2 is SYX₁X₂DN (SEQ ID NO: 175); the selected VH CDR3 is GRX₅YX₆X₇YYGLDV (SEQ ID NO: 176); and
(6) the selected VH CDR 1 is GFTFSTY (SEQ ID NO: 177); the selected VH CDR2 is SYX₁X₂NN (SEQ ID NO: 178); the selected VH CDR3 is GRSYVNYYGLDV (SEQ ID NO: 179);
wherein the selected VL CDRs 1, 2, and 3 amino acid sequences are one of the following:
(1) the selected VL CDR 1 is KSSQSLLYSX₃X₄KTYLY (SEQ ID NO: 135); the selected VL CDR2 is EVSNRFS (SEQ ID NO: 136); the selected VL CDR3 is MQSIKLPLT (SEQ ID NO: 137);
(2) the selected VL CDR 1 is KSSQSLLYSX₃X₄KTYLY (SEQ ID NO: 138); the selected VL CDR2 is EVSNRFS (SEQ ID NO: 139); the selected VL CDR3 is MQSLKVPPT (SEQ ID NO: 140);
(3) the selected VL CDR 1 is KSSQSLLYSX₃X₄KTYLY (SEQ ID NO: 141); the selected VL CDR2 is EVSNRFS (SEQ ID NO: 142); the selected VL CDR3 is MQSVKIPLT (SEQ ID NO: 143);
(4) the selected VL CDR 1 is KSSQSLLYSX₃X₄KTYLY (SEQ ID NO: 207); the selected VL CDR2 is EVSNRFS (SEQ ID NO: 208); the selected VL CDR3 is MQSIKLPLT (SEQ ID NO: 209);
(5) the selected VL CDR 1 is KSSQSLLYSX₃X₄KTYLY (SEQ ID NO: 210); the selected VL CDR2 is EVSNRFS (SEQ ID NO: 211); the selected VL CDR3 is MQSLKVPPT (SEQ ID NO: 212); and
(6) the selected VL CDR 1 is KSSQSLLYSX₃X₄KTYLY (SEQ ID NO: 213); the selected VL CDR2 is EVSNRFS (SEQ ID NO: 214); the selected VL CDR3 is MQSVKIPLT (SEQ ID NO: 215);
wherein X₁ and X₃ in each sequence are independently selected from the group consisting of D, S, and A,
wherein X₂ and X₄ in each sequence are independently selected from the group consisting of G and A,
wherein X₅ in SEQ ID NO: 104 or 176 is independently selected from the group consisting of N and S,
wherein X₆ in SEQ ID NO: 104 or 176 is independently selected from the group consisting of Y and V,
wherein X₇ in SEQ ID NO: 104 or 176 is independently selected from the group consisting of Y and N.

In some embodiments, humanization may involve limited modifications on the CDRs. However, these modifications will have limited impact on the binding affinities. In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a light chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a light chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a light chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition. In some embodiments, the CDR is determined based on Chothia definition.

In some embodiments, there are no more than one insertions, deletions, or substitutions. In some embodiments, there are no more than two insertions, deletions, or substitutions. In some embodiments, there are no more than three insertions, deletions, or substitutions. In some embodiments, there are no more than four insertions, deletions, or substitutions. In some embodiments, the VH is at least 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOs: 37-39, 63-65, 69-71, 216, and 261-267. In some embodiments, the VL is at least 80%, 81%, 82%, 83%, 84%, 85%, 86%, 87%, 88%, 89%, 90%, 91%, 92%, 93%, 94%, 95%, 96%, 97%, 98%, 99% or 100% identical to any one of SEQ ID NOs: 40-42 and 66-68.

The disclosure also provides antibodies or antigen-binding fragments thereof that bind to CCR8. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 37, and the selected VL sequence is SEQ ID NO: 40. In some embodiments, the selected VH sequence is SEQ ID NO: 38 and the selected VL sequence is SEQ ID NO: 41. In some embodiments, the selected VH sequence is SEQ ID NO: 39 and the selected VL sequence is SEQ ID NO: 42. In some embodiments, the selected VH sequence is SEQ ID NO: 261 and the selected VL sequence is SEQ ID NO: 41. In some embodiments, the selected VH sequence is SEQ ID NO: 262 and the selected VL sequence is SEQ ID NO: 41. In some embodiments, the selected VH sequence is SEQ ID NO: 263 and the selected VL sequence is SEQ ID NO: 41. In some embodiments, the selected VH sequence is SEQ ID NO: 264 and the selected VL sequence is SEQ ID NO: 41. In some embodiments, the selected VH sequence is SEQ ID NO: 265 and the selected VL sequence is SEQ ID NO: 41. In some embodiments, the selected VH sequence is SEQ ID NO: 266 and the selected VL sequence is SEQ ID NO: 41. In some embodiments, the selected VH sequence is SEQ ID NO: 267 and the selected VL sequence is SEQ ID NO: 41.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIGS. 2A-2C or FIGS. 3A-3C or have sequences as shown in FIG. 4. When the polypeptides are paired with corresponding polypeptide (e.g., a corresponding heavy chain variable region or a corresponding light chain variable region), the paired polypeptides bind to CCR8 (e.g., human CCR8).

The anti-CCR8 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to CCR8 will retain an ability to bind to CCR8. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding. In some embodiments, the polypeptide comprises a linker with the sequence of GGGS (SEQ ID NO: 61) or GGGSGGGS (SEQ ID NO: 62).

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides various antibodies and antigen-binding fragments thereof derived from anti-CCR8 antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, V_{H}) and multiple constant domains (or constant regions), bind to one another via disulfide bonding within their constant domains to form the “stem” of the antibody. The light chains, which each contain one variable domain (or variable region, V_{L}) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E.A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, the antibodies or antigen-binding fragment thereof as described herein comprise heavy chain constant regions (e.g., CH, CH2, and/or CH3) selected from mouse IgG1, IgG2a, and IgG2b. In some embodiments, the antibodies or antigen-binding fragment thereof as described herein comprise light chain constant regions (e.g., CL) selected from mouse kappa or lambda CL.

In some embodiments, the antibodies or antigen-binding fragment thereof as described herein comprise heavy chain constant regions (e.g., CH1, CH2, and/or CH3) selected from human IgG (e.g., human IgG1, IgG2, IgG3, or IgG4). In some embodiments, the antibodies or antigen-binding fragment thereof as described herein comprise light chain constant regions (e.g., CL) selected from human kappa or lambda CL.

### Antibody Characteristics

The antibodies or antigen-binding fragments thereof described herein can block the binding between CCR8 and CCR8 ligands (e.g., CCL1). In some embodiments, by binding to CCR8, the antibody can inhibit CCL1-induced suppressive function of tumor resident Treg cells. In some embodiments, the antibody can upregulate immune response. In some embodiments, the antibody can deplete Treg cells (e.g., tumor-resident Treg cells). In some embodiments, the antibody has reduced risk of inducing autoimmunity and reduced risk of inducing systemic Treg cell depletion.

In some embodiments, the antibodies or antigen-binding fragments thereof as described herein increase antitumor immunity of at least or about 50%, 60%, 70%, 80%, 90%, 95%, 98%, or 100%.

In some embodiments, the antibodies or antigen-binding fragments thereof as described herein have less risk of inducing autoimmunity or causing systemic Treg cell depletion. In some embodiments, the antibodies or antigen-binding fragments thereof as described herein has a lower risk of inducing autoimmunity by least or about 10%, 20%, 30%, 40%, 50%, 60%, 70%, 80%, 90% as compared to other therapeutic agents that target Tregs (e.g., anti-CCR4, CTLA4, OX40, or GITR antibodies).

In some embodiments, the antibodies or antigen-binding fragments thereof as described herein can decrease activity or number of Treg cells (e.g., tumor-resident Treg cells and/or at the tumor microenvironment) by least or about 10%, 20%, 30%, 40%, 50%, 60%, 70%, 80%, or 90%, e.g., as compared to the activity or number of Treg cells when the antibodies or antigen-binding fragments thereof are not administered.

In some embodiments, the antibodies or antigen-binding fragments thereof as described herein can increase immune response, activity or number of immune cells (e.g., T cells, CD8+ T cells, CD4+ T cells, macrophages, antigen presenting cells) by at least or about 10%, 20%, 30%, 40%, 50%, 60%, 70%, 80%, 90%, 100%, 2 folds, 3 folds, 5 folds, 10 folds, or 20 folds, e.g., as compared to the case when the antibodies or antigen-binding fragments thereof are not administered.

In some embodiments, the antibodies or antigen-binding fragments thereof as described herein can effectively increase Teff: Treg ratio, e.g., by at least or about 10%, 20%, 30%, 40%, 50%, 60%, 70%, 80%, 90%, 100%, 2 folds, 3 folds, 5 folds, 10 folds, or 20 folds, e.g., as compared to the case when the antibodies or antigen-binding fragments thereof are not administered.

In some implementations, the antibody (or an antigen-binding fragment thereof) specifically binds to CCR8 (e.g., human CCR8, monkey CCR8 (e.g., cynomolgus monkey (Macaca fascicularis), mouse CCR8, and/or chimeric CCR8) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, or greater than 1×10⁻¹² M.

In some embodiments, the antibody or an antigen-binding fragment thereof binds to human CCR8 at Y17, F21, D97, W99, F101, G102, C106, G179, C183, W194, K195, 1196, and/or L278.

In some embodiments, the binding affinity of the antibodies or antigen-binding fragments thereof as described herein can be measured by FACS. An exemplary procedure is shown in Example 4. In some embodiments, EC50 is less than 25 μg/mL, less than 15 μg/mL, less than 10 μg/mL, less than 5 μg/mL, less than 2.5 μg/mL, less than 1 μg/mL, less than 0.5 μg/mL, less than 0.4 μg/mL, less than 0.3 μg/mL, less than 0.2 μg/mL, less than 0.15 μg/mL, less than 0.1 μg/mL, or less than 0.05 μg/mL. In some embodiments, EC50 is about 0.05-0.5 μg/mL, about 0.1-1 μg/mL, about 0.1-0.8 μg/mL, about 0.1-0.5 μg/mL, about 0.2-2 μg/mL, about 0.2-1 μg/mL, or about 0.3-5 μg/mL.

In some embodiments, the blockade of hCCL1 binding to hCCR8 by the antibodies or antigen-binding fragments thereof as described herein can be measured by Fluorescent Imaging Plate Reader (FLIPR) Calcium 6 Assay Kit. In some embodiments, EC50 is less than 30 nM, 20 nM, 10 nM, 5 nM, 2.5 nM, 1.5 nM, or 1 nM. In some embodiments, EC50 is about 1-10 nM, 1.5-20 nM, or 2.5-30 nM. In some embodiments, the maximum inhibition is greater than 10%, 20%, 30%, 40%, 50%, 60%, 70%, 80%, 90%, or 95%. In some embodiments, the maximum inhibition is less than 10%, 20%, 30%, 40%, 50%, 60%, 70%, 80%, 90%, or 95%.

In some embodiments, the antibodies can bind to one or more of the following human CCR2, human CCR4, human CCR5, human CCR8, and human CX3CR1. In some embodiments, the antibodies cannot bind to one or more of the following human CCR2, human CCR4, human CCR5, human CCR8, and human CX3CR1.

In some embodiments, the antibody-dependent cellular cytotoxicity (ADCC) can be determined by a reporter gene bioassay (ADCC reporter bioassay). In some embodiments, EC50 is less than 25 μg/mL, less than 15 μg/mL, less than 10 μg/mL, less than 5 μg/mL, less than 2.5 μg/mL, less than 1 μg/mL, less than 0.5 μg/mL, less than 0.4 μg/mL, less than 0.3 μg/mL, less than 0.2 μg/mL, less than 0.15 μg/mL, less than 0.1 μg/mL, less than 0.05 μg/mL, less than 0.025 μg/mL, less than 0.01 μg/mL, less than 0.005 μg/mL, or less than 0.0025 μg/mL. In some embodiments, EC50 is about 0.0025-0.05 μg/mL, about 0.005-0.1 μg/mL, about 0.005-0.05 μg/mL, about 0.005-0.01 μg/mL, about 0.01-0.2 μg/mL, or about 0.025-0.3 μg/mL.

General techniques for measuring the affinity of an antibody for an antigen include, e.g., flow cytometry, FACS, ELISA, RIA, and surface plasmon resonance (SPR). In some embodiments, the antibody binds to human CCR8 (SEQ ID NO: 43).

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95 °C.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen-binding fragments thereof as described herein block the binding of CCR8 to its ligand (e.g., CCL1). In some embodiments, the antibodies or antigen-binding fragments thereof as described herein inhibit CCL-1 induced suppressive function of tumor-resident Treg cells.

In some embodiments, the antibodies or antigen binding fragments can induce complement-dependent cytotoxicity (CMC) and/or antibody dependent cellular cytotoxicity (ADCC), and kill the cells that express CCR8 (e.g., Treg cells).

In some embodiments, the antibodies or antigen binding fragments have a functional Fc region. In some embodiments, effector function of a functional Fe region is antibody-dependent cell-mediated cytotoxicity (ADCC). In some embodiments, the antibodies or antigen binding fragments can induce complement mediated cytotoxicity (CMC).

In some embodiments, the Fe region is human IgG1, human IgG2, human IgG3, or human IgG4. In some embodiments, the antibody is a human IgG1 antibody.

In some embodiments, the antibodies or antigen binding fragments do not have a functional Fc region. For example, the antibodies or antigen binding fragments are Fab, Fab′, F(ab′)2, and Fv fragments. In some embodiments, the Fc region has LALA mutations (L234A and L235A mutations in EU numbering), or LALA-PG mutations (L234A, L235A, P329G mutations in EU numbering).

In some embodiments, the Fe region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments do not have a functional Fc region. For example, the antibodies or antigen binding fragments are Fab, Fab′, F(ab′)2, and Fv fragments. In some embodiments, the Fe region has LALA mutations (L234A and L235A mutations according to EU numbering), or LALA-PG mutations (L234A, L235A, P329G mutations according to EU numbering). In some embodiments, the Fe region has FLAA mutations (F234A and L235A according to EU numbering). In some embodiments, the Fc has SI mutations (S239D and 1332E mutations according to EU numbering). In some embodiments, the Fe has N297A mutation according to EU numbering. In some embodiments, the Fe has YTE mutations (M252Y, S254T and T256E according to EU numbering).

### Methods of Making Anti-CCR8 Antibodies

An isolated membrane protein of human CCR8 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of CCR8 and encompasses an epitope of the protein such that an antibody raised against the peptide forms a specific immune complex with the protein. As described above, the full length sequence of human CCR8 is known in the art (SEQ ID NO: 43).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human CCR8). The preparation can further include an adjuvant, such as Freund's complete or incomplete adjuvant, or a similar immunostimulatory agent.

Polyclonal antibodies can be prepared as described above by immunizing a suitable subject with a CCR8 polypeptide, or an antigenic peptide thereof (e.g., part of CCR8) as an immunogen. The antibody titer in the immunized subject can be monitored over time by standard techniques, such as with an enzyme-linked immunosorbent assay (ELISA) using the immobilized CCR8 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al. “Replacing the complementarity-determining regions in a human antibody with those from a mouse.” Nature 321.6069 (1986): 522; Riechmann et al. “Reshaping human antibodies for therapy.” Nature 332.6162 (1988): 323; Dall'Acqua et al. “Antibody humanization by framework shuffling.” Methods 36.1 (2005): 43-60; each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al. “A humanized CD18 antibody can block function without cell destruction.” The Journal of Immunology 151.4 (1993): 2296-2308; Chothia, et al., “Canonical structures for the hypervariable regions of immunoglobulins.” Journal of molecular biology 196.4 (1987): 901-917).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-CCR8 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chain. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant regions (e.g., IgG1, IgG2, IgG3, and IgG4).

Identity or homology with respect to an original sequence is usually the percentage of amino acid residues present within the candidate sequence that are identical with a sequence present within the human, humanized, or chimeric anti-CCR8 antibody or fragment, after aligning the sequences and introducing gaps, if necessary, to achieve the maximum percent sequence identity, and not considering any conservative substitutions as part of the sequence identity.

Additional modifications to the anti-CCR8 antibodies or antigen-binding fragments can be made. For example, a cysteine residue(s) can be introduced into the Fe region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. Wolff et al. (“Monoclonal antibody homodimers: enhanced antitumor activity in nude mice.” Cancer research 53.11 (1993): 2560-2565). Alternatively, an antibody can be engineered which has dual Fc regions.

In some embodiments, a covalent modification can be made to the anti-CCR8 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fe region. For example, the amount of fucose in such antibody composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fe region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods in Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, stomach cancer, testis cancer, gallbladder cancer, bile duct cancer, esophagus cancer, thymic carcinoma, acute myeloid leukemia, lymphoma lymphoblastic, transitional cell carcinoma or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the subject has a PD-1 inhibitor resistant cancer. In some embodiments, the subject is not responsive to an anti-PD-1 antibody or an anti-PD-L1 antibody treatment.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal.

A typical daily dosage of an effective amount of an antibody is 0.01 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, an anti-B7-13 antibody, an anti-CLDN18 antibody, an anti-SIGLEC15 antibody, an anti-41BB antibody, an anti-CD40 antibody, or an anti-GITR antibody.

In some embodiments, the additional therapeutic agent is a PD-I pathway inhibitor, e.g., an anti-PD-1 antibody or anti-PD-L1 antibody. Exemplary anti-PD-1 antibodies include e.g., Pembrolizumab, Nivolumab, Cemiplimab, Spartalizumab, Camrelizumab, Sintilimab, Tislelizumab, Toripalimab, and Dostarlimab. Exemplary anti-PD-L1 antibodies include e.g., Atezolizumab, Avelumab, Durvalumab, and KN035.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Anti-CCR8 Antibodies

To generate antibodies against human CCR8, RenMice™ (i.e., engineered mice comprising DNA encoding human immunoglobulin heavy and kappa light chain variable regions, for example, RenMab™ mice, RenLite™ mice) were immunized with human CCR8 protein. The antibody immune response was monitored by an antigen-specific immunoassay.

A total of 4 immunizations were performed. The immunizations were separated by two weeks. 5~6 days after the last immunization, retro-orbital blood was collected, and the antibody titer in serum was determined by Fluorescence-Activated Cell Sorting (FACS).

Spleen tissues were then collected and grinded. Spleen cells were first selected by CD3ε microbeads and anti-mouse IgM Microbeads, and then fused with SP2/0 cells (to generate hybridoma cells). The cells were then plated in 96-well plates with hypoxanthine-aminopterin-thymidine (HAT) medium.

Primary screening of the hybridoma supernatant in the 96-well plates was performed using FACS following standard procedures. Sub-cloning was performed using ClonePix® 2. In short, the positive wells identified during the primary screening were transferred to semisolid medium, and IgG positive clones were identified and tested. FITC anti-mouse IgG Fe antibody was used. Then, positive hybridoma cells were injected intraperitoneally to mice. Monoclonal antibodies were produced by growing hybridoma cells within the peritoneal cavity of the mice. The hybridoma cells multiplied and produced ascites fluid in the abdomens of the mice. The fluid contained a high concentration of antibodies which can be harvested for later use. Antibodies in ascites fluid were purified using GE AKTA™ protein chromatography (GE Healthcare, Chicago, Illinois, United States).

In another experiment, when a desired immune response was achieved, antigen-specific immune cells were isolated from the immunized mice to further obtain anti-CCR8 antibodies or to obtain the light chain and heavy chain variable region sequences of the anti-CCR8 antibodies. For example, single cell technology (for example, using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were used for antibody expression. The binding affinity to CCR8 was determined using FACS.

In another experiment, phage display was performed to screen and find monoclonal antibodies that are antigen-specific.

Exemplary antibodies obtained by this method included: 11B12, 11F8 and 11F5.

In another experiment, the “DG” in VH CDR2 of the antibodies described herein was mutated to “SG,” “AG” or “DA” to create CDR-modified VHs. For example, in one embodiment, the “DG” in VH CDR2 of 11B12 was mutated to “SG,” and the resulting CDR-modified VH is named 11B12-SG VH. The sequences of some of these CDR-modified VHs can be found in FIG. 4.

In another experiment, the “DG” in VL CDR1 of the antibodies described herein was mutated to “SG,” “AG,” or “DA,” to create CDR-modified VLs. For example, in one embodiment, the “DG” in VL CDR1 of 11B12 was mutated to SQ and the resulting CDR-modified VL is named 11B12-SG VL. The sequences of some of these CDR-modified VLs can be found in FIG. 4.

In another experiment, the “DNS” in the VH framework region of the antibodies described herein was mutated to “DNA,” to create FR-modified VHs. For example, in one example, the “DNS” in the framework of the VH of 11B12 was mutated to “DNA,” and the resulting FR-modified VH is named 11B12-DNA VH. The sequences of some of these FR-modified VHs can be found in FIG. 4 (e.g., SEQ ID NOs: 69-71). Thus, the antibody has a S74A mutation (Kabat numbering).

In another experiment, the VH was modified by both a FR modification (e.g., DNA) and a CDR modification (e.g., SG). In one example, the “DNS” in the framework of the VH of 11B12 is mutated to “DNA,” and the “DG” in VH CDR2 of 11B12 is mutated to “SG,” and the resulting FR-modified and CDR-modified VH is named 11B12-SG-DNA VH. The sequence of the resulting FR-modified and CDR-modified VH can be found in FIG. 4 (e.g., SEQ ID NO: 216).

In another experiment, the amino acid “N” at position 3 in VH CDR3 of the 11F5 was mutated to “S,” the amino acid “Y” at position 5 in VH CDR3 of the 11F5 was mutated to “V,” and/or the amino acid “Y” at position 6 in VH CDR3 of the 11F5 was mutated to “N” to create 11F5 CDR3-modified VHs. For example, the amino acid “N” at position 3 in VH CDR3 of the 11F5 was mutated to “S” and the resulting CDR-modified V is named 11F5-N3S VH. The amino acid “Y” at position 5 in VH CDR3 of the 11F5 was mutated to “V” and the resulting CDR-modified VH is named 11F5-Y5V VH. The amino acid “Y” at position 6 in VH CDR3 of the 11F5 was mutated to “N” and the resulting CDR-modified VH is named 11F5-Y6N VH. The amino acid “N” at position 3 and amino acid “Y” at position 5 in VH CDR3 of the 11F5 were mutated to “S” and “V,” respectively, and the resulting CDR-modified VH is named 11F5-N3S-Y5V VH. The amino acid “N” at position 3 and the amino acid “Y” at position 6 in VH CDR3 of the 11F5 were mutated to “S” and “N,” respectively, and the resulting CDR-modified VH is named 11F5-N3S-Y6N VH. The amino acid “Y” at position 5 and the amino acid “Y” at position 6 in VH CDR3 of the 11F5 were mutated to “V” and “N,” respectively, and the resulting CDR-modified VH is named 11F5-Y5V-Y6N VH. The amino acid “N” at position 3, amino acid “Y” at position 5 and the amino acid “Y” at position 6 in VH CDR3 of the 11F5 were mutated to “S,” “V” and “N,” respectively, and the resulting CDR-modified VH is named 11F5-N3S-Y5V-Y6N VH. The sequences of these CDR-modified VHs can be found in FIG. 4.

In another experiment, the VH of an antibody described herein was paired with the VL of another antibody described herein to form antibodies. For example, in one embodiment, the VH of 11B12 was paired with the VL of 11F8, and the resulting antibody is named 11B12-11F8. In one embodiment, the VH of 11B12-SG was paired with the VL of 11F8-SG, and the resulting antibody is named 11B12-SG-11F8-SG. In one embodiment, the VH of 11B12-SG was paired with the VL of 11B12-SG, and the resulting antibody is named 11B12-SG-11B12-SG. In one embodiment, the VH of 11B12-SG-DNA was paired with the VL of 1IFS, and the resulting antibody is named 11B12-SG-DNA-11F8. Some of the disclosed VH and VL combinations are listed in FIG. 5. The sequences of the heavy and light chain variable regions of these antibodies are shown in FIG. 4. FIG. 2A and FIG. 3A show the heavy and light chain CDR sequences of 11B12, 11F8, and 11F5 according to Kabat definition and Chothia definition, respectively.

Various IgG1, IgG2 and IgG4 antibodies were made. If the VH and VL of 11B12 are connected to IgG1 constant regions, the antibody is named as 11B12. If the VH and VL of 11B12 are connected to IgG4 constant regions, the antibody is named as 11B12-IgG4. The constant region can also include some mutations. For example, when SI mutations (EU numbering: S239D and 1332E mutations) are introduced into the Fc region of 11B12, the resulting antibody is named as 11B12-SI.

### Example 2. Cross-Reactivity of Anti-CCR8 Antibodies

CHO-S cells expressing human CCR8 (CHO-S-hCCR8), CHO-S cells expressing mouse CCR8 (CHO-S-mCCR8), HEK293T cells expressing human CCR8 (293T-hCCR8) and HEK293T cells expressing cynomolgus CCR8 (293T-cynCCR8) were plated in a 96-well plate at a density of 5×10⁴ cells/well. Diluted sample anti-CCR8 antibodies (1 μg/mL) were added to the 96-well plate, and incubated at 4 °C for 30 minutes. Then, the cells were incubated with the secondary antibody anti-hIgG-Fc-Alexa Flour® 647 (RL1-H) (Jackson ImmunoResearch Laboratories, Inc., Cat #: 109-606-170) at 4 °C in the dark for 15 minutes before flow cytometry analysis.

CHO-S-hCCR8 cells were obtained by transfecting CHO-S cells with a vector encoding human CCR8 (hCCR8, SEQ ID NO: 43). CHO-S-mCCR8 cells were obtained by transfecting CHO-S cells with a vector encoding mouse CCR8 (mCCR8, SEQ ID NO: 44). 293T-hCCR8 cells were obtained by transfecting HEK293T cells (ATCC, Cat #: CRL3216™) with a vector encoding human CCR8. 293T-cynCCR8 cells were obtained by transfecting HEK293T cells with a vector encoding a construct containing the amino acid sequence of cynomolgus (monkey) CCR8 (cynCCR8, SEQ ID NO: 45).

The test results are shown in the tables below (Table 1 and Table 2). All three anti-CCR8 antibodies can bind to hCCR8, cynCCR8 and mCCR8.

TABLE 1
Anti-CCR8 antibody cross-species binding analyses

| | | Percentage of | |
|---|---|---|---|
| Antibody | Cells | Positive Cells (%) | Evaluation |
| Negative | CHO-S-hCCR8 | 0.75 | No binding |
| Control | CHO-S-mCCR8 | 0.11 | No binding |
| | 293T-hCCR8 | 0 | No binding |
| | 293T-cynCCR8 | 0.08 | No binding |
| hIgG1 | CHO-S-hCCR8 | 0.70 | No binding |
| | CHO-S-mCCR8 | 0.60 | No binding |
| | 293T-hCCR8 | 0 | No binding |
| | 293T-cynCCR8 | 0.16 | No binding |
| 11F5 | CHO-S-hCCR8 | 64.7 | Binding |
| | CHO-S-mCCR8 | 24.2 | Binding |
| | 293T-hCCR8 | 100 | Binding |
| | 293T-cynCCR8 | 94.3 | Binding |
| 11F8 | CHO-S-hCCR8 | 67.7 | Binding |
| | CHO-S-mCCR8 | 93.9 | Binding |
| | 293T-hCCR8 | 100 | Binding |
| | 293T-cynCCR8 | 99.1 | Binding |

TABLE 2
Anti-CCR8 antibody cross-species binding analyses

| | | Percentage of | |
|---|---|---|---|
| Antibody | Cells | Positive Cells (%) | Evaluation |
| Negative | CHO-S-hCCR8 | 1.33 | No binding |
| Control | CHO-S-mCCR8 | 1.38 | No binding |
| | 293T-hCCR8 | 0 | No binding |
| | 293T-cynCCR8 | 0.19 | No binding |
| hIgG1 | CHO-S-hCCR8 | 1.61 | No binding |
| | CHO-S-mCCR8 | 2.59 | No binding |
| | 293T-hCCR8 | 0.31 | No binding |
| | 293T-cynCCR8 | 0.11 | No binding |
| 11B12 | CHO-S-hCCR8 | 76.3 | Binding |
| | CHO-S-mCCR8 | 77.7 | Binding |
| | 293T-hCCR8 | 100 | Binding |
| | 293T-cynCCR8 | 99.9 | Binding |

### Example 3. Cross Binding Reactivity of Anti-CCR Antibodies to CCR8 Family Proteins

In each experiment, the CHO cells were transfected to express human CCR8 (CHO-S-hCCR8), human CCR2 (hCCR2, SEQ ID NO: 46) (CHO-S-hCCR2), human CCR4 (hCCR4, SEQ ID NO: 47) (CHO-S-hCCR4), human CCR5 (hCCR5, SEQ ID NO: 48) (CHO-S-hCCR5), or human CX3CR1 (hCX3CR1, SEQ ID NO: 49) (CHO-S-hCX3CR1).

CHO-S-hCCR8, CHO-S-hCCR2, CHO-S-hCCR4, CHO-S-hCCR5 and CHO-S-hCX3CR1 were plated in a 96-well plate at a density of 5×10⁴ cells/well. Diluted sample anti-CCR1 antibodies (1 g/mL) were added to the 96-well plate, and incubated at 4 °C for 30 minutes. Then, the cells were incubated with the secondary antibody Anti-hIgG-Fc-Alexa Flour® 647 (RL1-H) (Jackson ImmunoResearch Laboratories, Inc., Cat #: 109-606-170) at 4 °C in the dark for 15 minutes before flow cytometry analysis.

The test results are shown in the table below. None of the three anti-CCR8 antibodies can bind to hCCR2, hCCR4, hCCR5 or hCX3CR1.

TABLE 3
Cross binding reactivity of CCR8 family proteins

| | CCR8 family | Percentage of | |
|---|---|---|---|
| Antibody | Proteins | Positive Cells (%) | Evaluation |
| 11B12 | hCCR8 | 76.3 | Binding |
| | hCCR2 | 2.53 | No binding |
| | hCCR4 | 0.76 | No binding |
| | hCCR5 | 6.58 | No binding |
| | hCX3CR1 | 3.49 | No binding |
| 11F5 | hCCR8 | 54.3 | Binding |
| | hCCR2 | 0.42 | No binding |
| | hCCR4 | 0.21 | No binding |
| | hCCR5 | 0.42 | No binding |
| | hCX3CR1 | 0.78 | No binding |
| 11F8 | hCCR8 | 58.9 | Binding |
| | hCCR2 | 1.13 | No binding |
| | hCCR4 | 2.34 | No binding |
| | hCCR5 | 1.15 | No binding |
| | hCX3CR1 | 1.64 | No binding |
| Plozalizuzumab | hCCR8 | 1.38 | No binding |
| analog | hCCR2 | 55.6 | Binding |
| | hCCR4 | 2.88 | No binding |
| | hCCR5 | 0.44 | No binding |
| | hCX3CR1 | 0.79 | No binding |
| Mogamulizumab | hCCR8 | 1.06 | No binding |
| analog-SI | hCCR2 | 1.06 | No binding |
| | hCCR4 | 31.5 | Binding |
| | hCCR5 | 0.13 | No binding |
| | hCX3CR1 | 0.60 | No binding |
| Leronlimab | hCCR8 | 1.01 | No binding |
| analog | hCCR2 | 0.80 | No binding |
| | hCCR4 | 1.54 | No binding |
| | hCCR5 | 54.5 | Binding |
| | hCX3CR1 | 0.38 | No binding |
| BII-00313-FC-LALA | hCCR8 | 2.26 | No binding |
| | hCCR2 | 2.39 | No binding |
| | hCCR4 | 1.91 | No binding |
| | hCCR5 | 1.77 | No binding |
| | hCX3CR1 | 79.6 | Binding |
| ISO (Human | hCCR8 | 1.23 | No binding |
| IgG1, kappa) | hCCR2 | 0.85 | No binding |
| | hCCR4 | 1.16 | No binding |
| | hCCR5 | 0.67 | No binding |
| | hCX3CR1 | 3.43 | No binding |

Plozalizuzumab (MLN1202) is a humanized immunoglobulin G1 (IgG1) anti-CCR2 monoclonal antibody developed by Millenium Pharmaceuticals (Takeda). The VH and VL sequences of Plozalizuzumab analog are shown as SEQ ID NO: 50 and SEQ ID NO: 51, respectively.

Mogamulizumab (KW-0761) is a humanized immunoglobulin G1 (IgG1) anti-CCR4 monoclonal antibody developed by Kyowa Kirin. The VH and VL sequences of Mogamulizumab analog-SI are shown as SEQ ID NO: 52 and SEQ ID NO: 53, respectively.

Leronlimab is a fully humanized IgG4 monoclonal antibody targeting CCR5, developed by CytoDyn. The VH and VL sequences of Leronlimab analog are shown as SEQ ID NO: 54 and SEQ ID NO: 55, respectively.

BII-00313 is a single heavy chain variable domain (VHH) antibody targeting human CX3C chemokine receptor 1 (CX3CR1) variants, developed by Boehringer-Ingelheim (VH SEQ ID NO: 56). BII-00313-FC-LALA was constructed by linking BII-00313 variable domain to IgG1 Fe with LALA mutations (EU numbering: L234A and L235A mutations).

### Example 4. Binding Affinity Verification of Anti-CCR8 Antibodies 11F5, 11F8, and

11B12 The binding activity of anti-CCR8 antibodies (11B12, 11F8, 11F5 and 7B16 analog) to 293T-hCCR8 cells (HEK293T cells expressing human CCR8) or 293T-cynCCR8 (HEK293T cells expressing cynomolgus CCR8) was verified by flow cytometry. The secondary antibody used in the experiment was: Alexa Fluor® 647 anti human IgG Fey (Jackson ImmunoResearch Laboratories, Inc., Cat #:109-606-170). EC50 was determined using serially diluted sample anti-CCR8 antibodies. The results are shown in the table below.

7B16 is a humanized immunoglobulin G1 (IgG1) anti-CCR8 monoclonal antibody developed by JOUNCE THERAPEUTICS (see WO2021163064A2). The VH and VL sequences of 7B16 analog are shown as SEQ ID NO: 59 and SEQ ID NO: 60, respectively.

TABLE 4
Affinity test results

EC50 (μg/mL)

| Antibody | 293T-hCCR8 | 293T-cynCCR8 |
|---|---|---|
| 11B12 | 0.2098 | 0.1035 |
| 11F8 | 0.0814 | 0.7098 |
| 11F5 | 0.2853 | >2 |

The results showed that all three anti-CCR8 antibodies showed binding affinity to human CCR8 and monkey CCR8. Among them, 11B12 bound to both monkey CCR8 and human CCR8 with similar affinities. 11F8 and 11F5 bound to human CCR8 with a greater affinity comparing to monkey CCR8. However, 7B16 analog showed binding affinity to human CCR8, but no binding affinity to monkey CCR8 (data not shown).

In another experiment, the binding activity of anti-CCR8 antibodies (11B12, 11F8, 11F5, 10A11 analog and 7B16 analog) to 293T-hCCR8 cells (HEK293T cells expressing Human CCR8) or 293T-mCCR8 (HEK293T cells expressing mouse CCR8) was verified by flow cytometry. The secondary antibody used in the experiment was: PE Goat α-Human IgG Fc (SouthernBiotech, Cat #: 2014-09). EC50 was determined using serially diluted sample anti-CCR8 antibodies. The results are shown in the table below.

TABLE 5
Affinity test results

EC50 (μg/mL)

| Antibody | 293T-hCCR8 | 293T-mCCR8 |
|---|---|---|
| 11B12 | 0.194 | 0.177 |
| 11F8 | 0.104 | 0.127 |
| 11F5 | 0.287 | 0.400 |

10A11 is a humanized immunoglobulin G1 (IgG1) anti-CCR8 monoclonal antibody developed by Shionogi (see WO2020138489A1). The VH and VL sequences of 10All analog are shown as SEQ ID NO: 57 and SEQ ID NO: 58, respectively.

The results showed that anti-CCR8 antibodies 11B12, 11F8 and 11F5 showed good binding affinity to human CCR8 and mouse CCR8. 10A11 analog and 7B16 analog showed binding affinity to human CCR8, but no binding affinity to mouse CCR8 (data not shown).

### Example 5. Blockade of Calcium Flux on hCCR8-Expressing Cells by Anti-hCCR8 mAbs

Blockade of hCCL1 binding to hCCR8 by human IgG1 anti-CCR8 antibodies and CDR-modified and FR-modified anti-CCR8 antibodies were tested by conducting calcium (Ca) flux assays using human CCR8-expressing 293T cells. Fluorescent Imaging Plate Reader (FLIPR) Calcium 6 Assay Kit (Molecular Devices, Cat #: R8190) was used. Cells were seeded the day prior to the experiment and incubated overnight at 37 °C, 5% CO₂. The cells were then loaded with FLIPR Calcium 6 dye (Molecular Devices) including probenecid (Invitrogen) and PowerLoad (Invitrogen), and incubated for 30 minutes at 37 °C. Different anti-CCR8 mAbs were added to the cells and further incubated at 37 °C for 30 minutes. After incubation, the Ca flux fluorescent signal induced by 1 μg/mL of recombinant human CCL1 (BioLegend, Cat #: 582706) was measured using SpectraMax® iD3 (Molecular Devices) for 1 minute. The results are showed in the table below.

TABLE 6
Blockade of calcium flux

| | | Max |
|---|---|---|
| | | inhibition |
| Antibody | IC50 (nM) | (%) |
| 10A11 analog | 1.27 | 68 |
| 11B12 | 2.49 | 91 |
| 11F5 | 3.98 | 85 |
| 11F8 | 2.03 | 99 |
| 11B12-11F8 | 3.78 | 93 |
| 11F5-SG-11F5-SG | 2.23 | 90 |
| 11F8-SG-11F8-SG | 0.77 | 95 |
| 11B12-SG-11F8 | 0.98 | 99 |
| 11B12-DNA-11B12 | 3.28 | 97 |

11B12, 11F8, 11B12-11F8, 11F8-SG-11F8-SG, 11F5-SG-11F5-SG, 11B12-SG-11F8 and 11B12-DNA-11B12 showed almost complete inhibition. However, the inhibition of 10A11 analog was partial even at 10 μg/mL.

### Example 6. Biophysical Analysis and Stability of Anti-CCR8 Antibodies

The biophysical properties and stability of three anti-CCR8 antibodies 11B12, 11F8 and 11F5 were evaluated.

Three anti-CCR8 antibodies 11B12, 11F8 and 11F5 were buffer exchanged into pH 6.0 (3 mg/mL histidine, 80 mg/mL sucrose, and 0.2 mg/mL Tween 80). The antibodies were kept in sealed Eppendorf tubes at 40±2 °C, 60%±5% RH (hereinafter referred to as 40 °C) and 4±3 °C (hereinafter referred to as 4 °C) for 14 days, and their thermal stability were evaluated.

Three anti-CCR8 antibodies 11B12, 11F8 and 11F5 were loaded into protein A column and eluted with a buffer (0.1 mol/L HAc) at pH 3.5. Half of the antibodies were added into 2 M Tris buffer to make the pH into 7.5 immediately. The remain half were kept at pH 3.5 for 6 hours and then adjusted the pH into 7.5. The diluted antibodies were kept in sealed Eppendorf tubes at pH 3.5±0.1, 25±2 °C (hereinafter referred to as pH 3.5) for 6 hours to test stability at low pH.

Three anti-CCR8 antibodies 11B12, 11F8 and 11F5 were buffer exchanged into pH 6.0 (3 mg/mL histidine, 80 mg/mL sucrose, and 0.2 mg/mL Tween® 80) and added NH₄HCO₃ stock to final of 0.94% NH₄HCO₃ Samples were kept in sealed Eppendorf tubes at 40±2 °C, 60%±5% RH for 6 hours (hereinafter referred to as NH₄HCO₃ 6h), or 24 hours (hereinafter referred to as NH₄HCO₃ 24h) to test stability.

Specifically, the following tests were performed: (1) detecting the purity of antibodies by Size-Exclusion High Performance Liquid Chromatography (SEC-HPLC) (indicated as the percentage of the main peak area to the sum of all peak areas (Purity, %)); (2) detecting the apparent hydrophobicity of the antibodies using the Hydrophobic Interaction Chromatography-High Performance Liquid Chromatography (HIC-HPLC) method (indicated as the retention time of the main peak (HIC, min)); (3) detecting the pI (isoelectric point) and charge variants of the antibodies by the Capillary Isoelectric Focusing (cIEF) method (indicated as the percentages of the main component, acidic component, and alkaline component); (4) detecting the purity changes of antibodies by capillary electrophoresis-sodium dodecyl sulphate (CE-SDS) under non-reducing (CE-SDS(NR)) conditions (indicated as the percentage of the main peak area to the sum of all peak areas (Purity, %)); (5) detecting thermodynamic stability of the antibodies by the high throughput multifunctional protein stability analyzer (UNcle, Unchained Labs) (indicated as the melting temperature (Tm) and aggregation temperature (Tagg)).

In the SEC-HPLC experiments, the antibody samples were diluted to 1 mg/mL with purified water and an Agilent 1290 chromatograph system (connected with XBridge Protein BEH SEC column (200 Å, Waters Corporation)) was used. The following parameters were used: mobile phase: 0.1M phosphate buffer (PB)+10% ACN, pH 7.4; flow rate: 1.8 mL/min; column temperature: 25 °C; detection wavelength: 280 nm, 220 nm; injection volume: 10 μL; sample tray temperature: about 4 °C; and running time: 7 minutes.

In the HIC-HPLC experiments, an Agilent 1260 chromatograph system (connected with ProPac HIC-10 column (4.6×250 mm, Thermo Scientific)) was used, and samples were 10 times diluted using mobile phase A. The following parameters were used: mobile phase A: 0.9 M ammonium sulfate, 0.1 M phosphate buffer (PB), 10% acetonitrile pH 6.5; mobile phase B: 0.1 M phosphate buffer (PB), 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30 °C; detection wavelength: 280 nm, 220 nm; injection volume: 10 μg; sample tray temperature: about 10 °C; and running time: 50 minutes.

In the cIEF experiments, a Maurice™ cIEF Method Development Kit (ProteinSimple, Cat #: PS-MDK01-C) was used for sample preparation. Specifically, 8 μL, 30 μg protein sample was mixed with the following reagents in the kit: 1 μL Maurice™ cIEF pI Marker-7.05, 1 μL Maurice™ cIEF pI Marker-10.10, 35 μL 1% Methyl Cellulose Solution, 2 μL Maurice™ cIEF 500 mM Arginine, 1.33VL Ampholytes (Pharmalyte pH ranges 3-10), and 6.66 μL Ampholytes (Pharmalyte pH ranges 8-10.5) water (added to make a final volume of 100 μL). On the Maurice™ analyzer (ProteinSimple, Santa Clara, CA), Maurice™ cIEF Cartridges (PS-MC02-C) were used to generate imaging capillary isoelectric focusing spectra. The sample was focused for a total of 10 minutes.

In the CE-SDS experiments, Maurice™ (ProteinSimple, Maurice) and Maurice™ CE-SDS Size Application Kit (ProteinSimple, Cat #: PS-MAK02-S) were used.

In CE-SDS(NR), 30 μL Sample Buffer, 30 μL, 30 μg antibody sample, 1.5 μL 25× internal standard, 3 μL 250 nM Iodoacetamide (SIGMA, Cat #: 16125) were add to a microcentrifuge tube, followed by centrifugation at 3000 rpm for 1 minute and heating in a 70 °C water bath for 10 minutes. The samples were then cooled to room temperature followed by centrifugation at 10000 rpm for 3 minutes. Supernatant sample preparations were then transferred to a 96-well plate and tested in Maurice™. The following parameters were used: injection voltage 4.6 kV, injection time 20 see, separation voltage 5.75 kV, and separation time 40 min.

In the UNcle experiments, 8 μL protein sample was loaded into the Uni tube, and the system operated at a thermal temperature from 25 °C to 95 °C at a heating rate of 1 °C/min. The particle size and polydispersity were detected by dynamic light scattering (DLS) before heating. The protein stability was characterized by differential scanning full spectrum fluorescence (DSF), static light scattering (SLS) and dynamic light scattering (DLS).

Detailed results of 11B12, 11F8 and 11F5 are shown in the tables below. The results showed that 11B12, 11F8 and 11F5 exhibited good stability and biophysical properties.

TABLE 7
Biophysical properties test results

| | | | | UNcle | | |
|---|---|---|---|---|---|---|
| | SEC | HIC RT | | Tm2 | Tm3 | Tagg |
| Antibody | Purity (%) | (min) | pI/cIEF | (°C) | (°C) | (°C) |
| 11B12 | 100.00 | 8.685 | 9.268 | 71.50 | 89.09 | 66.85 |
| 11F5 | 97.11 | 11.703/14.169 | 8.765 | 71.31 | 87.12 | 63.21 |
| 11F8 | 100.00 | 6.589 | 9.2-9.4 | 70.99 | 87.3 | 50.00 |

TABLE 8
stability of anti-CCR8 antibodies

| | | SEC | | CE-SDS (NR) | | | cIEF | | | |
|---|---|---|---|---|---|---|---|---|---|---|
| | | Purity | HIC | Purity | HHL | HL | Acidic | | Alkali | |
| Antibody | Treatment | (%) | (min) | (%) | (%) | (%) | (%) | Main (%) | (%) | pI |
| 11B12 | 0 d | 100 | 9.61 | 92.4 | 5.8 | 0.3 | 30.6 | 67.9 | 1.6 | 9.173 |
| | 4 °C 14 d | 100 | 9.61 | 94.1 | 4.1 | 0.3 | 30.6 | 68.3 | 1.2 | 9.194 |
| | 40 °C 14 d | 98.74 | 9.61 | 91.4 | 5.8 | 0.4 | 39.8 | 58.9 | 1.3 | 9.190 |
| | pH 3.5 0 h | 100 | 9.60 | 94.5 | 4.0 | 0.3 | 31.0 | 67.4 | 1.6 | 9.195 |
| | pH 3.5 6 h | 100 | 9.60 | 93.0 | 5.6 | 0.3 | 33.9 | 64.5 | 1.6 | 9.205 |
| | NH₄HCO₃ 6 h | 98.62 | 9.59 | 93.5 | 4.2 | 0.3 | 31.3 | 67.1 | 1.6 | 9.197 |
| | NH₄HCO₃ 24 h | 98.64 | 9.60 | 93.3 | 4.7 | 0.3 | 31.1 | 67.3 | 1.6 | 9.204 |
| 11F5 | 0 d | 98.22 | 16.65 | 93.1 | 3.6 | 0.3 | 31.8 | 40.9 | 27.2 | 8.824 |
| | 4 °C 14 d | 98.44 | 16.68 | 93.1 | 3.7 | 0.3 | 30.3 | 42.0 | 27.7 | 8.793 |
| | 40 °C 14 d | 98.42 | 14.94 | 90.3 | 5.1 | 0.4 | 15.0 | 52.3 | 32.8 | 8.760 |
| | pH 3.5 0 h | 98.44 | 16.67 | 97.5 | 0.1 | 0.7 | 33.0 | 41.2 | 25.8 | 8.895 |
| | pH 3.5 6 h | 98.60 | 16.67 | 94.1 | 3.8 | 0.3 | 31.4 | 41.7 | 26.9 | 8.887 |
| | NH₄HCO₃ 6 h | 98.36 | 16.64 | 91.3 | 5.6 | 0.4 | 31.9 | 41.1 | 27.0 | 8.844 |
| | NH₄HCO₃ 24 h | 98.46 | 16.65 | 93.4 | 3.8 | 0.4 | 31.9 | 41.2 | 26.9 | 8.823 |
| 11F8 | 0 d | 100 | 8.1 | 91.7 | 4.9 | 0.5 | 52.2 | 47.8 | — | 9.555 |
| | 4 °C 14 d | 98.71 | 8.1 | 92.2 | 5.8 | 0.8 | 53.5 | 46.5 | — | 9.547 |
| | 40 °C 14 d | 95.35 | 8.2 | 86.2 | 8.1 | 3.4 | 73.9 | 26.1 | — | 9.567 |
| | pH 3.5 0 h | 98.23 | 8.2 | 95.1 | 4.3 | — | 62.8 | 37.3 | — | 9.557 |
| | pH 3.5 6 h | 98.67 | 8.1 | 96.4 | 3.2 | — | 50.9 | 49.1 | — | 9.561 |
| | NH₄HCO₃ 6 h | 98.73 | 8.1 | 91.1 | 6.9 | 0.8 | 59.6 | 40.4 | — | 9.552 |
| | NH₄HCO₃ 24 h | 98.67 | 8.1 | 91.1 | 6.5 | 0.7 | 56.1 | 43.9 | — | 9.544 |

### Example 7. Antibody-Dependent Cellular Cytotoxicity (ADCC) Determination

In a reporter gene bioassay (ADCC reporter bioassay), engineered Jurkat cells were used as effector cells (Jurkat-Luc-hCD16A-158V), and H293T cells with hCCR8 surface antigen were used as target cells. The cells were incubated with each antibody at a ratio of 4:1 for 6 hours to determine ADCC activity. The results are shown in the table below.

TABLE 9
ADCC determination

| Antibody | EC50 (μg/mL) |
|---|---|
| 7B16 analog-SI | 0.0038 |
| 11B12-SI | 0.0016 |
| 11F5-SI | 0.0039 |
| 11F8-SI | 0.0059 |
| 7B16 analog | 0.0178 |
| 11B12 | 0.0130 |
| 11F5 | 0.0084 |
| 11F8 | 0.0191 |

The results showed that all anti-CCR8 antibodies had an ADCC activity that was comparable to the positive control drug (71B16 analog), and SI mutation resulted in stronger ADCC effect compared to WT.

In another experiment, the ADCC activity of human IgG1 anti-CCR8 antibodies and CDR-modified and FR-modified anti-CCR8 antibodies were determined. The results are shown in the table below.

The results showed that anti-CCR8 antibodies 11B12-11F8, 11F5-SG-11F5-SG, 11F8-SG-11F8-SG, 11B12-SG-11F8 and 11B12-DNA-11B12 had an ADCC activity. Among them, 11B12-11F8, 11F5-SG-11F5-SG, 11F8-SG-11F8-SG and 11B12-SG-11F8 exhibited a better ADCC activity as compared to 11B12, 11F5 and 11F8.

TABLE 10
ADCC determination

| Antibody | EC50 (μg/mL) |
|---|---|
| 11B12-11F8 | 0.0075 |
| 11F5-SG-11F5-SG | 0.0055 |
| 11F8-SG-11F8-SG | 0.0044 |
| 11B12-SG-11F8 | 0.0075 |
| 11B12-DNA-11B12 | 0.0162 |

### Example 8. Anti-Tumor Activity of the Anti-CCR8 Antibodies

Anti-CCR8 antibodies were tested for their effects on tumor growth in vivo in a colon cancer model. About 5×10⁵ MC38 cells (murine colon cancer cells) were injected subcutaneously in each B-hCCR8 mouse (Biocytogen Pharmaceuticals (Beijing) Co., Ltd., Beijing, China; Cat #: 110096). When the tumors in the mice reached a volume of about 100 mm³, the mice were randomly placed into different groups based on tumor volumes. The mice were then injected with phosphate buffer saline (PBS) or anti-human CCR8 antibodies by intraperitoneal (i.p.) administration.

The injected volume was calculated based on the weight of the mouse and desired dosage of 10 mg/kg. The length of the long axis and the short axis of the tumor were measured and the volume of the tumor was calculated as 0.5× (long axis)×(short axis)².

The tumor growth inhibition percentage (TGI %) was calculated using the following formula: TGI (%)=[1−(Ti−T0)/(Vi−V0)]×100. Ti is the average tumor volume in the treatment group on day i. TO is the average tumor volume in the treatment group on day zero. Vi is the average tumor volume in the control group on day i. V0 is the average tumor volume in the control group on day zero.

Values are expressed as mean±SEM. T-test was performed for statistical analysis. A TGI % higher than 60% indicates clear suppression of tumor growth. P<0.05 is a threshold to indicate significant difference.

TABLE 11
Group assignment

| | No. | | | | | Total No. |
|---|---|---|---|---|---|---|
| Group | of mice | Antibodies | Dosage | Route | Frequency | of administration |
| G1 | 6 | PBS | — | i.p. | BIW | 6 |
| G2 | 6 | 10A11 analog | 10 mg/kg | i.p. | BIW | 6 |
| G3 | 6 | 7B16 analog | 10 mg/kg | i.p. | BIW | 6 |
| G4 | 6 | 11F5 | 10 mg/kg | i.p. | BIW | 6 |

The weight of mice in different groups all increased. On the day of grouping (Day 0), the average weight of each group was in the range of 19.8 g-20.3 g. At the end of the experiment (Day 21), the average weight of each group was in the range of 21.6 g-24.6 g, the average weight of each group was in the range of 111.4%-121.8%. The results showed that the tested antibodies were well tolerated and were not obviously toxic to the mice.

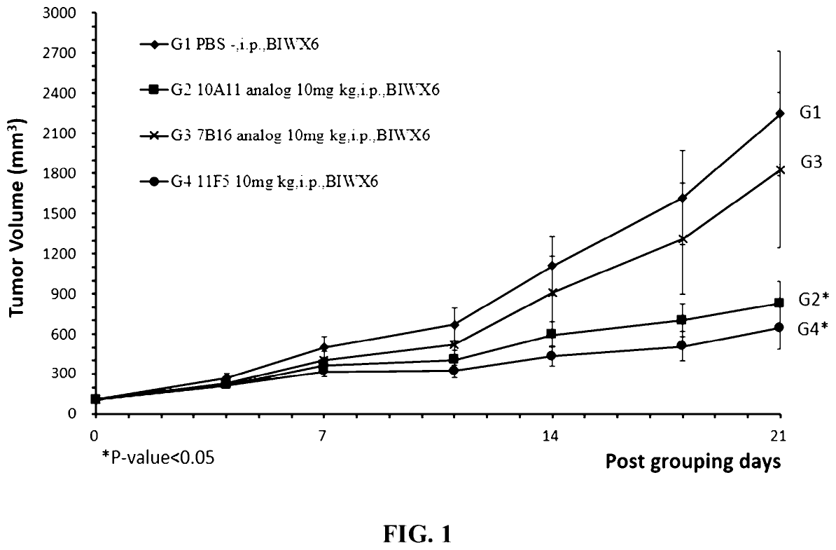

The tumor size in groups treated with the anti-CCR8 antibodies are shown in FIG. 1. The table below summarizes the results for this experiment, including the tumor volumes on the day of grouping (Day 0), 7 days after the grouping (Day 7), 14 days after the grouping (Day 14), and at the end of the experiment (Day 21); the survival rate of the mice; Tumor Growth Inhibition value (TGI); and the statistical differences (P value) of tumor volume and body weight between the treatment and control groups.

The results showed that 11F5 exhibited a better tumor growth inhibition effect than positive control 10A11 analog and 7B16 analog.

TABLE 12
Tumor size changes

| | | | | | | | | P value | |
|---|---|---|---|---|---|---|---|---|---|
| | | Tumor volume (mm³) | | | | | | Body | Tumor |
| Group | | Day 0 | Day 7 | Day 14 | Day 21 | Survival | TGI % | weight | Volume |
| Control | G1 | 109 ± 4 | 502 ± 80 | 1109 ± 221 | 2246 ± 465 | 6/6 | NA | NA | NA |
| Treat | G2 | 109 ± 5 | 365 ± 51 | 596 ± 97 | 830 ± 161 | 6/6 | 66.3 | 0.166 | 0.016 |
| | G3 | 108 ± 6 | 402 ± 71 | 912 ± 272 | 1828 ± 581 | 6/6 | 19.5 | 0.001 | 0.587 |
| | G4 | 109 ± 6 | 320 ± 42 | 435 ± 75 | 647 ± 163 | 6/6 | 74.8 | 0.007 | 0.015 |

In other similar experiments, anti-CCR8 antibodies 11F5, 11F5-SI and/or 11F8-SI were tested for their effects on tumor growth in vivo in the colon cancer model at a dosage of 3 mg/kg.

Table 13 and Table 14 summarize the tumor volumes on the day of grouping (Day 0), 14 days after the grouping (Day 14), and 21 days after the grouping (Day 21); Tumor Growth Inhibition value (TGI); and the statistical differences (P value) of tumor volume and body weight between the treatment and control groups in two different experiments.

TABLE 13
Tumor size changes

| | | | | | | P value | |
|---|---|---|---|---|---|---|---|
| | | Tumor volume (mm³) | | | | Body | Tumor |
| Group | Antibodies | Day 0 | Day 14 | Day 21 | TGI % | weight | Volume |
| G1 | PBS | 109 ± 3 | 903 ± 87 | 1993 ± 184 | NA | NA | NA |
| G2 | 11F5 | 109 ± 4 | 641 ± 44 | 1361 ± 163 | 33.5 | 0.649 | 0.028 |
| G3 | 11F5-SI | 109 ± 3 | 702 ± 175 | 1137 ± 323 | 45.4 | 0.507 | 0.044 |

TABLE 14
Tumor size changes

| | | | | | | P value | |
|---|---|---|---|---|---|---|---|
| | | Tumor volume (mm³) | | | | Body | Tumor |
| Group | Antibodies | Day 0 | Day 14 | Day 21 | TGI % | weight | Volume |
| G1 | PBS | 114 ± 6 | 793 ± 131 | 1516 ± 299 | NA | NA | NA |
| G2 | 11F5-SI | 113 ± 6 | 498 ± 90 | 808 ± 209 | 50.5 | 0.557 | 0.081 |
| G3 | 11F8-SI | 113 ± 6 | 605 ± 160 | 969 ± 290 | 39.0 | 0.452 | 0.218 |

The results showed that 11F5, 11F5-SI and 11F8-SI exhibited good tumor growth inhibition effects in colon cancer and 1 1F5-SI exhibited a better tumor growth inhibition effect than 11F5.

Further, in another experiment, 11F8-SI and 11B12-SI were also tested for there effects on tumor growth in a colon cancer model. About 5×10⁵ MC38 cells were injected subcutaneously in B-hPD-1/hPD-L1/hCCR8 mice (Biocytogen Pharmaceuticals (Beijing) Co., Ltd., Beijing, China; Cat #: 131764). When the tumors in the mice reached a volume of about 100-150 mm³, the mice were randomly placed into different groups based on tumor volumes (6 mice in each group). The mice were then injected with PBS (G1, control), 3 mg/kg 11F8-SI (G2), and 3 mg/kg 11B12-SI (G3) by intraperitoneal injection. The antibody was administered on the first day and the fourth day of each week for 3 weeks (6 injections in total).

The results showed that 11F5-SI and 11B12-SI exhibited good tumor growth inhibition effects with a TGI % of 44.34% and 37.60%, respectively.

### Example 9. Binding Affinity Verification of Anti-CCR8 Antibodies

The binding activity of anti-CCR8 antibodies to 293T-hCCR8 cells (obtained by transfecting HEK293T cells to express human CCR8) and 293T-mCCR8 cells (HEK293T cells expressing mouse CCR8) was verified by flow cytometry. The secondary antibody used in the experiment was: PE Goat α-Human IgG Fc (SouthernBiotech, Cat #: 2014-09). EC50 was determined using serially diluted sample anti-CCR8 antibodies. The results are shown in the table below.

TABLE 15
Affinity test results

EC50 (μg/mL)

| Antibody | 293T-hCCR8 | 293T-mCCR8 |
|---|---|---|
| 10A11 analog | 0.058 | >10 |
| 7B16 analog | 0.039 | >10 |
| 11B12 | 0.194 | 0.177 |
| 11F5 | 0.287 | 0.400 |
| 11F8 | 0.104 | 0.127 |
| 11B12-11F8 | 0.152 | 0.100 |
| 11F5-SG-11F5-SG | 0.033 | 0.069 |
| 11F8-SG-11F8-SG | 0.015 | 0.053 |
| 11B12-SG-11F8 | 0.051 | 0.055 |
| 11B12-DNA-11B12 | 0.144 | 0.084 |

In other experiments, the binding activities of CDR-modified and FR-modified anti-CCR8 antibodies to 293T-hCCR8 cells and 293T-mCCR8 were verified by flow cytometry. The results are summarized in the table below.

TABLE 16
Affinity test results

EC50 (μg/mL)

| Antibody | 293T-hCCR8 | 293T-mCCR8 |
|---|---|---|
| 11B12-11F5 | 0.298 | 0.171 |
| 11F8-11B12 | 0.075 | 0.155 |
| 11F8-11F5 | 0.137 | 0.116 |
| 11F5-11B12 | 0.132 | 0.163 |
| 11F5-11F8 | 0.162 | 0.184 |
| 11F5-SG-11F5-AG | 0.069 | 0.092 |
| 11F5-SG-11F5-DA | 0.150 | 0.138 |
| 11F5-DA-11F5-SG | 0.124 | — |
| 11F5-DA-11F5-AG | — | 0.463 |
| 11F5-DA-11F5-DA | 0.392 | 0.253 |
| 11F8-SG-11F8-AG | 0.050 | 0.218 |
| 11F8-SG-11F8-DA | 0.049 | 0.173 |
| 11F8-AG-11F8-SG | — | 0.149 |
| 11F8-AG-11F8-AG | 0.033 | 0.148 |
| 11F8-AG-11F8-DA | 0.120 | 0.171 |
| 11F8-DA-11F8-SG | 0.154 | — |
| 11F8-DA-11F8-AG | 0.076 | — |
| 11F8-DA-11F8-DA | 0.222 | 0.128 |
| 11B12-SG-11B12-DA | 0.064 | 0.059 |
| 11B12-AG-11B12-DA | 0.073 | 0.074 |
| 11B12-SG-11B12-SG | 0.048 | 0.041 |
| 11B12-AG-11B12-SG | 0.043 | 0.038 |
| 11B12-SG-11B12-AG | 0.047 | 0.042 |
| 11B12-11F8-SG | 0.165 | 0.120 |
| 11B12-SG-11F8-SG | 0.009 | 0.099 |

(“—” means no EC50 was calculated)

### Example 10. Epitope Analysis of Anti-hCCR8 Antibodies

To analyze the amino acid residues involved in the binding activity of the anti-hCCR8 antibodies to human CCR8, human CCR8 point mutants of amino acids in the extracellular domains (N-terminus, ECL1, ECL2, and ECL3) were produced, then the binding activity of 11F5, 11F8, 11F8-SG-11F8-SG, 11B12-11F8 and 11B12-SG-11F8 to the mutants was evaluated by flow cytometry. The analysis revealed the involvement of multiple amino acids of each extracellular domain (N-terminus, ECL1, ECL2, and ECL3) in the binding activity of the clones. In particular, a drastic reduction in binding activity of the clones was observed in the mutants in which Y17A (Y at position 17 of human CCR8 (SEQ ID NO: 43) is substituted for A, the same shall apply hereunder), F21A, D97A, W99A, F101A, G102A, C106A, G179A, C183A, W194A, K195A, I196A, and L278A. F at position 21 is conserved between human and mouse CCR8. Binding to F21 may be important for demonstrating cross-reactivity to mouse CCR8. The F21A mutant did not alter the binding activity of 10A11 analog and 7B16 analog. This may explain why these clones do not cross-react with mouse CCR8.

### Example 11. Combination Therapy with an Anti-PD-1 Antibody

To evaluate the efficacy of combination therapy, anti-CCR8 antibodies were administered to mice with another therapeutic agent Pembrolizumab analog.

Pembrolizumab analog is a humanized immunoglobulin G1 (IgG1) anti-PD-1 monoclonal antibody. The VH and VL sequences of Pembrolizumab analog are shown as SEQ ID NO: 217 and SEQ ID NO: 218, respectively.

About 5×10⁵ MC38 cells (murine colon cancer cells) were injected subcutaneously in B-hPD-1/hPD-L1/hCCR8 mice (Biocytogen Pharmaceuticals (Beijing) Co., Ltd., Beijing, China; Cat #: 131764). When the tumors in the mice reached a volume of about 100-150 mm³, the mice were randomly placed into different groups based on tumor volumes (6 mice in each group). The mice were then injected with PBS (G1, control), 0.3 mg/kg Pembrolizumab analog (G2), 3 mg/kg 11F5 (G3), or a combination of 0.3 mg/kg Pembrolizumab analog and 3 mg/kg 11F5 (G4) by intraperitoneal injection. The antibody was administered on the first day and the fourth day of each week for 3 weeks (6 injections in total).

The weight of the mice was monitored during the entire treatment period. The weight of mice in different groups all increased. On the day of grouping (Day 0), the average body weight of each group was in the range of 18.2 g-19.2 g. At the end of the experiment (Day 28), the average body weight of each group was in the range of 21.7 g-23.5 g, and the average body weight of each group was in the range of 114.4%-123.3%. The results showed that the tested antibodies were well tolerated and were not obviously toxic to the mice.

The table below summarizes the results for this experiment, including the tumor volumes on the day of grouping (Day 0), 14 days after the grouping (Day 14) and at the end of the experiment (Day 28); the survival rate of the mice; Tumor Growth Inhibition value (TGI); and the statistical differences (P value) of tumor volume and body weight between the treatment and control groups.

TABLE 17
Tumor size changes

| | | | | | | | P value | |
|---|---|---|---|---|---|---|---|---|
| | | Tumor volume (mm³) | | | | | Body | Tumor |
| Group | | Day 0 | Day 14 | Day 28 | Survival | TGI % | weight | Volume |
| Control | G1 | 99 ± 5 | 377 ± 28 | 1501 ± 186 | 6/6 | NA | NA | NA |
| Treat | G2 | 100 ± 6 | 359 ± 53 | 1040 ± 208 | 6/6 | 32.9 | 0.428 | 0.129 |
| | G3 | 99 ± 6 | 457 ± 79 | 1089 ± 330 | 6/6 | 29.4 | 0.182 | 0.302 |
| | G4 | 100 ± 5 | 322 ± 67 | 669 ± 160 | 6/6 | 59.4 | 0.005 | 0.007 |

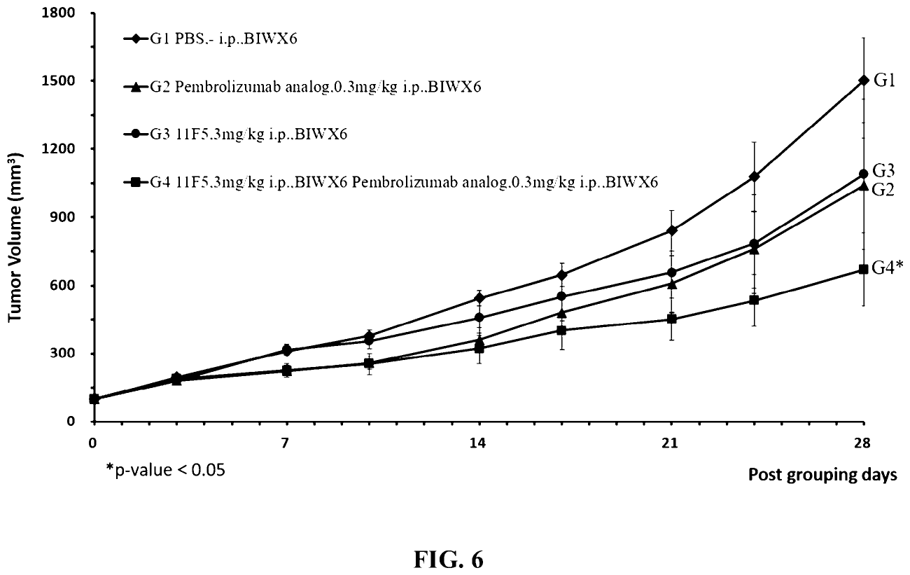

The tumor size for each treatment group is also shown in FIG. 6. The results showed that, as compared to the control group (G1), the treatment group (G2-G4) exhibited different anti-tumor effects. In addition, that combination of 11F5 and Pembrolizumab analog (G4) inhibited tumor growth with a higher TGI % than that of 11F5 (G3) or Pembrolizumab analog (G2).

In another similar experiment, the combinations of 11F5-SI and Pembrolizumab analog, or 11B12-SI and Pembrolizumab analog were tested for their effects on tumor growth in vivo in the colon cancer model to evaluate the efficacy of combination therapy. Details are shown in the table below.

TABLE 18
Group assignment

| | No. of | | | | | Total No. of |
|---|---|---|---|---|---|---|
| Group | mice | Antibodies | Dosage | Route | Frequency | administration |
| G1 | 6 | PBS | — | i.p. | BIW | 6 |
| G2 | 6 | Pembrolizumab analog | 0.3 mg/kg | i.p. | BIW | 6 |
| G3 | 6 | 11F5-SI | 3 mg/kg | i.p. | BIW | 6 |
| G4 | 6 | 11F5-SI | 3 mg/kg | i.p. | BIW | 6 |
| | | Pembrolizumab analog | 0.3 mg/kg | | | |
| G5 | 6 | 11B12-SI | 3 mg/kg | i.p. | BIW | 6 |
| G6 | 6 | 11B12-SI | 3 mg/kg | i.p. | BIW | 6 |
| | | Pembrolizumab analog | 0.3 mg/kg | | | |

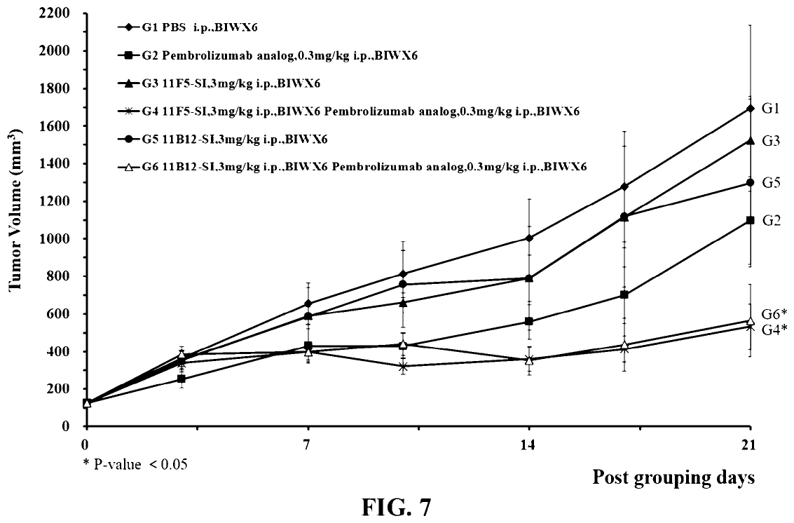

The tumor size for each treatment group is shown in FIG. 7. The results showed that the combination of 11F5-SI and Pembrolizumab analog (G4) inhibited tumor growth with a significantly higher TGI % (77.49%) than that of 11F5-SI (G3) or Pembrolizumab analog (G2). Similarly, the combination of 11B12-SI and Pembrolizumab analog (G6) inhibited tumor growth with a significantly higher TGI % (74.52%) than that of 11B12-SI (G5) or Pembrolizumab analog (G2).

### Example 12. Binding Affinity Verification of 11F5 and its CDR3-Modified Antibodies

The binding activities of 11F5 and its CDR3-modified antibodies to 293T-hCCR8 cells and 293T-mCCR8 cells were verified by flow cytometry. The secondary antibody used in the experiment was: PE Goat α-Human IgG Fc (SouthernBiotech, Cat #: 2014-09). EC50 was determined using serially diluted sample antibodies. The results are shown in the table below.

TABLE 19
Affinity test results

EC50 (μg/mL)

| Antibody | 293T-hCCR8 | 293T-mCCR8 |
|---|---|---|
| 11F5 | 0.207 | 0.168 |
| 11F5-N3S-11F5 | 0.168 | 0.119 |
| 11F5-Y5V-11F5 | 0.714 | 0.762 |
| 11F5-Y6N-11F5 | 0.254 | 0.198 |
| 11F5-N3S-Y5V-11F5 | 0.228 | 0.129 |
| 11F5-N3S-Y6N-11F5 | 0.174 | 0.105 |
| 11F5-Y5V-Y6N-11F5 | 0.592 | 0.546 |
| 11F5-N3S-Y5V-Y6N-11F5 | 0.210 | 0.141 |

The results showed that anti-CCR8 antibodies 1 1F5 and its CDR3-modified antibodies can bind to human CCR8 and mouse CCR8 with a high affinity.

### Example 13. Antibody-Dependent Cellular Cytotoxicity (ADCC) Determination of 11F5 and its CDR3-Modified Antibodies

The ADCC activity of 11F5 and its CDR3-modified antibodies were determined. The results are shown in the table below.

Specifically, engineered Jurkat cells were used as effector cells (Jurkat-Lue-hCD16A-158V), and H293T cells with hCCR8 surface antigen were used as target cells. The cells were incubated with each antibody at a ratio of 4:1 for 6 hours to determine ADCC activity. The results are shown in the table below.

TABLE 20
ADCC activity

| Antibody | EC50 (μg/mL) |
|---|---|
| 11F5 | 0.0108 |
| 11F5-N3S-11F5 | 0.0057 |
| 11F5-Y5V-11F5 | 0.0296 |
| 11F5-Y6N-11F5 | 0.0099 |
| 11F5-N3S-Y5V-11F5 | 0.0073 |
| 11F5-N3S-Y6N-11F5 | 0.0064 |
| 11F5-Y5V-Y6N-11F5 | 0.0362 |
| 11F5-N3S-Y5V-Y6N-11F5 | 0.0080 |

The results showed that 1 1F5 and all CDR3-modified antibodies exhibited a strong ADCC activity.

In another similar experiment, 11F5 and its CDR3-modified antibodies with SI mutations in the Fc region were also determined. The results are shown in the table below which indicates that 11F5 and its CDR3-modified antibodies exhibited a stronger ADCC activity when introduced SI mutations in the Fc region.

TABLE 21
ADCC activity

| Antibody | EC50 (μg/mL) |
|---|---|
| 11F5-SI | 0.0022 |
| 11F5-N3S-11F5-SI | 0.0007 |
| 11F5-N3S-Y5V-11F5-SI | 0.0014 |
| 11F5-N3S-Y6N-11F5-SI | 0.0017 |
| 11F5-N3S-Y5V-Y6N-11F5-SI | 0.0011 |
| 11F5 | 0.0337 |
| 11F5-N3S-11F5 | 0.0173 |
| 11F5-N3S-Y5V-11F5 | 0.0124 |
| 11F5-N3S-Y6N-11F5 | 0.0107 |
| 11F5-N3S-Y5V-Y6N-11F5 | 0.0102 |

### Example 14. Biophysical Analysis and Stability of 11F5 and/or its CDR3-Modified Antibodies

The biophysical properties of 11F5 and its CDR3-modified antibodies were evaluated. Similar experiments described in Example 6 were carried out. In addition, the colloidal stability detection using Standup Monolayer Chromatography (SMAC) method (indicated as the retention time of the main peak (SMAC, min) and cross-interaction detection with human serum polyclonal antibodies using Cross-Interaction Chromatography(CIC) method (indicated as the retention time of the main peak (CIC, min) were performed.

In the SAMC experiments, an Agilent 1260 chromatograph system (connected with Zenix™ column (4.6 mm×30 cm, Sepax, Cat #: 213300-4630)) was used, and samples were diluted to 1 mg/mL using PBS. The following parameters were used: mobile phase: 150 mM sodium phosphate (pH 7.0); flow rate: 0.35 mL/min; column temperature: 30 °C; detection wavelength: 280 nm, 220 nm; running time: 25 minutes; and injection mass: 2 μg.

In the CIC experiments, an Agilent 1260 chromatograph system (connected with 1 mL HiTrap® (GE Healthcare, Cat #: 17-0716-01) coupled with human serum polyclonal antibodies (Sigma, Cat #: 14506) was used, and samples were diluted to 1 mg/mL using PBS. The following parameters were used: mobile phase: PBS (pH 7.2-7.4); flow rate: 0.1 mL/min; detection wavelength: 280 nm, 220 nm; running time: 50 minutes; and injection mass: 10 μg.

TABLE 22
Biophysical properties test results

| | SEC | | | | | | | | |
|---|---|---|---|---|---|---|---|---|---|
| | Purity | | | CIC | SMAC | Tm (°C) | | | Tagg |
| Antibody | (%) | pI | HIC (min) | (min) | (min) | Tm1 | Tm2 | Tm3 | (°C) |
| 11F5 | 97.45 | 8.894 | 8.449/10.442/12.650 | 9.510 | 9.749 | 69.60 | 79.50 | — | 70.70 |
| 11F5-N3S-11F5 | 100.00 | 8.902 | 13.723 | 9.911 | 10.097 | 70.50 | 80.00 | — | 71.66 |
| 11F5-Y5V-11F5 | 99.23 | 8.902 | 3.168/4.590/6.600 | 8.662 | 8.217 | 69.80 | 78.10 | — | 70.90 |
| 11F5-Y6N-11F5 | 97.85 | 8.919 | 6.291 | 8.997 | 8.383 | 70.10 | 78.40 | — | 70.95 |
| 11F5-N3S-Y5V- | 99.44 | 8.910 | 7.262 | 8.718 | 8.232 | 70.40 | 79.49 | — | 71.36 |
| 11F5 | | | | | | | | | |
| 11F5-N3S-Y6N- | 98.03 | 8.930 | 6.545 | 9.160 | 8.503 | 70.60 | 79.90 | — | 71.95 |
| 11F5 | | | | | | | | | |
| 11F5-Y5V-Y6N- | 98.81 | 8.938 | 3.108 | 8.497 | 7.877 | 69.70 | 78.30 | — | 70.43 |
| 11F5 | | | | | | | | | |
| 11F5-N3S-Y5V- | 98.87 | 8.949 | 3.275 | 8.573 | 7.911 | 70.50 | 80.00 | — | 71.93 |
| Y6N-11F5 | | | | | | | | | |

The results showed that these 11F5's CDR3-modified antibodies exhibited good biophysical properties.

In another experiment, the stability of 11F5-N3S-Y5V-11F5-SI and 11F5-N3S-Y5V-Y6N-11F5-SI were evaluated. The results are shown in the table below which showed that 11F5-N3S-Y5V-11F5-SI and 11F5-N3S-Y5V-Y6N-11F5-SI exhibited good stability.

TABLE 23
Stability of 11F5's CDR3-modified antibodies

| | | | | CE-SDS (NR) | | | | | | | | | | |
|---|---|---|---|---|---|---|---|---|---|---|---|---|---|---|
| | | SEC | | | NG- | | | | | | cIEF | | | |
| | | Purity | HIC | Purity | IgG | HHL | HH | HL | HC | LC | Acidic | Main | Alkali | |
| Antibody | Treatment | (%) | (min) | (%) | (%) | (%) | (%) | (%) | (%) | (%) | (%) | (%) | (%) | pI |
| 11F5- | 0 d | 97.57 | 8.211 | 98.58 | — | 0.81 | — | 0.81 | — | 0.22 | 0 | 85.17 | 14.82 | 8.771 |
| N3S- | 4 °C 14 d | 98.88 | 8.211 | 98.52 | — | 0.90 | — | 0.37 | — | 0.21 | 0 | 84.81 | 15.19 | 8.770 |
| Y5V- | 40 °C 14 d | 98.25 | 8.228 | 91.05 | 6.40 | 0.97 | 0.40 | 0.40 | 0.15 | 0.29 | 29.82 | 59.70 | 10.49 | 8.775 |
| 11F5-SI | NH₄HCO₃ 6 h | 98.53 | 8.218 | 95.29 | 2.48 | 0.76 | 0.69 | 0.44 | — | 0.35 | 18.77 | 68.34 | 12.90 | 8.902 |
| | NH₄HCO₃ 24 h | 98.54 | 8.212 | 98.42 | — | 1.16 | — | 0.23 | — | 0.18 | 29.21 | 58.99 | 11.8 | 8.899 |
| | pH 3.5 0 h | 94.98 | 8.235 | 99.32 | — | 0.46 | — | 0.22 | — | — | 0 | 84.96 | 15.04 | 8.871 |
| | pH 3.5 6 h | 95.33 | 8.236 | 97.44 | — | 2.04 | — | 0.36 | — | 0.16 | 0 | 84.57 | 15.43 | 8.882 |
| 11F5- | 0 d | 96.87 | 3.703 | 97.76 | — | 1.53 | — | 0.51 | — | 0.20 | 0 | 83.63 | 16.37 | 8.794 |
| N3S- | 4 °C 14 d | 97.46 | 3.700 | 98.67 | — | 0.77 | — | 0.36 | — | 0.20 | 0 | 83.76 | 16.24 | 8.798 |
| Y5V- | 40 °C 14 d | 97.20 | 3.094 | 93.24 | — | 4.80 | 0.56 | 0.37 | 0.19 | 0.31 | 27.50 | 61.43 | 10.98 | 8.795 |
| Y6N- | NH₄HCO₃ 6 h | 97.71 | 3.700 | 98.36 | — | 0.84 | 0.24 | 0.36 | — | 0.21 | 18.14 | 67.93 | 13.93 | 8.919 |
| 11F5-SI | NH₄HCO₃ 24 h | 97.36 | 3.690 | 97.33 | — | 1.28 | 0.67 | 0.33 | — | 0.40 | 27.52 | 29.06 | 13.42 | 8.921 |
| | pH 3.5 0 h | 94.82 | 3.723 | 99.12 | — | 0.66 | — | 0.22 | — | — | 0 | 84.36 | 15.64 | 8.915 |
| | pH 3.5 6 h | 95.42 | 3.726 | 99.49 | — | 0.15 | — | — | — | — | 0 | 83.86 | 16.13 | 8.912 |

### Example 15. Blockade of Calcium Flux on hCCR8-Expressing Cells by 11F5 and its CDR3-Modified Antibodies

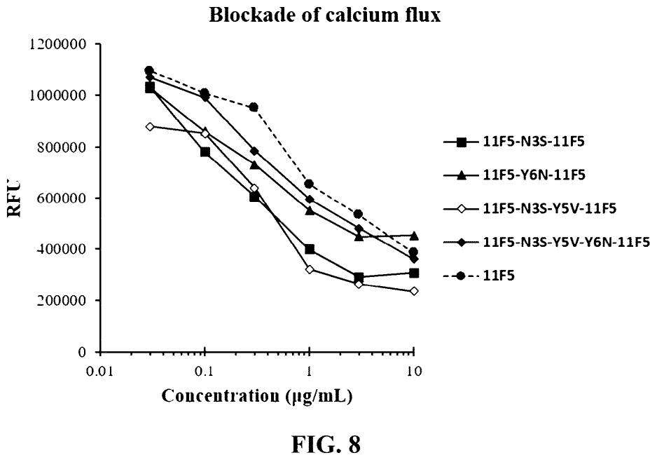

Blockade of hCCL1 binding to hCCR8 by 1 1F5 and its CDR3-modified antibodies were tested by conducting calcium (Ca) flux assays using human CCR8-expressing 293T cells. Fluorescent Imaging Plate Reader (FLIPR) Calcium 6 Assay Kit (Molecular Devices, Cat #: R8190) was used. Cells were seeded the day prior to the experiment and incubated overnight at 37 °C, 5% CO₂. The cells were then loaded with FLIPR Calcium 6 dye (Molecular Devices) including probenecid (Invitrogen) and PowerLoad (Invitrogen), and incubated for 30 minutes at 37 °C. Anti-CCR8 antibodies were added to the cells and further incubated at 37 °C for 30 minutes. After incubation, the Ca flux fluorescent signal induced by 1 μg/mL of recombinant human CCL1 (BioLegend, Cat #: 582706) was measured using SpectraMax® iD3 (Molecular Devices) for 1 minute. The results are showed in FIG. 8 and the table below.

TABLE 24
Blockade of calcium flux

| Antibody | IC50 (nM) | Max inhibition (%) |
|---|---|---|
| 11F5 | 5.573 | 82 |
| 11F5-N3S-11F5 | 0.787 | 93 |
| 11F5-Y6N-11F5 | 1.314 | 74 |
| 11F5-N3S-Y5V-11F5 | 2.533 | 96 |
| 11F5-N3S-Y5V-Y6N-11F5 | 2.898 | 85 |

The results showed that in the calcium influx blocking experiment, 11F5's CDR3-modified antibodies 11F5-N3S-11F5, 11F5-Y6N-11F5, 11F5-N3S-Y5V-11F5, and 11F5-N3S-Y5V-Y6N-11F5 exhibited superior blocking activity compared to 11F5. Particularly, 11F5-N3S-Y5V-11F5 demonstrated almost complete inhibition.

### Example 16. Anti-Tumor Activity of 11F5 and its CDR3-Modified Antibodies

11F5 and its CDR3-modified antibodies were tested for their effects on tumor growth in vivo in a colon cancer model. About 5×10⁵ MC38 cells were injected subcutaneously in each B-hCCR8 mouse. When the tumors in the mice reached a volume of about 150 mm³, the mice were randomly placed into different groups based on tumor volumes. The mice were then injected with phosphate buffer saline (PBS), 11F5 or its CDR3-modified antibodies by intraperitoneal (i.p.) administration.

The injected volume was calculated based on the weight of the mouse and desired dosage of 3 mg/kg (Details are shown in the table below).

TABLE 25
Group assignment

| | No. of | | | | | Total No. of |
|---|---|---|---|---|---|---|
| Group | mice | Antibodies | Dosage | Route | Frequency | administration |
| G1 | 6 | PBS | — | i.p. | BIW | 6 |
| G2 | 6 | 11F5-N3S-Y5V-11F5-SI | 3 mg/kg | i.p. | BIW | 6 |
| G3 | 6 | 11F5-SI | 3 mg/kg | i.p. | BIW | 6 |

The table below summarizes the results for this experiment, including the tumor volumes on the day of grouping (Day 0), 14 days after the grouping (Day 14), and on the 21 days of the experiment (Day 21); the survival rate of the mice; Tumor Growth Inhibition value (TGI); and the statistical differences (P value) of tumor volume and body weight between the treatment and control groups.

TABLE 26
Tumor size changes

| | | | | | | P value | |
|---|---|---|---|---|---|---|---|
| | | Tumor volume (mm³) | | | TGI | Body | Tumor |
| Group | Antibodies | Day 0 | Day 14 | Day 21 | % | weight | Volume |
| G1 | PBS | 152 ± 7 | 1304 ± 177 | 2078 ± 280 | N/A | N/A | N/A |
| G2 | 11F5-N3S-Y5V-11F5-SI | 151 ± 9 | 527 ± 99 | 619 ± 130 | 75.7 | 0.076 | 0.001 |
| G3 | 11F5-SI | 152 ± 9 | 7381 ± 63 | 1281 ± 450 | 41.3 | 0.201 | 0.164 |

The results showed that 11F5-N3S-Y5V-11F5-SI exhibited a better tumor growth inhibition effect than 11F5-SI.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDRs 1, 2, 3 amino acid sequences are set forth in SEQ ID NOs: 1, 2, 3, respectively,
(2) the selected VH CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively;
(4) the selected VH CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively;
(6) the selected VH CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 72, 73, 74, respectively;
(8) the selected VH CDRs 1, 2, 3 amino acid sequences are set forth in SEQ ID NOs: 75, 76, 77, respectively;
(9) the selected VH CDRs 1, 2, 3 amino acid sequences are set forth in SEQ ID NOs: 78, 79, 80, respectively;
(10) the selected VH CDRs 1, 2, 3 amino acid sequences are set forth in SEQ ID NOs: 81, 82, 83, respectively;
(11) the selected VH CDRs 1, 2, 3 amino acid sequences are set forth in SEQ ID NOs: 84, 85, 86, respectively;
(12) the selected VH CDRs 1, 2, 3 amino acid sequences are set forth in SEQ ID NOs: 87, 88, 89, respectively;
(13) the selected VH CDRs 1, 2, 3 amino acid sequences are set forth in SEQ ID NOs: 90, 91, 92, respectively;
(14) the selected VH CDRs 1, 2, 3 amino acid sequences are set forth in SEQ ID NOs: 93, 94, 95, respectively;
(15) the selected VH CDRs 1, 2, 3 amino acid sequences are set forth in SEQ ID NOs: 96, 97, 98, respectively;
(16) the selected VH CDRs 1, 2, 3 amino acid sequences are set forth in SEQ ID NOs: 144, 145, 146, respectively;
(17) the selected VH CDRs 1, 2, 3 amino acid sequences are set forth in SEQ ID NOs: 147, 148, 149, respectively;
(18) the selected VH CDRs 1, 2, 3 amino acid sequences are set forth in SEQ ID NOs: 150, 151, 152, respectively;
(19) the selected VH CDRs 1, 2, 3 amino acid sequences are set forth in SEQ ID NOs: 153, 154, 155, respectively;
(20) the selected VH CDRs 1, 2, 3 amino acid sequences are set forth in SEQ ID NOs: 156, 157, 158, respectively;
(21) the selected VH CDRs 1, 2, 3 amino acid sequences are set forth in SEQ ID NOs: 159, 160, 161, respectively;
(22) the selected VH CDRs 1, 2, 3 amino acid sequences are set forth in SEQ ID NOs: 162, 163, 164, respectively;
(23) the selected VH CDRs 1, 2, 3 amino acid sequences are set forth in SEQ ID NOs: 165, 166, 167, respectively;
(24) the selected VH CDRs 1, 2, 3 amino acid sequences are set forth in SEQ ID NOs: 168, 169, 170, respectively;
(25) the selected VH CDRs 1, 2, 3 amino acid sequences are set forth in SEQ ID NOs: 219, 220, 221, respectively;
(26) the selected VH CDRs 1, 2, 3 amino acid sequences are set forth in SEQ ID NOs: 222, 223, 224, respectively;
(27) the selected VH CDRs 1, 2, 3 amino acid sequences are set forth in SEQ ID NOs: 225, 226, 227, respectively;
(28) the selected VH CDRs 1, 2, 3 amino acid sequences are set forth in SEQ ID NOs: 228, 229, 230, respectively;
(29) the selected VH CDRs 1, 2, 3 amino acid sequences are set forth in SEQ ID NOs: 231, 232, 233, respectively;
(30) the selected VH CDRs 1, 2, 3 amino acid sequences are set forth in SEQ ID NOs: 234, 235, 236, respectively;
(31) the selected VH CDRs 1, 2, 3 amino acid sequences are set forth in SEQ ID NOs: 237, 238, 239, respectively;
(32) the selected VH CDRs 1, 2, 3 amino acid sequences are set forth in SEQ ID NOs: 240, 241, 242, respectively;
(33) the selected VH CDRs 1, 2, 3 amino acid sequences are set forth in SEQ ID NOs: 243, 244, 245, respectively;
(34) the selected VH CDRs 1, 2, 3 amino acid sequences are set forth in SEQ ID NOs: 246, 247, 248, respectively;
(35) the selected VH CDRs 1, 2, 3 amino acid sequences are set forth in SEQ ID NOs: 249, 250, 251, respectively;
(36) the selected VH CDRs 1, 2, 3 amino acid sequences are set forth in SEQ ID NOs: 252, 253, 254, respectively;
(37) the selected VH CDRs 1, 2, 3 amino acid sequences are set forth in SEQ ID NOs: 255, 256, 257, respectively; and
(38) the selected VH CDRs 1, 2, 3 amino acid sequences are set forth in SEQ ID NOs: 258, 259, 260, respectively;
wherein the selected VL CDRs, 1, 2, and 3 amino acid sequences are one of the following:
(1) the selected VL CDRs 1, 2, 3 amino acid sequences are set forth in SEQ ID NOs: 19, 20, 21, respectively;
(2) the selected VL CDRs 1, 2, 3 amino acid sequences are set forth in SEQ ID NOs: 22, 23, 24, respectively;
(3) the selected VL CDRs 1, 2, 3 amino acid sequences are set forth in SEQ ID NOs: 25, 26, 27, respectively;
(4) the selected VL CDRs 1, 2, 3 amino acid sequences are set forth in SEQ ID NOs: 28, 29, 30, respectively;
(5) the selected VL CDRs 1, 2, 3 amino acid sequences are set forth in SEQ ID NOs: 31, 32, 33, respectively;
(6) the selected VL CDRs 1, 2, 3 amino acid sequences are set forth in SEQ ID NOs: 34, 35, 36, respectively;
(7) the selected VL CDRs 1, 2, 3 amino acid sequences are set forth in SEQ ID NOs: 108, 109, 110, respectively;
(8) the selected VL CDRs 1, 2, 3 amino acid sequences are set forth in SEQ ID NOs: 111, 112, 113, respectively;
(9) the selected VL CDRs 1, 2, 3 amino acid sequences are set forth in SEQ ID NOs: 114, 115, 116, respectively;
(10) the selected VL CDRs 1, 2, 3 amino acid sequences are set forth in SEQ ID NOs: 117, 118, 119, respectively;
(11) the selected VL CDRs 1, 2, 3 amino acid sequences are set forth in SEQ ID NOs: 120, 121, 122, respectively;
(12) the selected VL CDRs 1, 2, 3 amino acid sequences are set forth in SEQ ID NOs: 123, 124, 125, respectively;
(13) the selected VL CDRs 1, 2, 3 amino acid sequences are set forth in SEQ ID NOs: 126, 127, 128, respectively;
(14) the selected VL CDRs 1, 2, 3 amino acid sequences are set forth in SEQ ID NOs: 129, 130, 131, respectively;
(15) the selected VL CDRs 1, 2, 3 amino acid sequences are set forth in SEQ ID NOs: 132, 133, 134, respectively;
(16) the selected VL CDRs 1, 2, 3 amino acid sequences are set forth in SEQ ID NOs: 180, 181, 182, respectively;
(17) the selected VL CDRs 1, 2, 3 amino acid sequences are set forth in SEQ ID NOs: 183, 184, 185, respectively;
(18) the selected VL CDRs 1, 2, 3 amino acid sequences are set forth in SEQ ID NOs: 186, 187, 188, respectively;
(19) the selected VL CDRs 1, 2, 3 amino acid sequences are set forth in SEQ ID NOs: 189, 190, 191, respectively;
(20) the selected VL CDRs 1, 2, 3 amino acid sequences are set forth in SEQ ID NOs: 192, 193, 194, respectively;
(21) the selected VL CDRs 1, 2, 3 amino acid sequences are set forth in SEQ ID NOs: 195, 196, 197, respectively;
(22) the selected VL CDRs 1, 2, 3 amino acid sequences are set forth in SEQ ID NOs: 198, 199, 200, respectively;
(23) the selected VL CDRs 1, 2, 3 amino acid sequences are set forth in SEQ ID NOs: 201, 202, 203, respectively; and
(24) the selected VL CDRs 1, 2, 3 amino acid sequences are set forth in SEQ ID NOs: 204, 205, 206, respectively.

2. An antibody or antigen-binding fragment thereof that binds to CCR8 (chemokine (C-C motif) receptor 8), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences are one of the following:
(1) the selected VH CDR 1 is SYVMH (SEQ ID NO: 99); the selected VH CDR2 is VISYX₁X₂SDKYYADSVKG (SEQ ID NO: 100); the selected VH CDR3 is GRGYRYGQSYYYGMDV (SEQ ID NO: 101);
(2) the selected VH CDR 1 is SYVVH (SEQ ID NO: 102); the selected VH CDR2 is VISYX₁X₂DNKFYADSVKG (SEQ ID NO: 103); the selected VH CDR3 is GRX₅YX₆X₇YYGLDV (SEQ ID NO: 104);
(3) the selected VH CDR 1 is TYVMH (SEQ ID NO: 105); the selected VH CDR2 is VISYX₁X₂NNKYYADSVKG (SEQ ID NO: 106); the selected VH CDR3 is GRSYVNYYGLDV (SEQ ID NO: 107);
(4) the selected VH CDR 1 is GFSFSSY (SEQ ID NO: 171); the selected VH CDR2 is SYX₁X₂SD (SEQ ID NO: 172); the selected VH CDR3 is GRGYRYGQSYYYGMDV (SEQ ID NO: 173);
(5) the selected VH CDR 1 is GFTFSSY (SEQ ID NO: 174); the selected VH CDR2 is SYX₁X₂DN (SEQ ID NO: 175); the selected VH CDR3 is GRX₅YX₆X₇YYGLDV (SEQ ID NO: 176); and
(6) the selected VH CDR 1 is GFTFSTY (SEQ ID NO: 177); the selected VH CDR2 is SYX₁X₂NN (SEQ ID NO: 178); the selected VH CDR3 is GRSYVNYYGLDV (SEQ ID NO: 179);
wherein the selected VL CDRs 1, 2, and 3 amino acid sequences are one of the following:
(1) the selected VL CDR 1 is KSSQSLLYSX₃X₄KTYLY (SEQ ID NO: 135); the selected VL CDR2 is EVSNRFS (SEQ ID NO: 136); the selected VL CDR3 is MQSIKLPLT (SEQ ID NO: 137);
(2) the selected VL CDR 1 is KSSQSLLYSX₃X₄KTYLY (SEQ ID NO: 138); the selected VL CDR2 is EVSNRFS (SEQ ID NO: 139); the selected VL CDR3 is MQSLKVPPT (SEQ ID NO: 140);
(3) the selected VL CDR 1 is KSSQSLLYSX₃X₄KTYLY (SEQ ID NO: 141); the selected VL CDR2 is EVSNRFS (SEQ ID NO: 142); the selected VL CDR3 is MQSVKIPLT (SEQ ID NO: 143);
(4) the selected VL CDR 1 is KSSQSLLYSX₃X₄KTYLY (SEQ ID NO: 207); the selected VL CDR2 is EVSNRFS (SEQ ID NO: 208); the selected VL CDR3 is MQSIKLPLT (SEQ ID NO: 209);
(5) the selected VL CDR 1 is KSSQSLLYSX₃X₄KTYLY (SEQ ID NO: 210); the selected VL CDR2 is EVSNRFS (SEQ ID NO: 211); the selected VL CDR3 is MQSLKVPPT (SEQ ID NO: 212); and
(6) the selected VL CDR 1 is KSSQSLLYSX₃X₄KTYLY (SEQ ID NO: 213); the selected VL CDR2 is EVSNRFS (SEQ ID NO: 214); the selected VL CDR3 is MQSVKIPLT (SEQ ID NO: 215);
wherein X₁and X₃ in each sequence are independently selected from the group consisting of D, S, and A,
wherein X₂ and X₄ in each sequence are independently selected from the group consisting of G and A,
wherein X₅ in SEQ ID NO: 104 or 176 is independently selected from the group consisting of N and S,
wherein X₆ in SEQ ID NO: 104 or 176 is independently selected from the group consisting of Y and V,
wherein X₇ in SEQ ID NO: 104 or 176 is independently selected from the group consisting of Y and N.

3. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 19, 20 and 21, respectively according to Kabat definition;
(2) the VH comprises CDRs 1, 2, 3 with the amino acid sequences set forth in SEQ ID NOs: 4, 5 and 6, respectively, and the VL comprises CDRs 1, 2, 3 with the amino acid sequences set forth in SEQ ID NOs: 22, 23 and 24, respectively according to Kabat definition;
(3) the VH comprises CDRs 1, 2, 3 with the amino acid sequences set forth in SEQ ID NOs: 7, 8 and 9, respectively, and the VL comprises CDRs 1, 2, 3 with the amino acid sequences set forth in SEQ ID NOs: 25, 26 and 27, respectively according to Kabat definition;
(4) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 28, 29, and 30, respectively according to Chothia definition;
(5) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 31, 32, and 33, respectively according to Chothia definition;
(6) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 34, 35, and 36, respectively according to Chothia definition;
(7) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 22, 23 and 24, respectively according to Kabat definition;
(8) the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 25, 26 and 27, respectively according to Kabat definition;
(9) the VH comprises CDRs 1, 2, 3 with the amino acid sequences set forth in SEQ ID NOs: 4, 5, and 6 respectively, and the VL comprises CDRs 1, 2, 3 with the amino acid sequences set forth in SEQ ID NOs: 19, 20 and 21, respectively according to Kabat definition;
(10) the VH comprises CDRs 1, 2, 3 with the amino acid sequences set forth in SEQ ID NOs: 4, 5, and 6 respectively, and the VL comprises CDRs 1, 2, 3 with the amino acid sequences set forth in SEQ ID NOs: 25, 26, and 27, respectively according to Kabat definition;
(11) the VH comprises CDRs 1, 2, 3 with the amino acid sequences set forth in SEQ ID NOs: 7, 8 and 9 respectively, and the VL comprises CDRs 1, 2, 3 with the amino acid sequences set forth in SEQ ID NOs: 19, 20 and 21, respectively according to Kabat definition;
(12) the VH comprises CDRs 1, 2, 3 with the amino acid sequences set forth in SEQ ID NOs: 7, 8, and 9 respectively, and the VL comprises CDRs 1, 2, 3 with the amino acid sequences set forth in SEQ ID NOs: 22, 23, and 24, respectively according to Kabat definition;
(13) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 31, 32, and 33, respectively according to Chothia definition;
(14) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 34, 35, and 36, respectively according to Chothia definition;
(15) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 28, 29, and 30, respectively according to Chothia definition;
(16) the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 34, 35, and 36, respectively according to Chothia definition;
(17) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 28, 29, and 30, respectively according to Chothia definition; and
(18) the VH comprises CDRs 1, 2, 3 with the amino acid sequences set forth in SEQ ID NOs: 16, 17, and 18, respectively, and the VL comprises CDRs 1, 2, 3 with the amino acid sequences set forth in SEQ ID NOs: 31, 32, and 33, respectively according to Chothia definition.

4. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the VH comprises CDRs 1, 2, 3 with the amino acid sequences set forth in SEQ ID NOs: 1, 2, and 3, respectively, and the VL comprises CDRs 1, 2, 3 with the amino acid sequences set forth in SEQ ID NOs: 114, 115, and 116, respectively;
(2) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 114, 115, and 116, respectively;
(3) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 108, 109, and 110, respectively;
(4) the VH comprises CDRs 1, 2, 3 with the amino acid sequences set forth in SEQ ID NOs: 78, 79, and 80, respectively, and the VL comprises CDRs 1, 2, 3 with the amino acid sequences set forth in SEQ ID NOs: 114, 115, and 116, respectively;
(5) the VH comprises CDRs 1, 2, 3 with the amino acid sequences set forth in SEQ ID NOs: 75, 76, and 77, respectively, and the VL comprises CDRs 1, 2, 3 with the amino acid sequences set forth in SEQ ID NOs: 111, 112, and 113, respectively;
(6) the VH comprises CDRs 1, 2, 3 with the amino acid sequences set forth in SEQ ID NOs: 10, 11, and 12, respectively, and the VL comprises CDRs 1, 2, 3 with the amino acid sequences set forth in SEQ ID NOs: 186, 187, and 188, respectively;
(7) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 186, 187, and 188, respectively;
(8) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 180, 181, and 182, respectively;
(9) the VH comprises CDRs 1, 2, 3 with the amino acid sequences set forth in SEQ ID NOs: 150, 151, and 152, respectively, and the VL comprises CDRs 1, 2, 3 with the amino acid sequences set forth in SEQ ID NOs: 186, 187, and 188, respectively;
(10) the VH comprises CDRs 1, 2, 3 with the amino acid sequences set forth in SEQ ID NOs: 147, 148, and 149, respectively, and the VL comprises CDRs 1, 2, 3 with the amino acid sequences set forth in SEQ ID NOs: 183, 184, and 185, respectively;
(11) the VH comprises CDRs 1, 2, 3 with the amino acid sequences set forth in SEQ ID NOs: 72, 73, and 74, respectively, and the VL comprises CDRs 1, 2, 3 with the amino acid sequences set forth in SEQ ID NOs: 25, 26, and 27, respectively;
(12) the VH comprises CDRs 1, 2, 3 with the amino acid sequences set forth in SEQ ID NOs: 144, 145, and 146, respectively, and the VL comprises CDRs 1, 2, 3 with the amino acid sequences set forth in SEQ ID NOs: 34, 35, and 36, respectively;
(13) the VH comprises CDRs 1, 2, 3 with the amino acid sequences set forth in SEQ ID NOs: 219, 220, and 221, respectively, and the VL comprises CDRs 1, 2, 3 with the amino acid sequences set forth in SEQ ID NOs: 22, 23, and 24, respectively;
(14) the VH comprises CDRs 1, 2, 3 with the amino acid sequences set forth in SEQ ID NOs: 222, 223, and 224, respectively, and the VL comprises CDRs 1, 2, 3 with the amino acid sequences set forth in SEQ ID NOs: 22, 23, and 24, respectively;
(15) the VH comprises CDRs 1, 2, 3 with the amino acid sequences set forth in SEQ ID NOs: 225, 226, and 227, respectively, and the VL comprises CDRs 1, 2, 3 with the amino acid sequences set forth in SEQ ID NOs: 22, 23, and 24, respectively;
(16) the VH comprises CDRs 1, 2, 3 with the amino acid sequences set forth in SEQ ID NOs: 228, 229, and 230, respectively, and the VL comprises CDRs 1, 2, 3 with the amino acid sequences set forth in SEQ ID NOs: 22, 23, and 24, respectively;
(17) the VH comprises CDRs 1, 2, 3 with the amino acid sequences set forth in SEQ ID NOs: 231, 232, and 233, respectively, and the VL comprises CDRs 1, 2, 3 with the amino acid sequences set forth in SEQ ID NOs: 22, 23, and 24, respectively;
(18) the VH comprises CDRs 1, 2, 3 with the amino acid sequences set forth in SEQ ID NOs: 234, 235, and 236, respectively, and the VL comprises CDRs 1, 2, 3 with the amino acid sequences set forth in SEQ ID NOs: 22, 23, and 24, respectively;
(19) the VH comprises CDRs 1, 2, 3 with the amino acid sequences set forth in SEQ ID NOs: 237, 238, and 239, respectively, and the VL comprises CDRs 1, 2, 3 with the amino acid sequences set forth in SEQ ID NOs: 22, 23, and 24, respectively;
(20) the VH comprises CDRs 1, 2, 3 with the amino acid sequences set forth in SEQ ID NOs: 240, 241, and 242, respectively, and the VL comprises CDRs 1, 2, 3 with the amino acid sequences set forth in SEQ ID NOs: 31, 32, and 33, respectively;
(21) the VH comprises CDRs 1, 2, 3 with the amino acid sequences set forth in SEQ ID NOs: 243, 244, and 245, respectively, and the VL comprises CDRs 1, 2, 3 with the amino acid sequences set forth in SEQ ID NOs: 31, 32, and 33, respectively;
(22) the VH comprises CDRs 1, 2, 3 with the amino acid sequences set forth in SEQ ID NOs: 246, 247, and 248, respectively, and the VL comprises CDRs 1, 2, 3 with the amino acid sequences set forth in SEQ ID NOs: 31, 32, and 33, respectively;
(23) the VH comprises CDRs 1, 2, 3 with the amino acid sequences set forth in SEQ ID NOs: 249, 250, and 251, respectively, and the VL comprises CDRs 1, 2, 3 with the amino acid sequences set forth in SEQ ID NOs: 31, 32, and 33, respectively;
(24) the VH comprises CDRs 1, 2, 3 with the amino acid sequences set forth in SEQ ID NOs: 252, 253, and 254, respectively, and the VL comprises CDRs 1, 2, 3 with the amino acid sequences set forth in SEQ ID NOs: 31, 32, and 33, respectively;
(25) the VH comprises CDRs 1, 2, 3 with the amino acid sequences set forth in SEQ ID NOs: 255, 256, and 257, respectively, and the VL comprises CDRs 1, 2, 3 with the amino acid sequences set forth in SEQ ID NOs: 31, 32, and 33, respectively; and
(26) the VH comprises CDRs 1, 2, 3 with the amino acid sequences set forth in SEQ ID NOs: 258, 259, and 260, respectively, and the VL comprises CDRs 1, 2, 3 with the amino acid sequences set forth in SEQ ID NOs: 31, 32, and 33, respectively.

5. The antibody or antigen-binding fragment thereof of any one of claims 1-4, wherein the antibody or antigen-binding fragment specifically binds to human CCR8, mouse CCR8 or monkey CCR8.

6. The antibody or antigen-binding fragment thereof of any one of claims 1-5, wherein the antibody or antigen-binding fragment is a human or humanized antibody or antigen-binding fragment thereof (e.g., a human IgG1 antibody).

7. The antibody or antigen-binding fragment thereof of any one of claims 1-6, wherein the antibody or antigen-binding fragment is a single-chain variable fragment (scFv), a one-armed antibody, or a multi-specific antibody (e.g., a bispecific antibody).

8. A nucleic acid comprising a polynucleotide encoding a polypeptide comprising: an immunoglobulin heavy chain or a fragment thereof comprising a heavy chain variable region (VH) comprising complementarity determining regions (CDRs) 1, 2, and 3, and wherein the VH, when paired with a light chain variable region (VL) binds to CCR8,
wherein the VH CDRs 1, 2, and 3 amino acid sequences are one of the following:
(1) the selected VH CDRs 1, 2, 3 amino acid sequences are set forth in SEQ ID NOs: 1, 2, 3, respectively,
(2) the selected VH CDRs 1, 2, 3 amino acid sequences are set forth in SEQ ID NOs: 4, 5, 6, respectively;
(3) the selected VH CDRs 1, 2, 3 amino acid sequences are set forth in SEQ ID NOs: 7, 8, 9, respectively;
(4) the selected VH CDRs 1, 2, 3 amino acid sequences are set forth in SEQ ID NOs: 10, 11, 12, respectively;
(5) the selected VH CDRs 1, 2, 3 amino acid sequences are set forth in SEQ ID NOs: 13, 14, 15, respectively;
(6) the selected VH CDRs 1, 2, 3 amino acid sequences are set forth in SEQ ID NOs: 16, 17, 18, respectively;
(7) the selected VH CDRs 1, 2, 3 amino acid sequences are set forth in SEQ ID NOs: 72, 73, 74, respectively;
(8) the selected VH CDRs 1, 2, 3 amino acid sequences are set forth in SEQ ID NOs: 75, 76, 77, respectively;
(9) the selected VH CDRs 1, 2, 3 amino acid sequences are set forth in SEQ ID NOs: 78, 79, 80, respectively;
(10) the selected VH CDRs 1, 2, 3 amino acid sequences are set forth in SEQ ID NOs: 81, 82, 83, respectively;
(11) the selected VH CDRs 1, 2, 3 amino acid sequences are set forth in SEQ ID NOs: 84, 85, 86, respectively;
(12) the selected VH CDRs 1, 2, 3 amino acid sequences are set forth in SEQ ID NOs: 87, 88, 89, respectively;
(13) the selected VH CDRs 1, 2, 3 amino acid sequences are set forth in SEQ ID NOs: 90, 91, 92, respectively;
(14) the selected VH CDRs 1, 2, 3 amino acid sequences are set forth in SEQ ID NOs: 93, 94, 95, respectively;
(15) the selected VH CDRs 1, 2, 3 amino acid sequences are set forth in SEQ ID NOs: 96, 97, 98, respectively;
(16) the selected VH CDRs 1, 2, 3 amino acid sequences are set forth in SEQ ID NOs: 99, 100, 101, respectively;
(17) the selected VH CDRs 1, 2, 3 amino acid sequences are set forth in SEQ ID NOs: 102, 103, 104, respectively;
(18) the selected VH CDRs 1, 2, 3 amino acid sequences are set forth in SEQ ID NOs: 105, 106, 107, respectively;
(19) the selected VH CDRs 1, 2, 3 amino acid sequences are set forth in SEQ ID NOs: 144, 145, 146, respectively;
(20) the selected VH CDRs 1, 2, 3 amino acid sequences are set forth in SEQ ID NOs: 147, 148, 149, respectively;
(21) the selected VH CDRs 1, 2, 3 amino acid sequences are set forth in SEQ ID NOs: 150, 151, 152, respectively;
(22) the selected VH CDRs 1, 2, 3 amino acid sequences are set forth in SEQ ID NOs: 153, 154, 155, respectively;
(23) the selected VH CDRs 1, 2, 3 amino acid sequences are set forth in SEQ ID NOs: 156, 157, 158, respectively;
(24) the selected VH CDRs 1, 2, 3 amino acid sequences are set forth in SEQ ID NOs: 159, 160, 161, respectively;
(25) the selected VH CDRs 1, 2, 3 amino acid sequences are set forth in SEQ ID NOs: 162, 163, 164, respectively;
(26) the selected VH CDRs 1, 2, 3 amino acid sequences are set forth in SEQ ID NOs: 165, 166, 167, respectively;
(27) the selected VH CDRs 1, 2, 3 amino acid sequences are set forth in SEQ ID NOs: 168, 169, 170, respectively;
(28) the selected VH CDRs 1, 2, 3 amino acid sequences are set forth in SEQ ID NOs: 171, 172, 173, respectively;
(29) the selected VH CDRs 1, 2, 3 amino acid sequences are set forth in SEQ ID NOs: 174, 175, 176, respectively;
(30) the selected VH CDRs 1, 2, 3 amino acid sequences are set forth in SEQ ID NOs: 177, 178, 179, respectively;
(31) the selected VH CDRs 1, 2, 3 amino acid sequences are set forth in SEQ ID NOs: 219, 220, 221, respectively;
(32) the selected VH CDRs 1, 2, 3 amino acid sequences are set forth in SEQ ID NOs: 222, 223, 224, respectively;
(33) the selected VH CDRs 1, 2, 3 amino acid sequences are set forth in SEQ ID NOs: 225, 226, 227, respectively;
(34) the selected VH CDRs 1, 2, 3 amino acid sequences are set forth in SEQ ID NOs: 228, 229, 230, respectively;
(35) the selected VH CDRs 1, 2, 3 amino acid sequences are set forth in SEQ ID NOs: 231, 232, 233, respectively;
(36) the selected VH CDRs 1, 2, 3 amino acid sequences are set forth in SEQ ID NOs: 234, 235, 236, respectively;
(37) the selected VH CDRs 1, 2, 3 amino acid sequences are set forth in SEQ ID NOs: 237, 238, 239, respectively;
(38) the selected VH CDRs 1, 2, 3 amino acid sequences are set forth in SEQ ID NOs: 240, 241, 242, respectively;
(39) the selected VH CDRs 1, 2, 3 amino acid sequences are set forth in SEQ ID NOs: 243, 244, 245, respectively;
(40) the selected VH CDRs 1, 2, 3 amino acid sequences are set forth in SEQ ID NOs: 246, 247, 248, respectively;
(41) the selected VH CDRs 1, 2, 3 amino acid sequences are set forth in SEQ ID NOs: 249, 250, 251, respectively;
(42) the selected VH CDRs 1, 2, 3 amino acid sequences are set forth in SEQ ID NOs: 252, 253, 254, respectively;
(43) the selected VH CDRs 1, 2, 3 amino acid sequences are set forth in SEQ ID NOs: 255, 256, 257, respectively; and
(44) the selected VH CDRs 1, 2, 3 amino acid sequences are set forth in SEQ ID NOs: 258, 259, 260, respectively;
wherein the VL amino acid sequence is selected from the group consisting of: SEQ ID NOs: 40, 41, 42, 66, 67, and 68.

9. The nucleic acid of claim 8, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

10. The nucleic acid of claim 8, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4, 5 and 6, respectively.

11. The nucleic acid of claim 8, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8 and 9, respectively.

12. A nucleic acid comprising a polynucleotide encoding a polypeptide comprising: an immunoglobulin heavy chain or a fragment thereof comprising a light chain variable region (VL) comprising complementarity determining regions (CDRs) 1, 2, and 3, and wherein the VL, when paired with a heavy chain variable region (VH) binds to CCR8, wherein the selected VL CDRs, 1, 2, and 3 amino acid sequences are one of the following:
(1) the selected VL CDRs 1, 2, 3 amino acid sequences are set forth in SEQ ID NOs: 19, 20, 21, respectively;
(2) the selected VL CDRs 1, 2, 3 amino acid sequences are set forth in SEQ ID NOs: 22, 23, 24, respectively;
(3) the selected VL CDRs 1, 2, 3 amino acid sequences are set forth in SEQ ID NOs: 25, 26, 27, respectively;
(4) the selected VL CDRs 1, 2, 3 amino acid sequences are set forth in SEQ ID NOs: 28, 29, 30, respectively;
(5) the selected VL CDRs 1, 2, 3 amino acid sequences are set forth in SEQ ID NOs: 31, 32, 33, respectively;
(6) the selected VL CDRs 1, 2, 3 amino acid sequences are set forth in SEQ ID NOs: 34, 35, 36, respectively;
(7) the selected VL CDRs 1, 2, 3 amino acid sequences are set forth in SEQ ID NOs: 108, 109, 110, respectively;
(8) the selected VL CDRs 1, 2, 3 amino acid sequences are set forth in SEQ ID NOs: 111, 112, 113, respectively;
(9) the selected VL CDRs 1, 2, 3 amino acid sequences are set forth in SEQ ID NOs: 114, 115, 116, respectively;
(10) the selected VL CDRs 1, 2, 3 amino acid sequences are set forth in SEQ ID NOs: 117, 118, 119, respectively;
(11) the selected VL CDRs 1, 2, 3 amino acid sequences are set forth in SEQ ID NOs: 120, 121, 122, respectively;
(12) the selected VL CDRs 1, 2, 3 amino acid sequences are set forth in SEQ ID NOs: 123, 124, 125, respectively;
(13) the selected VL CDRs 1, 2, 3 amino acid sequences are set forth in SEQ ID NOs: 126, 127, 128, respectively;
(14) the selected VL CDRs 1, 2, 3 amino acid sequences are set forth in SEQ ID NOs: 129, 130, 131, respectively;
(15) the selected VL CDRs 1, 2, 3 amino acid sequences are set forth in SEQ ID NOs: 132, 133, 134, respectively;
(16) the selected VL CDRs 1, 2, 3 amino acid sequences are set forth in SEQ ID NOs: 135, 136, 137, respectively;
(17) the selected VL CDRs 1, 2, 3 amino acid sequences are set forth in SEQ ID NOs: 138, 139, 140, respectively;
(18) the selected VL CDRs 1, 2, 3 amino acid sequences are set forth in SEQ ID NOs: 141, 142, 143, respectively;
(19) the selected VL CDRs 1, 2, 3 amino acid sequences are set forth in SEQ ID NOs: 180, 181, 182, respectively;
(20) the selected VL CDRs 1, 2, 3 amino acid sequences are set forth in SEQ ID NOs: 183, 184, 185, respectively;
(21) the selected VL CDRs 1, 2, 3 amino acid sequences are set forth in SEQ ID NOs: 186, 187, 188, respectively;
(22) the selected VL CDRs 1, 2, 3 amino acid sequences are set forth in SEQ ID NOs: 189, 190, 191, respectively;
(23) the selected VL CDRs 1, 2, 3 amino acid sequences are set forth in SEQ ID NOs: 192, 193, 194, respectively;
(24) the selected VL CDRs 1, 2, 3 amino acid sequences are set forth in SEQ ID NOs: 195, 196, 197, respectively;
(25) the selected VL CDRs 1, 2, 3 amino acid sequences are set forth in SEQ ID NOs: 198, 199, 200, respectively;
(26) the selected VL CDRs 1, 2, 3 amino acid sequences are set forth in SEQ ID NOs: 201, 202, 203, respectively;
(27) the selected VL CDRs 1, 2, 3 amino acid sequences are set forth in SEQ ID NOs: 204, 205, 206, respectively;
(28) the selected VL CDRs 1, 2, 3 amino acid sequences are set forth in SEQ ID NOs: 207, 208, 209, respectively;
(29) the selected VL CDRs 1, 2, 3 amino acid sequences are set forth in SEQ ID NOs: 210, 211, 212, respectively; and
(30) the selected VL CDRs 1, 2, 3 amino acid sequences are set forth in SEQ ID NOs: 213, 214, 215, respectively,
wherein the VH amino acid sequence is selected from the group consisting of: SEQ ID NOs: 37, 38, 39, 63, 64, 65, 69, 70, 71, 216, 261, 262, 263, 264, 265, 266, and 267.

13. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 19, 20 and 21, respectively.

14. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 22, 23 and 24, respectively.

15. The nucleic acid of claim 12, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 25, 26 and 27, respectively.

16. The nucleic acid of any one of claims 8-15, wherein the VH when paired with a VL specifically binds to human CCR8, mouse CCR8 or monkey CCR8, or the VL when paired with a VH specifically binds to human CCR8, mouse CCR8 or monkey CCR8.

17. The nucleic acid of any one of claims 8-16, wherein the immunoglobulin heavy chain or the fragment thereof is a human or humanized immunoglobulin heavy chain or a fragment thereof, and the immunoglobulin light chain or the fragment thereof is a human or humanized immunoglobulin light chain or a fragment thereof.

18. The nucleic acid of any one of claims 8-17, wherein the nucleic acid encodes a single-chain variable fragment (scFv), a one-armed antibody, or a multi-specific antibody (e.g., a bispecific antibody).

19. The nucleic acid of any one of claims 8-18, wherein the nucleic acid is cDNA.

20. A vector comprising one or more of the nucleic acids of any one of claims 8-19.

21. A vector comprising two of the nucleic acids of any one of claims 8-19, wherein the vector encodes the VH region and the VL region that together bind to CCR8.

22. A pair of vectors, wherein each vector comprises one of the nucleic acids of any one of claims 8-19, wherein together the pair of vectors encodes the VH region and the VL region that together bind to CCR8.

23. A cell comprising the vector of claim 20 or 21, or the pair of vectors of claim 22.

24. The cell of claim 23, wherein the cell is a CHO cell.

25. A cell comprising one or more of the nucleic acids of any one of claims 8-19.

26. A cell comprising two of the nucleic acids of any one of claims 8-19.

27. The cell of claim 26, wherein the two nucleic acids together encode the VH region and the VL region that together bind to CCR8.

28. A method of producing an antibody or an antigen-binding fragment thereof, the method comprising:
(a) culturing the cell of any one of claims 23-27 under conditions sufficient for the cell to produce the antibody or the antigen-binding fragment; and
(b) collecting the antibody or the antigen-binding fragment produced by the cell.

29. An antibody or antigen-binding fragment thereof that binds to CCR8 comprising a heavy chain variable region (VH) comprising an amino acid sequence that is at least 80% identical to a selected VH sequence, and a light chain variable region (VL) comprising an amino acid sequence that is at least 80% identical to a selected VL sequence,
wherein the selected VH sequence is selected from the group consisting of: SEQ ID NOs: 37, 38, 39, 63, 64, 65, 69, 70, 71, 216, 261, 262, 263, 264, 265, 266, and 267,
wherein the selected VL sequence is selected from the group consisting of: SEQ ID NOs: 40, 41, 42, 66, 67, and 68.

30. The antibody or antigen-binding fragment thereof of claim 29, wherein the VH sequence and the VL sequence are one of the following:
(1) the VH comprises the sequence of SEQ ID NO: 37 and the VL comprises the sequence of SEQ ID NO: 40;
(2) the VH comprises the sequence of SEQ ID NO: 38 and the VL comprises the sequence of SEQ ID NO: 41;
(3) the VH comprises the sequence of SEQ ID NO: 39 and the VL comprises the sequence of SEQ ID NO: 42;
(4) the VH comprises the sequence of SEQ ID NO: 37 and the VL comprises the sequence of SEQ ID NO: 41;
(5) the VH comprises the sequence of SEQ ID NO: 37 and the VL comprises the sequence of SEQ ID NO: 42;
(6) the VH comprises the sequence of SEQ ID NO: 38 and the VL comprises the sequence of SEQ ID NO: 40;
(7) the VH comprises the sequence of SEQ ID NO: 38 and the VL comprises the sequence of SEQ ID NO: 42;
(8) the VH comprises the sequence of SEQ ID NO: 39 and the VL comprises the sequence of SEQ ID NO: 40;
(9) the VH comprises the sequence of SEQ ID NO: 39 and the VL comprises the sequence of SEQ ID NO: 41;
(10) the VH comprises the sequence of SEQ ID NO: 37 and the VL comprises the sequence of SEQ ID NO: 68;
(11) the VH comprises the sequence of SEQ ID NO: 63 and the VL comprises the sequence of SEQ ID NO: 68;
(12) the VH comprises the sequence of SEQ ID NO: 63 and the VL comprises the sequence of SEQ ID NO: 66;
(13) the VH comprises the sequence of SEQ ID NO: 65 and the VL comprises the sequence of SEQ ID NO: 68;
(14) the VH comprises the sequence of SEQ ID NO: 64 and the VL comprises the sequence of SEQ ID NO: 67;
(15) the VH comprises the sequence of SEQ ID NO: 63 and the VL comprises the sequence of SEQ ID NO: 42;
(16) the VH comprises the sequence of SEQ ID NO: 216 and the VL comprises the sequence of SEQ ID NO: 42;
(17) the VH comprises the sequence of SEQ ID NO: 261 and the VL comprises the sequence of SEQ ID NO: 41;
(18) the VH comprises the sequence of SEQ ID NO: 262 and the VL comprises the sequence of SEQ ID NO: 41;
(19) the VH comprises the sequence of SEQ ID NO: 263 and the VL comprises the sequence of SEQ ID NO: 41;
(20) the VH comprises the sequence of SEQ ID NO: 264 and the VL comprises the sequence of SEQ ID NO: 41;
(21) the VH comprises the sequence of SEQ ID NO: 265 and the VL comprises the sequence of SEQ ID NO: 41;
(22) the VH comprises the sequence of SEQ ID NO: 266 and the VL comprises the sequence of SEQ ID NO: 41; and
(23) the VH comprises the sequence of SEQ ID NO: 267 and the VL comprises the sequence of SEQ ID NO: 41.

31. The antibody or antigen-binding fragment thereof of claim 29 or 30, wherein the antibody or antigen-binding fragment specifically binds to human CCR8, mouse CCR8 or monkey CCR8.

32. The antibody or antigen-binding fragment thereof of any one of claims 29-31, wherein the antibody or antigen-binding fragment is a human or humanized antibody or antigen-binding fragment thereof.

33. The antibody or antigen-binding fragment thereof of any one of claims 29-32, wherein the antibody or antigen-binding fragment is a single-chain variable fragment (scFv), a one-armed antibody, or a multi-specific antibody (e.g., a bispecific antibody).

34. An antibody or antigen-binding fragment thereof that binds to CCR8 comprising a heavy chain variable region (VH) comprising VH CDRs 1, 2, 3, and a light chain variable region comprising VL CDRs 1, 2, 3, wherein the VH CDRs 1, 2, 3 and the VL CDRs 1, 2, 3 are identical to complementarity determining regions in the antibody or antigen-binding fragment thereof of any one of claims 1-7 and 29-33.

35. The antibody or antigen-binding fragment thereof of claim 34, wherein the VH CDRs 1, 2, 3 are identical to complementarity determining regions in SEQ ID NO: 37, 38, 39, 63, 64, 65, 69, 70, 71, 216, 261, 262, 263, 264, 265, 266, or 267, and the VL CDRs 1, 2, 3 are identical to complementary determining regions in SEQ ID NO: 40, 41, 42, 66, 67, or 68.

36. An antibody or antigen-binding fragment thereof that cross-competes with the antibody or antigen-binding fragment thereof of any one of claims 1-7 and 29-35.

37. An antibody-drug conjugate comprising the antibody or antigen-binding fragment thereof of any one of claims 1-7 and 29-36 covalently bound to a therapeutic agent.

38. The antibody drug conjugate of claim 37, wherein the therapeutic agent is a cytotoxic or cytostatic agent.

39. A method of treating a subject having cancer, the method comprising administering a therapeutically effective amount of a composition comprising the antibody or antigen-binding fragment thereof of any one of claims 1-7 and 29-36, or the antibody-drug conjugate of claim 37 or 38, to the subject.

40. The method of claim 39, wherein the subject has a solid tumor.

41. The method of claim 39 or 40, wherein the cancer is breast cancer, melanoma, non-small cell lung cancer, lung adenocarcinoma, colon cancer, or colorectal cancer (e.g., colorectal adenocarcinoma).

42. The method of any one of claims 39-41, wherein the cancer is resistant to a PD-1 pathway inhibitor treatment.

43. The method of any one of claims 39-42, wherein the method further comprises administering a PD-1 pathway inhibitor to the subject.

44. The method of claim 43, wherein the PD-1 pathway inhibitor is an anti-PD-1 antibody (e.g., Pembrolizumab, Nivolumab, or Cemiplimab).

45. The method of claim 43, wherein the PD-1 pathway inhibitor is an anti-PD-L1 antibody (e.g., Atezolizumab, Avelumab, or Durvalumab).

46. A method of decreasing the rate of tumor growth, the method comprising contacting a tumor cell with an effective amount of a composition comprising an antibody or antigen-binding fragment thereof of any one of claims 1-7 and 29-36, or the antibody-drug conjugate of claim 37 or 38.

47. A method of killing a tumor cell, the method comprising contacting a tumor cell with an effective amount of a composition comprising the antibody or antigen-binding fragment thereof of any one of claims 1-7 and 29-36, or the antibody-drug conjugate of claim 37 or 38.

48. A pharmaceutical composition comprising the antibody or antigen-binding fragment thereof of any one of claims 1-7 and 29-36, and a pharmaceutically acceptable carrier.

49. A pharmaceutical composition comprising the antibody drug conjugate of claim 37 or 38, and a pharmaceutically acceptable carrier.


SEQUENCE LISTING (267 sequences)

SEQ ID NO: 1 - protein, 5 aa, synthetic construct
SYVMH

SEQ ID NO: 2 - protein, 17 aa, synthetic construct
VISYDGSDKYYADSVKG

SEQ ID NO: 3 - protein, 16 aa, synthetic construct
GRGYRYGQSYYYGMDV

SEQ ID NO: 4 - protein, 5 aa, synthetic construct
SYVVH

SEQ ID NO: 5 - protein, 17 aa, synthetic construct
VISYDGDNKFYADSVKG

SEQ ID NO: 6 - protein, 12 aa, synthetic construct
GRNYYYYYGLDV

SEQ ID NO: 7 - protein, 5 aa, synthetic construct
TYVMH

SEQ ID NO: 8 - protein, 17 aa, synthetic construct
VISYDGNNKYYADSVKG

SEQ ID NO: 9 - protein, 12 aa, synthetic construct
GRSYVNYYGLDV

SEQ ID NO: 10 - protein, 7 aa, synthetic construct
GFSFSSY

SEQ ID NO: 11 - protein, 6 aa, synthetic construct
SYDGSD

SEQ ID NO: 12 - protein, 16 aa, synthetic construct
GRGYRYGQSYYYGMDV

SEQ ID NO: 13 - protein, 7 aa, synthetic construct
GFTFSSY

SEQ ID NO: 14 - protein, 6 aa, synthetic construct
SYDGDN

SEQ ID NO: 15 - protein, 12 aa, synthetic construct
GRNYYYYYGLDV

SEQ ID NO: 16 - protein, 7 aa, synthetic construct
GFTFSTY

SEQ ID NO: 17 - protein, 6 aa, synthetic construct
SYDGNN

SEQ ID NO: 18 - protein, 12 aa, synthetic construct
GRSYVNYYGLDV

SEQ ID NO: 19 - protein, 16 aa, synthetic construct
KSSQSLLYSDGKTYLY

SEQ ID NO: 20 - protein, 7 aa, synthetic construct
EVSNRFS

SEQ ID NO: 21 - protein, 9 aa, synthetic construct
MQSIKLPLT

SEQ ID NO: 22 - protein, 16 aa, synthetic construct
KSSQSLLYSDGKTYLY

SEQ ID NO: 23 - protein, 7 aa, synthetic construct
EVSNRFS

SEQ ID NO: 24 - protein, 9 aa, synthetic construct
MQSLKVPPT

SEQ ID NO: 25 - protein, 16 aa, synthetic construct
KSSQSLLYSDGKTYLY

SEQ ID NO: 26 - protein, 7 aa, synthetic construct
EVSNRFS

SEQ ID NO: 27 - protein, 9 aa, synthetic construct
MQSVKIPLT

SEQ ID NO: 28 - protein, 16 aa, synthetic construct
KSSQSLLYSDGKTYLY

SEQ ID NO: 29 - protein, 7 aa, synthetic construct
EVSNRFS

SEQ ID NO: 30 - protein, 9 aa, synthetic construct
MQSIKLPLT

SEQ ID NO: 31 - protein, 16 aa, synthetic construct
KSSQSLLYSDGKTYLY

SEQ ID NO: 32 - protein, 7 aa, synthetic construct
EVSNRFS

SEQ ID NO: 33 - protein, 9 aa, synthetic construct
MQSLKVPPT

SEQ ID NO: 34 - protein, 16 aa, synthetic construct
KSSQSLLYSDGKTYLY

SEQ ID NO: 35 - protein, 7 aa, synthetic construct
EVSNRFS

SEQ ID NO: 36 - protein, 9 aa, synthetic construct
MQSVKIPLT

SEQ ID NO: 37 - protein, 125 aa, synthetic construct
QVQLVESGGGVVQPGRSLRLSCAASGFSFSSYVMHWVRQAPGKGLEWVAVISYDGSDKYYADSVKGRFTISRDNSKNTLFLQMNSLRPEDTAVYYCARGRGYRYGQSYYYGMDVWGQGTTVTVSS

SEQ ID NO: 38 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRNYYYYYGLDVWGQGTTVTVSS

SEQ ID NO: 39 - protein, 121 aa, synthetic construct
QVQLVESGGGVVQPGRSLRLSCAASGFTFSTYVMHWVRQAPGKGLEWVAVISYDGNNKYYADSVKGRFIISRDNSKNTLFLQMNSLRGEDTAVYYCARGRSYVNYYGLDVWGQGTTVTVSS

SEQ ID NO: 40 - protein, 112 aa, synthetic construct
DIVMTQTPLSLSVTPGQPASISCKSSQSLLYSDGKTYLYWYLQKPGQPPQILIFEVSNRFSGVPDRFSGSGSGTDFTLKISRVEAEDVGVYYCMQSIKLPLTFGGGTKVEIK

SEQ ID NO: 41 - protein, 112 aa, synthetic construct
DIVMTQTPLSLSVTPGQPASISCKSSQSLLYSDGKTYLYWYLQKAGQPPQLLIYEVSNRFSGVPDRFSGSGSGTDFTLKISRVEAEDVGVFYCMQSLKVPPTFGQGTKVDIK

SEQ ID NO: 42 - protein, 112 aa, synthetic construct
DIVMTQTPLSLSVTPGQPASISCKSSQSLLYSDGKTYLYWYLQKPGQPPHLLIYEVSNRFSGVPKRFSGSGSGTDFTLKISRVEAEDVGVYYCMQSVKIPLTFGGGTKVEIK

SEQ ID NO: 43 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 44 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 45 - protein, 355 aa, Macaca mulatta
MDYTLDPSMTTMTDYYYPDSLSSPCDGELIQRNDKLLLAVFYCLLFVFSLLGNSLVILVLVVCKKLRNITDIYLLNLALSDLLFVFSFPFQTYYQLDQWVFGTVMCKVVSGFYYIGFYSSMFFITLMSVDRYLAVVHAVYAIKVRTIRMGTTLSLVVWLTAIMATIPLLVFYQVASEDGVLQCYSFYNQQTLKWKIFTNFEMNILGLLIPFTIFMFCYIKILHQLKRCQNHNKTKAIRLVLIVVIASLLFWVPFNVVLFLTSLHSMHILDGCSISQQLNYATHVTEIISFTHCCVNPVIYAFVGEKFKKHLSEIFQKSCSHIFIYLGRQMPRESCEKSSSCQQHSFRSSSIDYIL

SEQ ID NO: 46 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 47 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 48 - protein, 352 aa, Homo sapiens [sequence omitted]

SEQ ID NO: 49 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 50 - protein, 117 aa, synthetic construct
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSS

SEQ ID NO: 51 - protein, 112 aa, synthetic construct
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIK

SEQ ID NO: 52 - protein, 119 aa, synthetic construct
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 53 - protein, 112 aa, synthetic construct
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 54 - protein, 122 aa, synthetic construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 55 - protein, 112 aa, synthetic construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 56 - protein, 369 aa, synthetic construct
DVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGSIFSSTAMAWYRQAPGKRRDLVAAISTVGVTKYADSVKGRFTISRDNSKNTVYLQMNSLRPEDTAVYYCTSDPRRGWDTRYWGQGTLVTVSS

SEQ ID NO: 57 - protein, 125 aa, synthetic construct
EVQLVESGGALVKPGGSLRLSCAASGFTFSTYALYWVRQAPGKGLEWVGRIRSKSNNYATYYADSVKDRFTISRDDSKNTLYLQMNSLKTEDTAVYYCTRARFYYSDYGYAMDYWGQGTLVTVSS

SEQ ID NO: 58 - protein, 114 aa, synthetic construct
DIVMTQSPDSLAVSLGERATINCRSSKSLLHSNRNTYLYWYQQKPGQPPKLLIYRMSQLASGVPDRFSGSGSGTDFTLTISSLQAEDVAVYYCMQHLEYPLTFGQGTKLEIKRT

SEQ ID NO: 59 - protein, 121 aa, synthetic construct
EVQLVESGGGLVQPGGSLKLSCAASGFTFNTYAMNWVRQASGKGLEWVGRIRSKSNNYATYYADSVKDRFTISRDDSKNTAYLQMNSLKTEDTAVYYCVRGLLRYRFFDVWGQGTTVTVSS

SEQ ID NO: 60 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 61 - protein, 4 aa, synthetic construct
GGGS

SEQ ID NO: 62 - protein, 8 aa, synthetic construct
GGGSGGGS

SEQ ID NO: 63 - protein, 125 aa, synthetic construct
QVQLVESGGGVVQPGRSLRLSCAASGFSFSSYVMHWVRQAPGKGLEWVAVISYSGSDKYYADSVKGRFTISRDNSKNTLFLQMNSLRPEDTAVYYCARGRGYRYGQSYYYGMDVWGQGTTVTVSS

SEQ ID NO: 64 - protein, 121 aa, synthetic construct
EVQLVESGGGVVQPGRSLRLSCAASGFTFSSYVVHWVRQAPGKGLEWVAVISYSGDNKFYADSVKGRFTISRDNSKNTLYLQMNSLRTEDTAVYYCARGRNYYYYYGLDVWGQGTTVTVSS

SEQ ID NO: 65 - protein, 121 aa, synthetic construct
QVQLVESGGGVVQPGRSLRLSCAASGFTFSTYVMHWVRQAPGKGLEWVAVISYSGNNKYYADSVKGRFIISRDNSKNTLFLQMNSLRGEDTAVYYCARGRSYVNYYGLDVWGQGTTVTVSS

SEQ ID NO: 66 - protein, 112 aa, synthetic construct
DIVMTQTPLSLSVTPGQPASISCKSSQSLLYSSGKTYLYWYLQKPGQPPQILIFEVSNRFSGVPDRFSGSGSGTDFTLKISRVEAEDVGVYYCMQSIKLPLTFGGGTKVEIK

SEQ ID NO: 67 - protein, 112 aa, synthetic construct
DIVMTQTPLSLSVTPGQPASISCKSSQSLLYSSGKTYLYWYLQKAGQPPQLLIYEVSNRFSGVPDRFSGSGSGTDFTLKISRVEAEDVGVFYCMQSLKVPPTFGQGTKVDIK

SEQ ID NO: 68 - protein, 112 aa, synthetic construct
DIVMTQTPLSLSVTPGQPASISCKSSQSLLYSSGKTYLYWYLQKPGQPPHLLIYEVSNRFSGVPKRFSGSGSGTDFTLKISRVEAEDVGVYYCMQSVKIPLTFGGGTKVEIK

SEQ ID NO: 69 - protein, 125 aa, synthetic construct
QVQLVESGGGVVQPGRSLRLSCAASGFSFSSYVMHWVRQAPGKGLEWVAVISYDGSDKYYADSVKGRFTISRDNAKNTLFLQMNSLRPEDTAVYYCARGRGYRYGQSYYYGMDVWGQGTTVTVSS

SEQ ID NO: 70 - protein, 121 aa, synthetic construct
EVQLVESGGGVVQPGRSLRLSCAASGFTFSSYVVHWVRQAPGKGLEWVAVISYDGDNKFYADSVKGRFTISRDNAKNTLYLQMNSLRTEDTAVYYCARGRNYYYYYGLDVWGQGTTVTVSS

SEQ ID NO: 71 - protein, 121 aa, synthetic construct
QVQLVESGGGVVQPGRSLRLSCAASGFTFSTYVMHWVRQAPGKGLEWVAVISYDGNNKYYADSVKGRFIISRDNAKNTLFLQMNSLRGEDTAVYYCARGRSYVNYYGLDVWGQGTTVTVSS

SEQ ID NO: 72 - protein, 5 aa, synthetic construct
SYVMH

SEQ ID NO: 73 - protein, 17 aa, synthetic construct
VISYSGSDKYYADSVKG

SEQ ID NO: 74 - protein, 16 aa, synthetic construct
GRGYRYGQSYYYGMDV

SEQ ID NO: 75 - protein, 5 aa, synthetic construct
SYVVH

SEQ ID NO: 76 - protein, 17 aa, synthetic construct
VISYSGDNKFYADSVKG

SEQ ID NO: 77 - protein, 12 aa, synthetic construct
GRNYYYYYGLDV

SEQ ID NO: 78 - protein, 5 aa, synthetic construct
TYVMH

SEQ ID NO: 79 - protein, 17 aa, synthetic construct
VISYSGNNKYYADSVKG

SEQ ID NO: 80 - protein, 12 aa, synthetic construct
GRSYVNYYGLDV

SEQ ID NO: 81 - protein, 5 aa, synthetic construct
SYVMH

SEQ ID NO: 82 - protein, 17 aa, synthetic construct
VISYAGSDKYYADSVKG

SEQ ID NO: 83 - protein, 16 aa, synthetic construct
GRGYRYGQSYYYGMDV

SEQ ID NO: 84 - protein, 5 aa, synthetic construct
SYVVH

SEQ ID NO: 85 - protein, 17 aa, synthetic construct
VISYAGDNKFYADSVKG

SEQ ID NO: 86 - protein, 12 aa, synthetic construct
GRNYYYYYGLDV

SEQ ID NO: 87 - protein, 5 aa, synthetic construct
TYVMH

SEQ ID NO: 88 - protein, 17 aa, synthetic construct
VISYAGNNKYYADSVKG

SEQ ID NO: 89 - protein, 12 aa, synthetic construct
GRSYVNYYGLDV

SEQ ID NO: 90 - protein, 5 aa, synthetic construct
SYVMH

SEQ ID NO: 91 - protein, 17 aa, synthetic construct
VISYDASDKYYADSVKG

SEQ ID NO: 92 - protein, 16 aa, synthetic construct
GRGYRYGQSYYYGMDV

SEQ ID NO: 93 - protein, 5 aa, synthetic construct
SYVVH

SEQ ID NO: 94 - protein, 17 aa, synthetic construct
VISYDADNKFYADSVKG

SEQ ID NO: 95 - protein, 12 aa, synthetic construct
GRNYYYYYGLDV

SEQ ID NO: 96 - protein, 5 aa, synthetic construct
TYVMH

SEQ ID NO: 97 - protein, 17 aa, synthetic construct
VISYDANNKYYADSVKG

SEQ ID NO: 98 - protein, 12 aa, synthetic construct
GRSYVNYYGLDV

SEQ ID NO: 99 - protein, 5 aa, synthetic construct
SYVMH

SEQ ID NO: 100 - protein, 17 aa, synthetic construct
  VARIANT 5: note = D, S, A
  VARIANT 6: note = G, A
VISYXXSDKYYADSVKG

SEQ ID NO: 101 - protein, 16 aa, synthetic construct
GRGYRYGQSYYYGMDV

SEQ ID NO: 102 - protein, 5 aa, synthetic construct
SYVVH

SEQ ID NO: 103 - protein, 17 aa, synthetic construct
  VARIANT 5: note = D, S, A
  VARIANT 6: note = G, A
VISYXXDNKFYADSVKG

SEQ ID NO: 104 - protein, 12 aa, synthetic construct
  VARIANT 3: note = N, S
  VARIANT 5: note = Y, V
  VARIANT 6: note = Y, N
GRXYXXYYGLDV

SEQ ID NO: 105 - protein, 5 aa, synthetic construct
TYVMH

SEQ ID NO: 106 - protein, 17 aa, synthetic construct
  VARIANT 5: note = D, S, A
  VARIANT 6: note = G, A
VISYXXNNKYYADSVKG

SEQ ID NO: 107 - protein, 12 aa, synthetic construct
GRSYVNYYGLDV

SEQ ID NO: 108 - protein, 16 aa, synthetic construct
KSSQSLLYSSGKTYLY

SEQ ID NO: 109 - protein, 7 aa, synthetic construct
EVSNRFS

SEQ ID NO: 110 - protein, 9 aa, synthetic construct
MQSIKLPLT

SEQ ID NO: 111 - protein, 16 aa, synthetic construct
KSSQSLLYSSGKTYLY

SEQ ID NO: 112 - protein, 7 aa, synthetic construct
EVSNRFS

SEQ ID NO: 113 - protein, 9 aa, synthetic construct
MQSLKVPPT

SEQ ID NO: 114 - protein, 16 aa, synthetic construct
KSSQSLLYSSGKTYLY

SEQ ID NO: 115 - protein, 7 aa, synthetic construct
EVSNRFS

SEQ ID NO: 116 - protein, 9 aa, synthetic construct
MQSVKIPLT

SEQ ID NO: 117 - protein, 16 aa, synthetic construct
KSSQSLLYSAGKTYLY

SEQ ID NO: 118 - protein, 7 aa, synthetic construct
EVSNRFS

SEQ ID NO: 119 - protein, 9 aa, synthetic construct
MQSIKLPLT

SEQ ID NO: 120 - protein, 16 aa, synthetic construct
KSSQSLLYSAGKTYLY

SEQ ID NO: 121 - protein, 7 aa, synthetic construct
EVSNRFS

SEQ ID NO: 122 - protein, 9 aa, synthetic construct
MQSLKVPPT

SEQ ID NO: 123 - protein, 16 aa, synthetic construct
KSSQSLLYSAGKTYLY

SEQ ID NO: 124 - protein, 7 aa, synthetic construct
EVSNRFS

SEQ ID NO: 125 - protein, 9 aa, synthetic construct
MQSVKIPLT

SEQ ID NO: 126 - protein, 16 aa, synthetic construct
KSSQSLLYSDAKTYLY

SEQ ID NO: 127 - protein, 7 aa, synthetic construct
EVSNRFS

SEQ ID NO: 128 - protein, 9 aa, synthetic construct
MQSIKLPLT

SEQ ID NO: 129 - protein, 16 aa, synthetic construct
KSSQSLLYSDAKTYLY

SEQ ID NO: 130 - protein, 7 aa, synthetic construct
EVSNRFS

SEQ ID NO: 131 - protein, 9 aa, synthetic construct
MQSLKVPPT

SEQ ID NO: 132 - protein, 16 aa, synthetic construct
KSSQSLLYSDAKTYLY

SEQ ID NO: 133 - protein, 7 aa, synthetic construct
EVSNRFS

SEQ ID NO: 134 - protein, 9 aa, synthetic construct
MQSVKIPLT

SEQ ID NO: 135 - protein, 16 aa, synthetic construct
  VARIANT 10: note = D, S, A
  VARIANT 11: note = G, A
KSSQSLLYSXXKTYLY

SEQ ID NO: 136 - protein, 7 aa, synthetic construct
EVSNRFS

SEQ ID NO: 137 - protein, 9 aa, synthetic construct
MQSIKLPLT

SEQ ID NO: 138 - protein, 16 aa, synthetic construct
  VARIANT 10: note = D, S, A
  VARIANT 11: note = G, A
KSSQSLLYSXXKTYLY

SEQ ID NO: 139 - protein, 7 aa, synthetic construct
EVSNRFS

SEQ ID NO: 140 - protein, 9 aa, synthetic construct
MQSLKVPPT

SEQ ID NO: 141 - protein, 16 aa, synthetic construct
  VARIANT 10: note = D, S, A
  VARIANT 11: note = G, A
KSSQSLLYSXXKTYLY

SEQ ID NO: 142 - protein, 7 aa, synthetic construct
EVSNRFS

SEQ ID NO: 143 - protein, 9 aa, synthetic construct
MQSVKIPLT

SEQ ID NO: 144 - protein, 7 aa, synthetic construct
GFSFSSY

SEQ ID NO: 145 - protein, 6 aa, synthetic construct
SYSGSD

SEQ ID NO: 146 - protein, 16 aa, synthetic construct
GRGYRYGQSYYYGMDV

SEQ ID NO: 147 - protein, 7 aa, synthetic construct
GFTFSSY

SEQ ID NO: 148 - protein, 6 aa, synthetic construct
SYSGDN

SEQ ID NO: 149 - protein, 12 aa, synthetic construct
GRNYYYYYGLDV

SEQ ID NO: 150 - protein, 7 aa, synthetic construct
GFTFSTY

SEQ ID NO: 151 - protein, 6 aa, synthetic construct
SYSGNN

SEQ ID NO: 152 - protein, 12 aa, synthetic construct
GRSYVNYYGLDV

SEQ ID NO: 153 - protein, 7 aa, synthetic construct
GFSFSSY

SEQ ID NO: 154 - protein, 6 aa, synthetic construct
SYAGSD

SEQ ID NO: 155 - protein, 16 aa, synthetic construct
GRGYRYGQSYYYGMDV

SEQ ID NO: 156 - protein, 7 aa, synthetic construct
GFTFSSY

SEQ ID NO: 157 - protein, 6 aa, synthetic construct
SYAGDN

SEQ ID NO: 158 - protein, 12 aa, synthetic construct
GRNYYYYYGLDV

SEQ ID NO: 159 - protein, 7 aa, synthetic construct
GFTFSTY

SEQ ID NO: 160 - protein, 6 aa, synthetic construct
SYAGNN

SEQ ID NO: 161 - protein, 12 aa, synthetic construct
GRSYVNYYGLDV

SEQ ID NO: 162 - protein, 7 aa, synthetic construct
GFSFSSY

SEQ ID NO: 163 - protein, 6 aa, synthetic construct
SYDASD

SEQ ID NO: 164 - protein, 16 aa, synthetic construct
GRGYRYGQSYYYGMDV

SEQ ID NO: 165 - protein, 7 aa, synthetic construct
GFTFSSY

SEQ ID NO: 166 - protein, 6 aa, synthetic construct
SYDADN

SEQ ID NO: 167 - protein, 12 aa, synthetic construct
GRNYYYYYGLDV

SEQ ID NO: 168 - protein, 7 aa, synthetic construct
GFTFSTY

SEQ ID NO: 169 - protein, 6 aa, synthetic construct
SYDANN

SEQ ID NO: 170 - protein, 12 aa, synthetic construct
GRSYVNYYGLDV

SEQ ID NO: 171 - protein, 7 aa, synthetic construct
GFSFSSY

SEQ ID NO: 172 - protein, 6 aa, synthetic construct
  VARIANT 3: note = D, S, A
  VARIANT 4: note = G, A
SYXXSD

SEQ ID NO: 173 - protein, 16 aa, synthetic construct
GRGYRYGQSYYYGMDV

SEQ ID NO: 174 - protein, 7 aa, synthetic construct
GFTFSSY

SEQ ID NO: 175 - protein, 6 aa, synthetic construct
  VARIANT 3: note = D, S, A
  VARIANT 4: note = G, A
SYXXDN

SEQ ID NO: 176 - protein, 12 aa, synthetic construct
  VARIANT 3: note = N, S
  VARIANT 5: note = Y, V
  VARIANT 6: note = Y, N
GRXYXXYYGLDV

SEQ ID NO: 177 - protein, 7 aa, synthetic construct
GFTFSTY

SEQ ID NO: 178 - protein, 6 aa, synthetic construct
  VARIANT 3: note = D, S, A
  VARIANT 4: note = G, A
SYXXNN

SEQ ID NO: 179 - protein, 12 aa, synthetic construct
GRSYVNYYGLDV

SEQ ID NO: 180 - protein, 16 aa, synthetic construct
KSSQSLLYSSGKTYLY

SEQ ID NO: 181 - protein, 7 aa, synthetic construct
EVSNRFS

SEQ ID NO: 182 - protein, 9 aa, synthetic construct
MQSIKLPLT

SEQ ID NO: 183 - protein, 16 aa, synthetic construct
KSSQSLLYSSGKTYLY

SEQ ID NO: 184 - protein, 7 aa, synthetic construct
EVSNRFS

SEQ ID NO: 185 - protein, 9 aa, synthetic construct
MQSLKVPPT

SEQ ID NO: 186 - protein, 16 aa, synthetic construct
KSSQSLLYSSGKTYLY

SEQ ID NO: 187 - protein, 7 aa, synthetic construct
EVSNRFS

SEQ ID NO: 188 - protein, 9 aa, synthetic construct
MQSVKIPLT

SEQ ID NO: 189 - protein, 16 aa, synthetic construct
KSSQSLLYSAGKTYLY

SEQ ID NO: 190 - protein, 7 aa, synthetic construct
EVSNRFS

SEQ ID NO: 191 - protein, 9 aa, synthetic construct
MQSIKLPLT

SEQ ID NO: 192 - protein, 16 aa, synthetic construct
KSSQSLLYSAGKTYLY

SEQ ID NO: 193 - protein, 7 aa, synthetic construct
EVSNRFS

SEQ ID NO: 194 - protein, 9 aa, synthetic construct
MQSLKVPPT

SEQ ID NO: 195 - protein, 16 aa, synthetic construct
KSSQSLLYSAGKTYLY

SEQ ID NO: 196 - protein, 7 aa, synthetic construct
EVSNRFS

SEQ ID NO: 197 - protein, 9 aa, synthetic construct
MQSVKIPLT

SEQ ID NO: 198 - protein, 16 aa, synthetic construct
KSSQSLLYSDAKTYLY

SEQ ID NO: 199 - protein, 7 aa, synthetic construct
EVSNRFS

SEQ ID NO: 200 - protein, 9 aa, synthetic construct
MQSIKLPLT

SEQ ID NO: 201 - protein, 16 aa, synthetic construct
KSSQSLLYSDAKTYLY

SEQ ID NO: 202 - protein, 7 aa, synthetic construct
EVSNRFS

SEQ ID NO: 203 - protein, 9 aa, synthetic construct
MQSLKVPPT

SEQ ID NO: 204 - protein, 16 aa, synthetic construct
KSSQSLLYSDAKTYLY

SEQ ID NO: 205 - protein, 7 aa, synthetic construct
EVSNRFS

SEQ ID NO: 206 - protein, 9 aa, synthetic construct
MQSVKIPLT

SEQ ID NO: 207 - protein, 16 aa, synthetic construct
  VARIANT 10: note = D, S, A
  VARIANT 11: note = G, A
KSSQSLLYSXXKTYLY

SEQ ID NO: 208 - protein, 7 aa, synthetic construct
EVSNRFS

SEQ ID NO: 209 - protein, 9 aa, synthetic construct
MQSIKLPLT

SEQ ID NO: 210 - protein, 16 aa, synthetic construct
  VARIANT 10: note = D, S, A
  VARIANT 11: note = G, A
KSSQSLLYSXXKTYLY

SEQ ID NO: 211 - protein, 7 aa, synthetic construct
EVSNRFS

SEQ ID NO: 212 - protein, 9 aa, synthetic construct
MQSLKVPPT

SEQ ID NO: 213 - protein, 16 aa, synthetic construct
  VARIANT 10: note = D, S, A
  VARIANT 11: note = G, A
KSSQSLLYSXXKTYLY

SEQ ID NO: 214 - protein, 7 aa, synthetic construct
EVSNRFS

SEQ ID NO: 215 - protein, 9 aa, synthetic construct
MQSVKIPLT

SEQ ID NO: 216 - protein, 125 aa, synthetic construct
QVQLVESGGGVVQPGRSLRLSCAASGFSFSSYVMHWVRQAPGKGLEWVAVISYSGSDKYYADSVKGRFTISRDNAKNTLFLQMNSLRPEDTAVYYCARGRGYRYGQSYYYGMDVWGQGTTVTVSS

SEQ ID NO: 217 - protein, 120 aa, synthetic construct [sequence omitted]

SEQ ID NO: 218 - protein, 111 aa, synthetic construct [sequence omitted]

SEQ ID NO: 219 - protein, 5 aa, synthetic construct
SYVVH

SEQ ID NO: 220 - protein, 17 aa, synthetic construct
VISYDGDNKFYADSVKG

SEQ ID NO: 221 - protein, 12 aa, synthetic construct
GRSYYYYYGLDV

SEQ ID NO: 222 - protein, 5 aa, synthetic construct
SYVVH

SEQ ID NO: 223 - protein, 17 aa, synthetic construct
VISYDGDNKFYADSVKG

SEQ ID NO: 224 - protein, 12 aa, synthetic construct
GRNYVYYYGLDV

SEQ ID NO: 225 - protein, 5 aa, synthetic construct
SYVVH

SEQ ID NO: 226 - protein, 17 aa, synthetic construct
VISYDGDNKFYADSVKG

SEQ ID NO: 227 - protein, 12 aa, synthetic construct
GRNYYNYYGLDV

SEQ ID NO: 228 - protein, 5 aa, synthetic construct
SYVVH

SEQ ID NO: 229 - protein, 17 aa, synthetic construct
VISYDGDNKFYADSVKG

SEQ ID NO: 230 - protein, 12 aa, synthetic construct
GRSYVYYYGLDV

SEQ ID NO: 231 - protein, 5 aa, synthetic construct
SYVVH

SEQ ID NO: 232 - protein, 17 aa, synthetic construct
VISYDGDNKFYADSVKG

SEQ ID NO: 233 - protein, 12 aa, synthetic construct
GRSYYNYYGLDV

SEQ ID NO: 234 - protein, 5 aa, synthetic construct
SYVVH

SEQ ID NO: 235 - protein, 17 aa, synthetic construct
VISYDGDNKFYADSVKG

SEQ ID NO: 236 - protein, 12 aa, synthetic construct
GRNYVNYYGLDV

SEQ ID NO: 237 - protein, 5 aa, synthetic construct
SYVVH

SEQ ID NO: 238 - protein, 17 aa, synthetic construct
VISYDGDNKFYADSVKG

SEQ ID NO: 239 - protein, 12 aa, synthetic construct
GRSYVNYYGLDV

SEQ ID NO: 240 - protein, 7 aa, synthetic construct
GFTFSSY

SEQ ID NO: 241 - protein, 6 aa, synthetic construct
SYDGDN

SEQ ID NO: 242 - protein, 12 aa, synthetic construct
GRSYYYYYGLDV

SEQ ID NO: 243 - protein, 7 aa, synthetic construct
GFTFSSY

SEQ ID NO: 244 - protein, 6 aa, synthetic construct
SYDGDN

SEQ ID NO: 245 - protein, 12 aa, synthetic construct
GRNYVYYYGLDV

SEQ ID NO: 246 - protein, 7 aa, synthetic construct
GFTFSSY

SEQ ID NO: 247 - protein, 6 aa, synthetic construct
SYDGDN

SEQ ID NO: 248 - protein, 12 aa, synthetic construct
GRNYYNYYGLDV

SEQ ID NO: 249 - protein, 7 aa, synthetic construct
GFTFSSY

SEQ ID NO: 250 - protein, 6 aa, synthetic construct
SYDGDN

SEQ ID NO: 251 - protein, 12 aa, synthetic construct
GRSYVYYYGLDV

SEQ ID NO: 252 - protein, 7 aa, synthetic construct
GFTFSSY

SEQ ID NO: 253 - protein, 6 aa, synthetic construct
SYDGDN

SEQ ID NO: 254 - protein, 12 aa, synthetic construct
GRSYYNYYGLDV

SEQ ID NO: 255 - protein, 7 aa, synthetic construct
GFTFSSY

SEQ ID NO: 256 - protein, 6 aa, synthetic construct
SYDGDN

SEQ ID NO: 257 - protein, 12 aa, synthetic construct
GRNYVNYYGLDV

SEQ ID NO: 258 - protein, 7 aa, synthetic construct
GFTFSSY

SEQ ID NO: 259 - protein, 6 aa, synthetic construct
SYDGDN

SEQ ID NO: 260 - protein, 12 aa, synthetic construct
GRSYVNYYGLDV

SEQ ID NO: 261 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRSYYYYYGLDVWGQGTTVTVSS

SEQ ID NO: 262 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRNYVYYYGLDVWGQGTTVTVSS

SEQ ID NO: 263 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRNYYNYYGLDVWGQGTTVTVSS

SEQ ID NO: 264 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRSYVYYYGLDVWGQGTTVTVSS

SEQ ID NO: 265 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRSYYNYYGLDVWGQGTTVTVSS

SEQ ID NO: 266 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRNYVNYYGLDVWGQGTTVTVSS

SEQ ID NO: 267 - protein, 121 aa, synthetic construct
EVQLVESGGGVVQPGRSLRLSCAASGFTFSSYVVHWVRQAPGKGLEWVAVISYDGDNKFYADSVKGRFTISRDNSKNTLYLQMNSLRTEDTAVYYCARGRSYVNYYGLDVWGQGTTVTVSS